(19) United States
(12) Patent Application Publication
(10) Pub. No.: US 2009/0203605 A1
(43) Pub. Date: Aug. 13, 2009

(54) Methods For Treating A Condition Characterized By Dysfunction In Protein Homeostasis

(75) Inventors: Laura Segatori, Houston, TX (US); Ting-Wei Mu, San Diego, CA (US); Jeffrey W. Kelly, La Jolla, CA (US)

Correspondence Address: FENWICK & WEST LLP, SILICON VALLEY CENTER, 801 CALIFORNIA STREET, MOUNTAIN VIEW, CA, 94041, US

(73) Assignee: THE SCRIPPS RESEARCH INSTITUTE, LA JOLLA, CA (US)

(21) Appl. No.: 12/364,441

(22) Filed: Feb. 2, 2009

Related U.S. Application Data
(60) Provisional application No. 61/025,705, filed on Feb. 1, 2008.

(51) Int. Cl. A61K 38/16; A61K 31/7088; A61P 3/00

(52) U.S. Cl. 514/12

(57) ABSTRACT

Methods are provided for treating conditions characterized by dysfunction in protein homeostasis in a patient in need thereof. A method for treating a condition characterized by dysfunction in protein homeostasis in a patient in need thereof is provided which comprises administering to the patient a proteostasis regulator in an amount effective to improve or restore protein homeostasis, and to reduce or eliminate the condition in the patient or to prevent its occurrence or recurrence. The condition can be a loss of function disorder such as a lysosomal storage disease, or a gain of function disorder such as an aging associated disease.

### CROSS REFERENCE TO RELATED APPLICATIONS

This application claims the benefit of U.S. Provisional Application No. 61/025,705, filed Feb. 1, 2008, which is incorporated herein by reference in its entirety.

### STATEMENT OF GOVERNMENT SUPPORT

This invention was made by government support by Grant No. DK75295 from National Institutes of Health. The United States Government has certain rights in this invention.

### FIELD

The present invention relates generally to methods for treating conditions characterized by dysfunction in protein homeostasis in a patient in need thereof. A method for treating a condition characterized by dysfunction in protein homeostasis in a patient in need thereof is provided which comprises administering to the patient a proteostasis regulator in an amount and at dosing intervals effective to improve or restore protein homeostasis, and to reduce or eliminate the condition in the patient or to prevent its occurrence or recurrence.

### BACKGROUND

Cells normally maintain a balance between protein synthesis, folding, trafficking, aggregation, and degradation, referred to as protein homeostasis, utilizing sensors and networks of pathways. Sitia et al., Nature 426: 891-894, 2003; Ron et al., Nat Rev Mol Cell Biol 8: 519-529, 2007. Human loss of function diseases are often the result of a disruption of normal protein homeostasis, typically caused by a mutation in a given protein that compromises its cellular folding, leading to efficient degradation. Cohen et al., Nature 426: 905-909, 2003. Thus, there is insufficient function because the concentration of the mutant protein is exceedingly low.

There are at least 40 distinct lysosomal storage diseases (LSDs) resulting from the deficient function of a single mutated enzyme in the lysosome, leading to accumulation of corresponding substrate(s). Futerman et al., Nat Rev Mol Cell Biol 5: 554-565, 2004; Sawkar et al., Cell Mol Life Sci 63: 1179-1192, 2006. Currently, LSDs are treated by enzyme replacement therapy, which can be challenging because the endocytic system has to be utilized to get the recombinant enzyme into the lysosome. Desnick et al., Nat Rev Genet. 3: 954-966, 2002.

The most prevalent LSD is Gaucher disease (GD), caused by a deficiency in the activity of glucocerebrosidase (GC), a glycolipid hydrolase. Zhao et al., Cell Mol Life Sci 59: 694-707, 2002. Glucosylceramide accumulation in Gaucher monocyte-macrophage cells leads to hepatomegaly, splenomegaly, anemia, thrombocytopenia, bone lesions, and in severe cases, central nervous system (CNS) involvement. Beutler et al., The Metabolic and Molecular Bases of Inherited Diseases, New York: McGraw-Hill, 3635-3668, 2001. Patients without CNS involvement are classified as type I (mild adult onset), while those with CNS involvement are classified as type II (acute infantile onset) or type III (subacute juvenile or early adult onset). The clinically most important GC mutations, such as N370S, the most common mutation associated with type I GD, and L444P, the most prevalent mutation resulting in CNS involvement, predispose GC to misfold in the endoplasmic reticulum (ER), subjecting these variants to ER-associated degradation (ERAD), reducing the normal amount of mutant GC trafficking to the lysosome. Thus the mutant GC concentration in the lysosome is substantially reduced. Ron et al., Hum Mol Genet. 14: 2387-2398, 2005; Sawkar et al., ACS Chem Biol 1: 235-251, 2006. Many of the folding deficient GC variants exhibit fractional specific activity when properly folded, demonstrating that if folding and trafficking of the mutated enzymes could be enhanced, it is likely that the disease would be ameliorated. Liou et al., J Biol Chem 281: 4242-4253, 2006.

The FDA has approved enzyme replacement therapy and substrate reduction therapy to treat type I Gaucher disease. Sawkar et al., Cell Mol Life Sci 63: 1179-1192, 2006; Futerman et al., Trends Pharmacol Sci 25: 147-151, 2004. There is currently no effective treatment for neuropathic Gaucher disease (types II and III); the recombinant GC enzyme does not cross the blood-brain barrier and the efficacy of the substrate reduction drug in the CNS remains unclear, hence a novel strategy for neuropathic Gaucher's disease would be welcomed. Pharmacological chaperoning is an emerging therapeutic strategy that uses ER permeable small molecules that bind to and stabilize the folded state of a given enzyme, enabling its trafficking to the Golgi and onward to the lysosome. Fan et al., Nat Med 5: 112-115, 1999; Sawkar et al., Proc Natl Acad Sci USA 99: 15428-15433, 2002; Matsuda et al., Proc Natl Acad Sci USA 100: 15912-15917, 2003; Alfonso et al., Blood Cells Mol Dis 35: 268-276, 2005; Sawkar et al. Chem Biol 12: 1235-1244, 2005; Steet et al., Proc Natl Acad Sci USA 103: 13813-13818, 2006; Lieberman et al., Nat Chem Biol 3: 101-107, 2007; Parenti et al., Mol Ther 15: 508-514, 2007; Tropak et al., Chem Biol 14: 153-164, 2007; Yu et al., I J Med Chem 50: 94-100, 2007; Zheng et al., Proc Natl Acad Sci USA 104: 13192-13197, 2007. While patient derived cells harboring most GD-associated mutations appear to be amenable to pharmacological chaperoning, cell lines harboring the L444P GC mutation have thus far proven refractory, although alternative dosing regimens could ultimately be useful. Sawkar et al. Chem Biol 12: 1235-1244, 2005.

α-Mannosidosis and type IIIA mucopolysaccharidosis (MPS) are neuropathic LSDs caused by the inability of the lysosome to degrade glycoproteins and heparan sulfate, respectively. Sawkar et al., Cell Mol Life Sci 63: 1179-1192, 2006; Michalski et al, Biochim Biophys Acta-Mol Basis Dis 1455: 69-84, 1999; Yogalingam et al., Hum Mutat 18: 264-281, 2001. The P356R mutation in lysosomal α-mannosidase alters the folding energy landscape resulting in severe infantile α-mannosidosis associated with rapid mental deterioration. Gotoda et al., Am J Hum Genet. 63: 1015-1024, 1998. The prevalent S66W or R245H sulfamidase mutations in type IIIA MPS reduce mutant enzyme concentrations in the lysosome, most likely due to impaired folding and ERAD in lieu of efficient folding and trafficking of sulfamidase, leading to accumulation of heparan sulfate and severe CNS degeneration. Perkins et al., J Biol Chem 274: 37193-37199, 1999. Currently no effective therapy is available for α-mannosidosis or type IIIA MPS, hence new strategies for these neuropathic LSDs would be welcomed.

The cellular maintenance of protein homeostasis, or proteostasis, refers to controlling the conformation, binding interactions, location and concentration of individual proteins making up the proteome. Since proteins play a central role in the physiology of all organisms, loss of the normal balance between proper protein folding, localization and degradation influences or causes numerous diseases. Albanese, V., et al., Cell 124: 75-88, 2006; Brown et al., J Clin Invest 99: 1432-1444, 1997; Cohen et al., Nature 426: 905-909, 2003; Deuerling et al., Crit Rev Biochem Molec Biol 39: 261-277, 2004; Horwich et al., Encyclopedia Biol Chem 1: 393-398, 2004; Imai et al., Cell Cycle 2: 585-589, 2003; Kaufman, J Clin Invest 110: 1389-1398, 2002; Ron et al., Nat Rev Mol Cell Biol 8: 519-529, 2007; Young et al., Nat Rev Mol Cell Biol 5: 781-791, 2004. Protein folding in vivo is accomplished through interactions between the folding polypeptide chain and macromolecular cellular components, including multiple classes of chaperones and folding enzymes, which minimize aggregation. Wiseman et al., Cell 131: 809-821, 2007. Metabolic enzymes also influence cellular protein folding efficiency because the organic and inorganic solutes produced by a given compartment effect polypeptide chain salvation through non-covalent forces, including the hydrophobic effect, that influences the physical chemistry of folding. Metabolic pathways also produce small molecule ligands that can bind to and stabilize the folded state of a specific protein, enhancing folding by shifting folding equilibria. Fan et al., Nature Med., 5, 112 (January 1999); Hammarstrom et al., Science 299, 713 (2003). Whether a given protein folds in a certain cell type depends on the distribution, concentration, and subcellular localization of chaperones, folding enzymes, metabolites and the like. Wiseman et al., Cell 131: 809-821, 2007.

Loss-of-function diseases are often caused by the inability of a mutated protein to fold properly within and traffic through the secretory pathway, leading to extensive endoplasmic reticulum (ER) associated degradation (ERAD) and thus to a lowered concentration of the protein in its destination environment. Brodsky, Biochem J 404: 353-363, 2007; Brown et al., J Clin Invest 99: 1432-1444, 1997; Cohen et al., Nature 426: 905-909, 2003; Moyer et al., Emerg Ther Targets 5: 165-176, 2001; Sawkar et al, Cell Mol Life Sci 63: 1179-1192, 2006a; Schroeder et al., Ann Rev Biochem 74: 739-789, 2005; Ulloa-Aguirre et al., Traffic 5: 821-837, 2004; Wang et al., Cell 127: 803-815, 2006; Wiseman et al., Cell 131: 809-821, 2007. Lysosomal storage diseases (LSDs) are loss-of-function diseases often caused by extensive ERAD of a mutated lysosomal enzyme instead of proper folding and lysosomal trafficking. Fan, Front Biotechnol Pharm 2: 275-291, 2001; Fan et al., Nat Med 5: 112-115, 1999; Futerman et al., Nat Rev Mol Cell Biol 5: 554-565, 2004; Sawkar et al., Chem Biol 12: 1235-1244, 2005; Sawkar et al., Proc Natl Acad Sci USA 99:15428-15433, 2002; Sawkar et al, Cell Mol Life Sci 63: 1179-1192, 2006a; Sawkar et al., ACS Chem Biol 1: 235-251, 2006b; Schmitz et al., Int J Biochem Cell Biol 37: 2310-2320, 2005; Yu et al., J Med Chem 50: 94-100, 2007b; Zimmer et al., J Pathol 188: 407-414, 1999. They are characterized by substrate accumulation, which typically arises when the activity of a mutated lysosomal enzyme drops below ≈10% of normal. Conzelmann et al., Dev Neurosci 6: 58-71, 1984; Schueler et al., J Inherit Metab Dis 27: 649-658, 2004. LSDs are now treated by replacing the damaged enzyme with a wild type recombinant version that utilizes the endocytic pathway to reach the lysosome. Futerman et al., Nat Rev Mol Cell Biol 5: 554-565, 2004; Beutler et al., Proc Natl Acad Sci USA 74: 4620-4623, 1977; Brady, Ann Rev Med 57: 283-296, 2006 Enzyme replacement therapy fails for neuropathic LSDs because the recombinant enzyme does not cross the blood brain barrier. Sawkar et al, Cell Mol Life Sci 63: 1179-1192, 2006a. Many of mutated lysosomal enzymes that misfold and are degraded by ERAD can fold and exhibit partial activity under appropriate conditions, such as when the cells are grown at a lower permissive temperature. Futerman et al., Nat Rev Mol Cell Biol 5: 554-565, 2004; Sawkar et al., ACS Chem Biol 1: 235-251, 2006b. The challenge for most mutated glycolipid processing enzymes is to fold in the neutral pH environment of the ER, distinct from that of the acidic environment of the lysosome. Sawkar et al., ACS Chem Biol 1: 235-251, 2006b.

New strategies are needed to develop effective therapies for diseases related to intracellular protein misfolding and altered protein trafficking which can lead to loss of function diseases such as lysosomal storage disease and neuropathic lysosomal storage disease, or gain of function disease such as age-onset related disease, e.g., age-related macular degeneration, inclusion body myositosis, type II diabetes, amyotrophic lateral sclerosis, Alzheimer's disease, Huntington's disease or Parkinson's disease. Since current treatments are limited to compounds approved for enzyme replacement therapy or substrate reduction therapy, a need exists in the art for new therapeutic approaches to treat protein loss of function diseases or gain of function diseases related to dysfunction in protein homeostasis.

### SUMMARY

The present invention relates generally to methods for treating conditions characterized by dysfunction in protein homeostasis in a patient in need thereof. The dysfunction in protein homeostasis can be a result of protein misfolding, protein aggregation, defective protein trafficking, protein degradation or combinations thereof. The method can comprise administering to the patient a proteostasis regulator in an amount and dosing schedule effective to improve or restore protein homeostasis. The proteostasis regulator can act via a cellular mechanism that upregulates signaling via a heat shock response (HSR) pathway and/or an unfolded protein response (UPR) pathway or through aging-associated signaling pathways that besides controlling longevity and youthfulness control protein homeostasis capacity.

A method for treating a condition characterized by dysfunction in protein homeostasis in a patient in need thereof is provided which comprises administering to the patient a proteostasis regulator in an amount effective to improve or restore protein homeostasis, and to reduce or eliminate the condition in the patient or to prevent its occurrence or recurrence. The condition can be a loss of function disorder, e.g., a lysosomal storage disease, α1-antitrypsin-associated emphysema, or cystic fibrosis. The condition includes, but is not limited to, Gaucher's disease, α-mannosidosis, type IIIA mucopolysaccharidosis, Fabry disease, Tay-Sach's disease, and Pompe disease. The proteostasis regulator can upregulate coordinately transcription or translation of a chaperone network or a fraction of a network or impede turnover of network components or the proteostasis regulator can inhibit the degradation of a mutant protein. The condition can be a gain of function disorder, for example, a disorder causing disease such as inclusion body myositis, amyotrophic lateral sclerosis, age-related macular degeneration, Alzheimer's disease, Huntington's disease or Parkinson's disease. Treatment of a disease or condition with the proteostasis regulator can upregulate signaling via a heat shock response (HSR) pathway and/or an unfolded protein response (UPR) pathway, including upregulation of genes or gene products associated with these pathways. The proteostasis regulator can regulate protein chaperones and/or folding enzymes by upregulating transcription or translation of the protein chaperone, or inhibiting degradation of the protein chaperone. The proteostasis regulator can upregulate an aggregation pathway or a disaggregase activity. The proteostasis regulator can inhibit degradation of one or more protein chaperones, one or more folding enzymes, or a combination thereof. Altering signaling pathways associated with aging is another approach for regulating protein homeostasis pathways. Altering intracellular Ca⁺⁺ ion concentrations is a further approach to coordinatively enhanced protein homeostasis capacity.

The proteostasis regulator can be a composition which includes, but is not limited to, a small chemical molecule, a protein, an antisense nucleic acid, short hairpin RNA, short interfering RNA or ribozyme. The proteostasis regulator can be administered in an amount that does not increase susceptibility of the patient to viral infection or susceptibility to cancer.

In a further aspect, the method for treatment can further comprise administering a pharmacologic chaperone or kinetic stabilizer. The method for treatment can further comprise administering a second mechanistically distinct proteostasis regulator. The first and the second proteostasis regulator can be one or more of aggregation regulator, disaggregation regulator, protein degradation regulator or protein folding regulator.

A method for treating a condition characterized by dysfunction in protein homeostasis in a patient in need thereof is provided which comprises administering to said patient a proteostasis regulator in combination with a pharmacologic chaperone or kinetic stabilizer in an amount effective to improve or restore protein homeostasis and to reduce or eliminate the condition in the patient or to prevent its occurrence or recurrence. The condition can be a loss of function disorder. The proteostasis regulator promotes correct folding of a mutated enzyme, for example, a lysosomal enzyme. The method for treatment can further comprise administering a polynucleotide or polypeptide encoding a lysosomal enzyme having normal activity to replace the mutated lysosomal enzyme. In a further aspect, the proteostasis regulator can inhibit endoplasmic reticulum associated degradation. The condition can be Gaucher's disease. The pharmacologic chaperone can be N-(n-nonyl)deoxynojirimycin. The condition can be Tay-Sach's disease, and the pharmacologic chaperone can be 2-acetamido-2-deoxynojirimycin. In a further aspect, the condition can be a gain of function disorder. The condition includes, but is not limited to, inclusion body myositis, age-related macular degeneration, amyotrophic lateral sclerosis, Alzheimer's disease, Huntington's disease or Parkinson's disease.

A method for treating a loss of function disease in a patient in need thereof is provided which comprises administering to said patient a proteostasis regulator in an amount effective to improve or restore activity of a mutated protein and to reduce or eliminate the loss of function disease in the patient or to prevent its occurrence or recurrence. The method for treatment can further comprise administering a polynucleotide or polypeptide encoding a protein having normal activity to replace the mutated protein.

In one aspect, said proteostasis regulator promotes correct folding of the mutated protein, and wherein said proteostasis regulator does not bind to the mutated protein. The proteostasis regulator can reduce or eliminate endoplasmic reticulum associated degradation of a protein chaperone. The proteostasis regulator can be a proteasome inhibitor. In one aspect, the loss of function disease can be cystic fibrosis and the mutated protein can be cystic fibrosis transmembrane conductance regulator (CFTR).

In a further aspect, the proteostasis regulator increases the concentration of Ca2+ in the endoplasmic reticulum and/or decreases the concentration of Ca2+ in the cytosol. In yet another aspect, the proteostasis regulator is a Ca²⁺ channel blocker. In another embodiment, the proteostasis regulator is an agent that inhibits a ryanodine receptor (RyR).

In yet another embodiment, the proteostasis regulator is diltiazem or verapamil.

The loss of function disease can be a lysosomal storage disease and the mutated protein can be a lysosomal enzyme. The lysosomal storage disease can be a neuropathic lysosomal storage disease, Gaucher's disease, neuropathic Gaucher's disease. α-mannosidosis, type IIIA mucopolysaccharidosis, Fabry disease, Tay-Sach's disease or Pompe disease. The lysosomal storage disease can be Gaucher's disease, and the enzyme can be glucocerebrosidase, or for example, a mutant enzyme L444P glucocerebrosidase or N370S glucocerebrosidase. lysosomal storage disease can be α-mannosidosis, and the enzyme can beα-mannosidase or for example, a mutant enzyme P356R α-mannosidase. The lysosomal storage disease can be type IIIA mucopolysaccharidosis, and the enzyme can be sulfamidase, for example, S66W sulfamidase or R245H sulfamidase. In a further aspect, the disease is Tay-Sach's disease, and the enzyme is β-hexosamine A, or the mutant enzyme, G269S β-hexosamine A. The proteostasis regulator can be, for example, celastrol or MG-132.

A method for treating a condition characterized by a dysfunction in protein homeostasis in a patient in need thereof is provided which comprises administering to said patient at least two mechanistically distinct proteostasis regulators wherein said proteostasis regulators are administered in an amount effective to improve or restore protein homeostasis and to reduce or eliminate the condition in the patient or to prevent its occurrence or recurrence. At least one of said proteostasis regulators can enhance correct folding of a mutated protein. At least one of said proteostasis regulators can inhibit endoplasmic reticulum associated degradation of a mutated protein. In a further aspect, the mutated protein can be a mutated enzyme.

A method for diagnosing a condition characterized by a dysfunction in protein homeostasis in a patient is provided which comprises contacting cells or tissue from the patient with a proteostasis regulator in a cell-based assay system, measuring an effect of the proteostasis regulator on protein folding, protein aggregation, protein trafficking or protein degradation in the cell, and identifying a deficiency in the protein homeostasis in the cells or tissue of the patient. The condition can be a loss of function disorder and the method can further comprise identifying a deficiency in the folding or trafficking of the protein. The condition can be a gain of function disorder and the method can further comprise identifying a deficiency in the degradation of the protein. The deficiency can be in the synthesis of a protein chaperone. The proteostasis regulator can upregulate signaling via a heat shock response (HSR) pathway or an unfolded protein response (UPR) pathway, or a combination thereof. The proteostasis regulator can upregulate transcription or translation of one or more protein chaperones, one or more folding enzymes, or a combination thereof. The proteostasis regulator can inhibit degradation of one or more protein chaperones, one or more folding enzymes, or a combination thereof. The proteostasis regulator can upregulate an aggregation pathway or a disaggregation pathway.

A method for designing a treatment regimen by identifying two or more proteostasis components is provided which comprises comparing the activities of the proteostasis components with a standard; selecting proteostasis regulators to modify the activities of the proteostasis components towards the activities of the standard; and administering said regulators to a patient in need thereof.

### BRIEF DESCRIPTION OF THE DRAWINGS

FIGS. 1A, 1B, and 1C show Celastrol treatment enhances activity of variant glucocerebrosidases (GCs) and their cellular trafficking to the lysosome.

FIGS. 2A, 2B and 2C show the proteasome inhibitor MG-132 potently enhances GC activity and promotes cellular trafficking of GC to the lysosome within L444P GC fibroblasts.

FIGS. 3A, 3B, 3C, 3D, 3E, and 3F show pharmacologic chaperones and proteostasis regulators exhibit synergy in enhancing folding, trafficking, and cellular enzyme activity.

FIGS. 4A, 4B, 4C, and 4D show PR alone, or in combination with an enzyme-specific pharmacologic chaperone, enhances Hex α-site activity of a G269S/1278insTATC HexA Tay-Sachs fibroblast cell line.

FIGS. 5A, 5B, 5C, and 5D show both MG-132 and celastrol activate the heat shock response in L444P GC fibroblasts.

FIGS. 6A, 6B, 6C, 6D, and 6E show GC proteostasis regulation by MG-132 and celastrol can occur through the unfolded folded protein response.

FIG. 7 shows GC proteostasis restoration pathways and integrates the data from FIGS. 5 and 6 demonstrating that in some cases PR upregulate components of both the HSR and the UPR. As shown schematically in FIG. 30, PR can also regulate one or more aspects of Ca²⁺ homesostasis.

FIG. 8 shows Western blot analysis of GC trafficking in L444P GC fibroblasts.

FIGS. 9A, 9B, and 9C show optimization of celastrol dosing regime in L444P GC fibroblasts.

FIG. 10 shows the effect of proteasome inhibitors on GC activity in L444P GC fibroblasts.

FIG. 11 shows the effect of MG-132 and celastrol on the activity of other WT lysosomal enzymes in L444P fibroblasts, as well as GC in WT GC fibroblasts.

FIGS. 12A, 12B, 12C, and 12D show two dimensional plots showing GC activity of G202R and N370S GC patient derived fibroblasts cultured with media containing celastrol and NN-DNJ.

FIGS. 13A and 13B show cells were plated and treated according to the same experimental design described in FIG. 12 with the exception that the incubation medium was replaced at t=0, 30, 60, 72, 102, and 132 h.

FIGS. 14A, 14B, 14C, and 14D show relative L444P GC activity in patient derived fibroblasts cultured with media containing MG-132 and celastrol, or MG-132 and NN-DNJ.

FIGS. 15A and 15B show relative Hex α-site activity in G269S/1278insTATC HexA Tay-Sachs fibroblast cell line cultured with media containing MG-132 and ADNJ.

FIG. 16 shows the effect of Compound 101, an Hsp70 inhibitor alone, or in combination with MG-132 on GC activity in L444P GC fibroblasts.

FIGS. 17A, 17B, 17C, 17D, 17E, and 17F show influence of small molecules on glucocerebrosidase (GC) variant activity in Gaucher patient-derived fibroblasts.

FIGS. 18A, 18B, 18C, 18D, 18E, 18F, 18G, and 18H show effect of diltiazem on L444P and N370S/V394L GC folding and trafficking.

FIG. 19 shows intracellular Ca²⁺ ion concentration influences GC activity in L444P and N370S/V394L GC fibroblasts.

FIG. 20 shows chaperone expression level in untreated and diltiazem-treated L444P GC fibroblasts.

FIG. 21 shows the influence of diltiazem and verapamil on mutant α-mannosidase and heparan sulfate sulfamidase (SGSH) activity in patient-derived fibroblasts.

FIG. 22 shows the influence of ruthenium red on L444P glucocerebrosidase (GC) activity in Gaucher patient-derived fibroblasts after culturing for one to five days.

FIG. 23 shows the influence of diltiazem on the activity of lysosomal enzymes.

FIG. 24 shows GC activities of L444P and N370S/V394L GC cells incubated with diltiazem for 1 hour, as determined using the intact cell GC activity assay.

FIG. 25 shows the influence of thapsigargin and diltiazem on GC activity in L444P GC fibroblasts.

FIG. 26 shows quantitative RT-PCR analysis on untreated and diltiazem-treated N370S/V394L GC cells.

FIG. 27 shows siRNA knockdown of IRE1α or PERK blocks the ability of MG-132 (0.25 μM in DMSO) to increase L444P GC activity, activities normalized to L444P GC cells treated with both nontargeting siRNA (control), and DMSO vehicle.

FIGS. 28A and 28B show Western blot analyses of L444P GC in fibroblasts treated with nontargeting siRNA (control) plus DMSO (vehicle) or HSF1, IRE1α, ATF6, and PERK siRNAs without (just DMSO vehicle) or with 0.25 μM MG-132 (A) or 0.8 μM celastrol (B) in DMSO.

FIG. 29 shows changes in the L444P GC fibroblast proteome (A) after MG-132 (0.8 μM) or celastrol (0.8 μM) treatment for 72 hr. The number of proteins is plotted against fold change on a log₂ (upper) and log₁₀ (lower) scales using a normalized spectra count ratio of drug-treated samples versus untreated samples in cases where a given protein is detected in both untreated and treated samples.

FIG. 30 shows a schematic illustration of Ca²⁺ homeostasis in the endoplasmic reticulum (ER). Ca²⁺ levels are controlled by a number of systems, including the IP₃ receptor (IP₃R) and ryanodine receptor (RyR) release channels, and the sarco/endoplasmic reticulum Ca²⁺-ATPase (SERCA) pump.

FIGS. 31A and 31B show relative glucocerebrosidase (GC) activity in L444P (A) and N370S (B) fibroblasts in the presence of the RyR inhibitor dantrolene.

FIG. 32 shows a Western blot analysis of Endo H sensitivity of L444P fibroblasts before and after exposure to the RyR inhibitor dantrolene.

FIGS. 33A, 33B, 33C, 33D and 33E show relative GC activity in L444P fibroblasts upon treatment with the IP₃R inhibitors XeC (A), chloroquinine (B), quinine (C), thimerosal (D) and KN93 (E).

FIG. 34 shows relative mRNA expression levels of GC and large ribosomal protein (RiboP) control in L444P fibroblasts after treatment with the RyR inhibitor dantrolene.

FIGS. 35A and 35B show the Endo H sensitivity (A) and relative GC activity of L444P fibroblasts overexpressing the SERCA2 pump (A).

FIGS. 36A and 36B show cytoplasmic Ca²⁺ levels in L444P GC fibroblasts after varying exposures to diltiazem.

FIGS. 37A, 37B and 37C show the Endo H sensitivity (A, B) and relative GC activity (C) of L444P fibroblasts after exposure to siRNA against RyR1, RyR2 and RyR3 and the combinations RyR1/3 and RyR2/3.

FIG. 38 shows relative expression levels of RyR1, RyR2 and RyR3 in L444P GC fibroblasts, indicating that RyR3 is the most abundantly expressed isoform.

FIGS. 39A, 39B and 39C show levels of binding between L444P GC protein and the ER chaperone calnexin after exposure to dantrolene (A), dantrolene plus EDTA (B), or diltiazem (C).

FIGS. 40A and 40B show levels of binding between L444P, N370S and G202R GC proteins and the ER chaperones calnexin, calreticulin, and BiP (A) and the binding between wt GC protein and calreticulin (CRT) after exposure to dantrolene (B).

FIG. 41 shows relative expression levels of the cytoplasmic chaperones Hsp40, Hsp70, Hsp90, Hsp27, and αβ-crystallin (CRYAB) in L444P GC fibroblasts after varying exposures to dantrolene.

FIGS. 42A and 42B show relative expression levels of the ER-associated proteins C/EBP homologous protein (CHOP) and X box binding protein 1 (XBP-1) (A), and the ER-associated chaperones BiP, CRT and GRP94, the ER-associated folding enzymes ERp57 and protein disulphide isomerase (PDI), and the cytoplasmic chaperones Hsp70 and Hsp90 (B) after exposure to dantrolene.

FIGS. 43A and 43B show the Endo H sensitivity (A) and relative GC activity (B) of L444P fibroblasts overexpressing calnexin.

FIG. 44 shows relative GC activity of N370S fibroblasts in the presence of dantrolene, both alone and in combination with a pharmacologic chaperone.

### DETAILED DESCRIPTION

The present invention relates to methods for treating conditions characterized by dysfunction in protein homeostasis resulting in gain-of-function and loss-of-function diseases in patients in need thereof. The conditions encompass metabolic, oncologic, neurodegenerative and cardiovascular disorders. Loss-of-function diseases, e.g., lysosomal storage diseases (LSDs) including the neuropathic variety, cystic fibrosis, or α1-antitrypsin deficiency-associated emphysema, are often caused by dysfunction in protein homeostasis, or proteostasis, sometimes resulting from mutations in proteins traversing the secretory pathway that compromise the normal balance between protein folding, trafficking and degradation. Gain of function disease often are age-onset related disease, e.g., amyotrophic lateral sclerosis, age-related macular degeneration, inclusion body myositosis, Alzheimer's disease, Huntington's disease or Parkinson's disease. As described herein, the innate cellular protein homeostasis machinery can be adapted to fold mutated enzymes that would otherwise misfold and be degraded, by administering to the cell proteostasis regulators e.g., small chemical compound proteostasis regulators, RNAi, shRNA, ribozymes, antisense RNA, or proteins, protein analogs or mimetics. The present invention provides methods for treating conditions characterized by dysfunction in protein homeostasis by administering proteostasis regulators which, by altering the composition of the proteostasis environment of the cytoplasm and/or the endoplasmic reticulum, can partially restore folding, trafficking and function to non-homologous mutant enzymes, each associated with a distinct lysosomal storage disease. A further synergistic restoration of proteostasis was observed when an enzyme-specific pharmacologic chaperone was co-administered with a proteostasis regulator, owing to their distinct mechanisms of action. It may be possible to ameliorate loss-of-function and/or gain-of-function diseases by administering proteostasis regulators or administering a combination of a pharmacologic chaperone and a proteostasis regulator.

A method for treating a condition characterized by dysfunction in protein homeostasis in a patient in need thereof is provided which comprises administering to the patient a proteostasis regulator in an amount and dosing schedule effective to improve or restore protein homeostasis, and to reduce or eliminate the condition in the patient or to prevent its occurrence or recurrence. The condition can be a loss of function disorder, e.g., a lysosomal storage disease. The condition includes, but is not limited to, Gaucher's disease, α-mannosidosis, type IIIA mucopolysaccharidosis, Fabry disease, Tay-Sach's disease, Pompe disease, cystic fibrosis, and α1-antitrypsin deficiency-associated emphysema. The proteostasis regulator can upregulate transcription or translation of a protein chaperone or chaperone network, or inhibit the degradation of a protein chaperone or chaperone network. The condition can be a gain of function disorder, for example, a disorder causing disease such as inclusion body myositis, amyotrophic lateral sclerosis, age-related macular degeneration, Alzheimer's disease, Huntington's disease or Parkinson's disease. Treatment of a disease or condition with the proteostasis regulator can coordinately upregulate signaling via a heat shock response (HSR) pathway and/or an unfolded protein response (UPR) pathway, including upregulation of genes or gene products associated with these pathways. It is also clear that affecting signaling pathways associated with longevity and youthfulness is another approach to regulate the proteostasis network.

Methods for treating loss-of-function conditions characterized by dysfunction in protein homeostasis in a patient in need thereof support a therapeutic strategy wherein instead of replacing damaged enzymes, it would be possible to restore partial folding, trafficking and function to misfolding and degradation prone (ER-associated degradation, ERAD) mutated lysosomal enzymes by adapting the innate cellular biology of proteostasis. Similarly, adapting the cellular biology or proteostasis can be used in the treatment of gain of function diseases in place of or in addition to kinetic stabilizers, small molecules that bind to the folded functional state of a protein to impose kinetic stability on it and thereby prevent denaturation and misassembly into aggregates. Small chemical molecules or biologicals (protein mimetics or analogs, RNAi, shRNA, ribozymes, or antisense RNA) that enhance cellular protein homeostasis, or “proteostasis regulators”, often function by manipulating signaling pathways, including the heat shock response, the unfolded protein response, and longevity-associated signaling pathways, resulting in transcription and translation of proteostasis network components. For example, the small chemical compound, celastrol, activates the heat shock response, leading to enhanced expression of chaperones, co-chaperones, folding enzymes, and the like. Westerheide et al., J Biol Chem 279: 56053-56060, 2004; Yang et al., Cancer Res 66: 4758-4765, 2006.

A single proteostasis regulator should be able to restore proteostasis in multiple diseases, because the proteostasis network has evolved to support the folding and trafficking of many client proteins simultaneously. In addition, proteostasis regulators should complement the established utility of pharmacologic chaperones/kinetic stabilizers because of their distinct mechanisms of action. Asano et al., Eur J Biochem 267: 4179-4186, 2000; Bouvier, Chem Biol 14: 241-242, 2007; Fan et al., Nat Med 5: 112-115, 1999; Sawkar et al., Proc Natl Acad Sci USA 99:15428-15433, 2002; Brown et al., J Clin Invest 99: 1432-1444, 1997; Ulloa-Aguirre et al., Traffic 5: 821-837, 2004. Pharmacologic chaperones/kinetic stabilizers bind an existing steady state level of the folded mutant protein and chemically enhance the folding equilibrium by stabilizing the fold. Bouvier, Chem Biol 14: 241-242, 2007; Fan et al., Nat Med 5: 112-115, 1999; Sawkar et al., Proc Natl Acad Sci USA 99:15428-15433, 2002. In contrast, proteostasis regulators influence the biology of folding, often by the coordinated increase in chaperone and folding enzyme levels and macromolecules that bind to partially folded conformational ensembles, thus enabling their progression to intermediates with more native structure and ultimately increasing the concentration of folded mutant protein for export.

The methods for treating conditions characterized by a dysfunction in protein homeostasis focus on discovering proteostasis regulators that function in patient-derived cell lines from dissimilar lysosomal storage diseases (LSDs). The most common LSD, Gaucher disease, is typically caused by N370S or L444P glucocerebrosidase (GC) mutations that lead to extensive ERAD and loss of GC function in the lysosome, resulting in glucosylceramide accumulation. Beutler et al., Blood Cells Mol Dis 35: 355-364, 2005; Sawkar et al, Cell Mol Life Sci 63: 1179-1192, 2006a; Sawkar et al., ACS Chem Biol 1: 235-251, 2006b. The L444P mutation, which often leads to neuropathic Gaucher disease, does not respond significantly to pharmacologic chaperones (unlike the N370S variant) presumably because of the very low concentration of folded L444P GC. Sawkar et al., Chem Biol 12: 1235-1244, 2005; Sawkar et al., Proc Natl Acad Sci USA 99:15428-15433, 2002; Sawkar et al., ACS Chem Biol 1: 235-251, 2006b; Yu et al., J Med Chem 50: 94-100, 2007b. Tay-Sachs disease (TSD) is another loss-of-function LSD that can be caused by β-hexosaminidase A (HexA) mutations including G269S. Jeyakumar et al., Neuropathol Appl Neurobiol 28: 343-357, 2002. This mutation in the α-subunit compromises the folding and trafficking of HexA, a heterodimeric glycoprotein composed of α- and β-subunits, leading to substantial ERAD, and neuronal storage of GM2 gangliosides, its substrate. Maegawa et al., J Biol Chem 282: 9150-9161, 2007. The folding, trafficking and activity of HexA is known to be partially restored in patient-derived fibroblasts harboring the G269S α-subunit mutation upon active site directed pharmacologic chaperone treatment. Maegawa et al., J Biol Chem 282: 9150-9161, 2007; Tropak et al., J Biol Chem 279: 13478-13487, 2004.

Two proteostasis regulators are described herein that each partially restore glucocerebrosidase and HexA proteostasis and function in Gaucher and Tay-Sachs patient-derived cell lines, providing proof of principle that it is possible to treat multiple LSDs with a single proteostasis regulator. These proteostasis regulators appear to function by activating both the heat shock response and the unfolded protein response, altering the proteostasis components within the cytoplasm and the ER, respectively. Moreover, in each case these results demonstrate that the combination of a proteostasis regulator with an active site directed pharmacologic chaperone yields synergistic restoration of the mutant enzyme function in patient-derived fibroblasts, as a consequence of their distinct mechanisms of action.

Whether the activation of both the heat shock response and the unfolded protein response is required for a specific application is discerned by applying a proteostasis regulator and an RNAi or siRNA to HSF1 that initiates the heat shock response signaling pathway or a proteostasis regulator and a RNAi or siRNA to components required to activate the three arms of the unfolded protein response signaling pathway. The requirements of individual components (chaperones, folding enzymes, metabolites) can also be discerned by applying a proteostasis regulator and an RNAi or siRNA to patient-derived cells. The loss of function of the proteostasis regulator upon the coadministration of a given RNAi informs one that that pathway or pathway component is critical for restoration of proteostasis.

The Ca²⁺ ion is a universal and extremely important signaling ion in the cell. Ca²⁺ signaling affects numerous cellular functions by diverse pathways, and is a primary regulator of endoplasmic reticulum (ER) function. Berridge et al., Nat Rev Mol Cell Biol 4: 517-529, 2003; Burdakov et al., Cell Calcium 38: 303-310, 2005; Gorlach et al., Antioxid Redox Signal 8: 1391-1418, 2006. Emerging evidence indicates that calcium signaling may influence diseases associated with deficiencies in protein homeostasis, including many lysosomal storage diseases (LSDs). Futerman et al., Nat Rev Mol Cell Biol 5: 554-565, 2004; LaFerla, Nat Rev Neurosci 3: 862-872, 2002; Petersen et al., Cell Calcium 38: 161-169, 2005. This hypothesis is supported by observations that manipulation of calcium homeostasis by sarcoplasmic/endoplasmic calcium (SERCA) pump inhibitors, such as thapsigargin enhances folding and trafficking of the ΔF508 cystic fibrosis transmembrane conductance regulator (CFTR) and curcumin. Egan et al., Nat Med 8: 485-492, 2002; Egan et al., Science 304: 600-602, 2004.

The invention is additionally directed to methods for treating conditions characterized by dysfunction in protein homeostasis by manipulating intracellular calcium homeostasis to improve defects in mutant enzyme homeostasis that lead to LSDs. It has been found that agents that reduce cytosolic calcium concentration and/or increase endoplasmic reticulum (ER) calcium concentration enhance the folding and activities of mutant enzymes associated with LSDs, such as Gaucher's disease, mannosidosis and mucopolysaccharidosis Type IIIA. Furthermore, as shown in the Examples below, increasing the calcium concentration in the ER enhanced the activity of calcium-binding chaperone proteins. Therefore, one embodiment of the invention is directed to the treatment of an LSD by enhancing the folding of a mutant lysosomal enzyme by administering an agent that increases the calcium concentration in the ER and/or decreases the calcium concentration in the cytosol and/or enhances the activity of calcium binding chaperones in the ER. Agents that enhance the folding, trafficking and function of endogenous mutant lysosomal enzymes in multiple cell lines associated with different LSDs, thus restoring function by repairing instead of replacing the damaged enzyme through altering calcium homeostasis were investigated and are described in detail below. For example, the FDA approved drugs diltiazem and verapamil, both L-type voltage-gated calcium channel blockers were discovered to partially restore mutant lysosomal enzyme function in three distinct LSDs caused by folding defects in nonhomologous enzymes. These results suggest that calcium channel blockers are promising candidates to enhance lysosomal enzyme homeostasis in a variety of LSDs.

LSDs result from deficient lysosomal enzyme activity, thus the substrate of the mutant enzyme accumulates in the lysosome, leading to pathology. In many but not all LSDs, the clinically most important mutations compromise the cellular folding of the enzyme, subjecting it to endoplasmic reticulum-associated degradation instead of proper folding and lysosomal trafficking. An agent, such as a small molecule or macromolecule, that restores partial mutant enzyme folding, trafficking and activity would be highly desirable, particularly if a single agent could ameliorate multiple distinct lysosomal storage diseases by virtue of its mechanism of action. Inhibition of L-type Ca²⁺ channels, using either diltiazem or verapamil, both FDA-approved hypertension drugs, partially restores N370S and L444P glucocerebrosidase (GC) homeostasis in Gaucher patient-derived fibroblasts—the latter mutation is associated with refractory neuropathic disease. Diltiazem structure-activity studies suggest that it is its Ca²⁺ channel blocker activity that enhances the capacity of the endoplasmic reticulum to fold misfolding prone proteins. Importantly, diltiazem and verapamil also partially restore enzyme homeostasis in two other distinct LSDs involving enzymes essential for glycoprotein and heparan sulfate degradation, namely α-mannosidosis and type IIIA mucopolysaccharidosis, respectively.

One embodiment of the invention is therefore directed to a method of treating an LSD comprising administering a calcium channel blocker. The term “calcium channel blocker” refers to an agent that blocks voltage-dependent calcium channels. Synonyms of the term “calcium channel blocker” are calcium channel antagonists, calcium channel inhibitors and calcium entry blockers and these terms are used interchangeably herein. Calcium channel blockers include “rate limiting” agents such as verapamil and dilitiazem and the dihydropyridine group of calcium channel blockers (Meredith et al. (2004). J of Hypertension 22: 1641-1648). Specific examples of calcium channel blockers are amlodipine, felodipine, isradipine, lacidipine, nicardipine, nifedipine, niguldipine, niludipine, nimodipine, nisoldipine, nitrendipine, nivaldipine, ryosidine, anipamil, diltiazem, fendiline, flunarizine, gallopamil, mibefradil, prenylamine, tiapamil, verapamil, perhexyline maleate, fendiline and prenylamine and salts, esters, amides, prodrugs, or other derivatives of any of thereof. In one embodiment of the invention, the calcium channel blocker is an L-type Ca2+ channel blocker. In another embodiment, the invention is a method of treating an LSD comprising inhibiting the activity of an L-type calcium channel. In a further embodiment, the invention is a method of treating an LSD comprising increasing the expression of one or more calcium-binding chaperone(s) in the ER. In an addition embodiment, the invention is a method of treating an LSD comprising increasing the activity of one or more calcium-binding chaperone(s) in the ER. In yet another embodiment, the invention is a method of increasing the expression and/or activity of one or more calcium-binding chaperone(s) in the ER by administering an L-type Ca2+ calcium channel blocker. Exemplary calcium binding chaperone proteins are BiP, calnexin and calreticulin.

Another approach to manipulating calcium homeostasis is by modulating the activity of ER calcium receptors. ER calcium receptors include, for example, ryanodine receptors (RyR), inositol 3-phosphate receptors (IP3R) and SERCA pump proteins. RyR and IP3R mediate efflux of calcium from the ER whereas SERCA pump proteins mediate influx of calcium into the ER. In one embodiment, the calcium concentration in the ER is increased by inhibiting an RyR. There are three RyR subtypes, RyR1, RyR2 and RyR3. Exemplary methods of inhibiting a RyR receptor are administration of a receptor antagonist and inhibiting the expression of the receptor, for example, by administering an antisense nucleic acid, or by using RNA or DNA interference. Exemplary RyR receptor antagonists are dantrolene, ryanodine, azumolene, calquestrin and procaine. In one embodiment, the RyR antagonist is dantrolene. In a further embodiment, the calcium concentration in the ER is increased by inhibiting at least two RyR subtypes.

In yet another embodiment, the invention is a method of treating an LSD comprising inhibiting an RyR and administering a pharmacologic chaperone. As is shown below, administration of dantrolene in combination with a pharmacologic chaperone resulted in synergism in the restoration of mutant glucocerebrosidase (GC) activity.

In a further embodiment, the invention is a method of treating an LSD comprising administering a proteostasis regulator to a patient in need thereof, wherein the proteostasis regulator is selected from the group consisting of diltiazem and verapamil and salts, esters, amides, prodrugs thereof.

It is to be understood that this invention is not limited to particular methods, reagents, compounds, compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes a combination of two or more cells, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Protein homeostasis” or “proteostasis” refers to controlling the concentration, conformation, binding interactions, e.g., quaternary structure, and location of individual proteins making up the proteome, by readapting the innate biology of the cell, often through transcriptional and translational changes. Proteostasis is influenced by the chemistry of protein folding/misfolding and by numerous regulated networks of interacting and competing biological pathways that influence protein synthesis, folding, conformation, binding interactions, trafficking, disaggregation and degradation.

In contrast to the protein replacement and pharmacologic chaperone/kinetic stabilizer approaches, methods provided herein for treatment of disease characterized by a dysfunction in protein homeostasis provide a therapeutic strategy to restore proteostasis which includes the use of proteostasis regulators. Proteostasis regulators are distinct from protein replacement and pharmacologic chaperone/kinetic stabilizer approaches. These proteostasis regulators can be small molecules or biologicals (siRNA, shRNA, antisense RNA, ribozymes, cDNA or protein) which can be used to manipulate the concentration, conformation, binding interactions, e.g., quaternary structure, and/or the location of a given protein or family of proteins by readapting the innate biology of the cell. This can be accomplished by altering the proteostasis network, including processes involved in influencing protein synthesis, folding, trafficking and degradation pathways. Proteostasis regulators often function by manipulating signaling pathways, including the heat shock response (HSR) pathway, the unfolded protein response (UPR) pathway, and Ca²⁺ signaling pathways that control longevity and protein homeostasis, and/or the transcription and translation of components of a given pathway(s) comprising the proteostasis network, including chaperones, folding enzymes, and small molecules made by metabolic pathways. Methods for treating a condition characterized by dysfunction in protein homeostasis in a patient in need thereof include both loss of function disease and gain of function disease associated with defective proteostasis, which can be remedied utilizing proteostasis regulators.

“Proteostasis regulators” refers to small molecules, siRNA, biologicals that enhance cellular protein homeostasis. Proteostasis regulators function by manipulating signaling pathways, including, but not limited to, the heat shock response or the unfolded protein response, or both, resulting in transcription and translation of proteostasis network components. For example, celastrol activates the heat shock response, leading to enhanced expression of chaperones, co-chaperones and the like. Westerheide et al., J Biol Chem 279: 56053-56060, 2004; Yang et al., Cancer Res 66: 4758-4765, 2006. Proteostasis regulators can also regulate protein chaperones by upregulating transcription or translation of the protein chaperone, or inhibiting degradation of the protein chaperone. In addition, proteostasis regulators can upregulate an aggregation pathway or a disaggregase activity. A single proteostasis regulator should be able to restore proteostasis in multiple diseases, because the proteostasis network has evolved to support the folding and trafficking of many client proteins simultaneously.

In addition, proteostasis regulators have a distinct mechanism of action from pharmacologic chaperones/kinetic stabilizers and complement the established utility of pharmacologic chaperones/kinetic stabilizers. Asano et al., Eur J Biochem 267: 4179-4186, 2000; Bouvier, Chem Biol 14: 241-242, 2007; Fan et al., Nat Med 5: 112-115, 1999; Sawkar et al., Proc Natl Acad Sci USA 99:15428-15433, 2002; Brown et al., J Clin Invest 99: 1432-1444, 1997; Ulloa-Aguirre et al., Traffic 5: 821-837, 2004. In one aspect, the proteostasis regulator is distinct from a chaperone in that the proteostasis regulator can enhance the homeostasis of a mutated protein but does not bind the mutated protein. In another aspect, a single molecule comprises a proteostasis regulator moiety and a chaperone moiety and has dual functionality.

Intracellular regulatory signaling pathways that alter proteostasis include the “heat shock response (HSR)” which regulates cytoplasmic proteostasis, the “unfolded protein response (UPR)” which maintains exocytic pathway proteostasis and pathways associated with organismal longevity control that also control protein homeostasis. These include the insulin/insulin growth factor receptor signaling pathway and pathways associated with dietary restriction as well as processes associated with the mitochondrial electron transport chain process. Temporal cellular proteostasis adaptation is necessary, due to the presence of an ever-changing proteome during development and the presence of new proteins and the accumulation of misfolded proteins upon aging. Because the fidelity of the proteome is challenged during development and aging, and by exposure to pathogens that demand high protein folding and trafficking capacity, cells utilize stress sensors and inducible pathways to respond to a loss of proteostatic control. These include the “heat shock response (HSR)” that regulates cytoplasmic proteostasis, and the “unfolded protein response (UPR)” that helps maintain exocytic pathway proteostasis.

“Pharmacologic chaperones” or “kinetic stabilizers” refer to compounds that bind an existing steady state level of the folded mutant protein and chemically enhance the folding equilibrium by stabilizing the fold. Bouvier, Chem Biol 14: 241-242, 2007; Fan et al., Nat Med 5: 112-115, 1999; Sawkar et al., Proc Natl Acad Sci USA 99:15428-15433, 2002; Johnson and Kelly, Accounts of Chemical Research 38: 911-921, 2005. In contrast, proteostasis regulators influence the biology of folding, often by a coordinated increase of chaperone/cochaperone and folding enzyme levels that bind to partially folded conformational ensembles, thus enabling their progression to intermediates with more native structure and ultimately increasing the concentration of folded mutant protein for export.

“Aggregation pathway” or “aggregation activity” refers to an activity exhibited by an organism that assembles or aggregates a protein sometimes aggregating toxic precursors into less toxic aggregates. The integrity of protein folding could play a role in lifespan determination and the amelioration of aggregation-associated proteotoxicity

“Disaggregation pathway”, “disaggregation activity”, or “disaggregase” refers to an activity exhibited by many organisms including humans that disassembles or disassembles and proteolyzes protein aggregates, for example, amyloid proteins or their precursors.

“Unfolded protein response (UPR) pathway” refers to a stress sensing mechanism in the endoplasmic reticulum (ER) wherein the ER responds to the accumulation of unfolded proteins in its lumen by activating up to three integrated arms of intracellular signaling pathways, e.g., UPR-associated stress sensors, IRE1, ATF6, and PERK, collectively referred to as the unfolded protein response, that regulate the expression of numerous genes that function within the secretory pathway. Ron et al., Nat Rev Mol Cell Biol 8: 519-529, 2007; Schroeder et al., Ann Rev Biochem 74: 739-789, 2005. UPR associated chaperones include, but are not limited to BiP, GRP94, and calreticulin.

“Heat shock response (HSR) pathway” refers to enhanced expression of heat shock proteins (chaperone/cochaperone/folding enzymes) in the cytosol that can have an effect on proteostasis of proteins folded and trafficked within the secretory pathway as a soluble lumenal enzyme. Cytosolic factors including chaperones are likely essential for adapting the secretory pathway to be more folding and trafficking permissive. Bush et al., J Biol Chem 272: 9086-9092, 1997; Liao et al., J Cell Biochem 99: 1085-1095, 2006; Westerheide et al., J Biol Chem 279: 56053-56060, 2004.

HSR-associated chaperones include, but are not limited to Hsp/c40 family members, Hsp/c70 family members, Hsp/c90 family members, the Hsp/c 40/70/90 cochaperones including Aha1, auxilin, Bag1, CSP, as well as the small heat shock protein family members. The HSR pathway also directly influences the proteome residing and functioning in the cytoplasm.”

UPR-associated chaperones include, but are not limited to, GRP78/BiP, GRP94/gp96, GRP170/ORP150, GRP58/ERp57, PDI, ERp72, calnexin, calreticulin, EDEM, Herp and co-chaperones SIL1 and P58IPK.

“Folding enzymes” refer to proteins that catalyze the slow steps in folding including, but not limited to, disulfide bond formation by protein disulfide isomerase(PDI) and peptidyl-prolyl cis-trans-amide bond isomerization by peptidyl prolyl cis-trans isomerase (PPI).

“Treating” or “treatment” includes the administration of the compositions, compounds or agents of aspects of the present invention to prevent or delay the onset of the symptoms, complications, or biochemical indicia of a disease, alleviating or ameliorating the symptoms or arresting or inhibiting further development of the disease, condition, or disorder (for example, a gain of function disorder or disease related to the accumulation of toxic aggregates, for example, Alzheimer's disease, Huntington's disease, age-related macular degeneration, inclusion body myositosis, and Parkinson's disease; or a loss of function disorder, for example, a lysosomal storage disease, cystic fibrosis, or α1-antitrypsin deficiency-associated emphysema). As used herein, the phrases “reducing a condition” or “to reduce a condition” or “reducing a disease” or “to reduce a disease” encompass ameliorating one or more symptoms of the condition or disease. The phrases “eliminating a condition” or “to eliminate a condition” or “eliminating a disease” or “to eliminate a disease” refer to ameliorating all or substantially all of the symptoms of the condition or disease. “Treating” further refers to any indicia of success in the treatment or amelioration or prevention of the disease, condition, or disorder (e.g., a gain of function disorder or disease related to the accumulation of toxic protein aggregates or a loss of function disorder, e.g., a lysosomal storage disease), including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of aspects of the present invention to prevent or delay, to alleviate, or to arrest or inhibit development of the symptoms or conditions associated with a gain of function disorder or disease related to the accumulation of toxic aggregates or a loss of function disorder, e.g., a lysosomal storage disease. The term “therapeutic effect” refers to the reduction, elimination, or prevention of the disease, symptoms of the disease, or side effects of the disease in the subject. “Treating” or “treatment” using the methods of the present invention includes preventing the onset of symptoms in a subject that can be at increased risk of a gain of function disorder or disease related to the accumulation of toxic aggregates or a loss of function disorder, e.g., a lysosomal storage disease but does not yet experience or exhibit symptoms, inhibiting the symptoms of the disease (slowing or arresting its development), providing relief from the symptoms or side-effects of the disease (including palliative treatment), and relieving the symptoms of the degenerative disease (causing regression). Treatment can be prophylactic (to prevent or delay the onset of the disease, or to prevent the manifestation of clinical or subclinical symptoms thereof) or therapeutic suppression or alleviation of symptoms after the manifestation of the disease or condition. The dosing schedule for administering proteostasis regulators to treat a particular disease or condition will likely be less frequent than the dosing schedule for other drugs used to treat the same disease or condition.

“Patient”, “subject”, “vertebrate” or “mammal” are used interchangeably and refer to mammals such as human patients and non-human primates, as well as experimental animals such as rabbits, rats, and mice, and other animals. Animals include all vertebrates and invertebrates, e.g., mammals and non-mammals, such as sheep, dogs, cows, chickens, Cenorhabditis elegans, Drosophila melanogaster, amphibians, and reptiles.

### Loss-of-Function Diseases and Lysosomal Storage Disease

“Loss of function disease” refers to a group of diseases characterized by inefficient folding of a protein resulting in excessive degradation of the protein. Loss of function diseases include, for example, cystic fibrosis, lysosomal storage diseases, and Von Hippel-Lindau (VHL) Disease. In cystic fibrosis, the mutated or defective enzyme is the cystic fibrosis transmembrane conductance regulator (CFTR). One of the most common mutations of this protein is ΔF508 which is a deletion (Δ) of three nucleotides resulting in a loss of the amino acid phenylalanine (F) at the 508th (508) position on the protein. In one embodiment, the invention is directed to a method of treating a loss of function disease in a patient in need thereof comprising administering to said patient a proteostasis regulator in an amount effective to improve or restore activity of the mutated enzyme. In a further embodiment, the proteostasis regulator restores the activity of the mutated enzyme by promoting correct folding of the mutated enzyme.

“Lysosomal storage disease” refers to a group of diseases characterized by a specific lysosomal enzyme deficiency which may occur in a variety of tissues, resulting in the build up of molecules normally degraded by the deficient enzyme. The lysosomal enzyme deficiency can be in a lysosomal hydrolase or a protein involved in the lysosomal trafficking. Representative lysosomal diseases and defective enzymes involved are listed in Table 1.

TABLE 1

| Lysosomal storage disease | Defective enzyme |
|---|---|
| Aspartylglucosaminuria | Aspartylglucosaminidase |
| Fabry | α-Galactosidase A |
| Batten (CNL1-CNL8) | Multiple gene products |
| Cystinosis | Cysteine transporter |
| Farber | Acid ceramidase |
| Fucosidosis | Acid α-L-fucosidase |
| Galactosidosialidosis | Protective protein/cathepsin A |
| Gaucher types 1, 2, and 3 | Acid β-glucosidase, or |
| | glucocerebrosidase |
| G_{M1} gangliosidosis | Acid β-galactosidase |
| Hunter | Iduronate-2-sulfatase |
| Hurler-Scheie | α-L-Iduronidase |
| Krabbe | Galactocerebrosidase |
| α-Mannosidosis | Acid α-mannosidase |
| β-Mannosidosis | Acid β-mannosidase |
| Maroteaux-Lamy | Arylsulfatase B |
| Metachromatic | Arylsulfatase A |
| leukodystrophy | |
| Morquio A | N-Acetylgalactosamine-6-sulfate |
| | sulfatase |
| Morquio B | Acid β-galactosidase |
| Mucolipidosis II/III | N-Acetylglucosamine-1- |
| | phosphotransferase |
| Niemann-Pick A, B | Acid sphingomyelinase |
| Niemann-Pick C | NPC-1 |
| Pompe | Acid α-glucosidase |
| Sandhoff | β-Hexosaminidase B |
| Sanfilippo A | Heparan N-sulfatase |
| Sanfilippo B | α-N-Acetylglucosaminidase |
| Sanfilippo C | Acetyl-CoA: α-glucosaminide N- |
| | acetyltransferase |
| Sanfilippo D | N-Acetylglucosamine-6-sulfate |
| | sulfatase |
| Schindler Disease | α-N-Acetylgalactosaminidase |
| Schindler-Kanzaki | α-N-Acetylgalactosaminidase |
| Sialidosis | α-Neuramidase |
| Sly | β-Glucuronidase |
| Tay-Sachs | β-Hexosaminidase A |
| Wolman | Acid Lipase |

Gaucher's disease, first described by Phillipe C. E. Gaucher in 1882, is the oldest and most common lysosomal storage disease known. Type I is the most common among three recognized clinical types and follows a chronic course which does not involve the nervous system. Types 2 and 3 both have a CNS component, the former being an acute infantile form with death by age two and the latter a subacute juvenile form. The incidence of Type 1 Gaucher's disease is about one in 50,000 live births generally and about one in 400 live births among Ashkenazim. Kolodny et al., 1998, “Storage Diseases of the Reticuloendothelial System”, In: Nathan and Oski's Hematology of Infancy and Childhood, 5th ed., vol. 2, David G. Nathan and Stuart H. Orkin, Eds., W.B. Saunders Co., pages 1461-1507. Also known as glucosylceramide lipidosis, Gaucher's disease is caused by inactivation of the enzyme glucocerebrosidase and accumulation of glucocerebroside. Glucocerebrosidase normally catalyzes the hydrolysis of glucocerebroside to glucose and ceramide. In Gaucher's disease, glucocerebroside accumulates in tissue macrophages which become engorged and are typically found in liver, spleen and bone marrow and occasionally in lung, kidney and intestine. Secondary hematologic sequelae include severe anemia and thrombocytopenia in addition to the characteristic progressive hepatosplenomegaly and skeletal complications, including osteonecrosis and osteopenia with secondary pathological fractures. See, for example, U.S. Application No. 2007/0280925.

Fabry disease is an X-linked recessive LSD characterized by a deficiency of α-galactosidase A (α-Gal A), also known as ceramide trihexosidase, which leads to vascular and other disease manifestations via accumulation of glycosphingolipids with terminal α-galactosyl residues, such as globotriaosylceramide (GL-3). Desnick R J et al., The Metabolic and Molecular Bases of Inherited Disease 7: 2741-2784, 1995. Symptoms may include anhidrosis (absence of sweating), painful fingers, left ventricular hypertrophy, renal manifestations, and ischemic strokes. The severity of symptoms varies dramatically. Grewal, J. Neurol. 241: 153-156, 1994. A variant with manifestations limited to the heart is recognized, and its incidence may be more prevalent than once believed. Nakao, N. Engl. J. Med. 333: 288-293, 1995. Recognition of unusual variants can be delayed until quite late in life, although diagnosis in childhood is possible with clinical vigilance. Ko et al., Arch. Pathol. Lab. Med. 120: 86-89, 1996; Mendez et al., Dement. Geriatr. Cogn. Disord. 8: 252-257, 1997; Shelley et al., Pediatric Derm. 12: 215-219, 1995. The mean age of diagnosis of Fabry disease is 29 years.

Niemann-Pick disease, also known as sphingomyelin lipidosis, comprises a group of disorders characterized by foam cell infiltration of the reticuloendothelial system. Foam cells in Niemann-Pick become engorged with sphingomyelin and, to a lesser extent, other membrane lipids including cholesterol. Niemann-Pick is caused by inactivation of the enzyme sphingomyelinase in Types A and B disease, with 27-fold more residual enzyme activity in Type B. Kolodny et al., 1998, Id. The pathophysiology of major organ systems in Niemann-Pick can be briefly summarized as follows. The spleen is the most extensively involved organ of Type A and B patients. The lungs are involved to a variable extent, and lung pathology in Type B patients is the major cause of mortality due to chronic bronchopneumonia. Liver involvement is variable, but severely affected patients may have life-threatening cirrhosis, portal hypertension, and ascites. The involvement of the lymph nodes is variable depending on the severity of disease. Central nervous system (CNS) involvement differentiates the major types of Niemann-Pick. While most Type B patients do not experience CNS involvement, it is characteristic in Type A patients. The kidneys are only moderately involved in Niemann Pick disease.

The mucopolysaccharidoses (MPS) comprise a group of LSDs caused by deficiency of enzymes which catalyze the degradation of specific glycosaminoglycans (mucopolysaccharides or GAGs) known as dermatan sulfate and heparan sulfate. GAGs contain long unbranched polysaccharides characterized by a repeating disaccharide unit and are found in the body linked to core proteins to form proteoglycans. Proteoglycans are located primarily in the extracellular matrix and on the surface of cells where they lubricate joints and contribute to structural integrity. Neufeld et al., The Metabolic and Molecular Bases of Inherited Diseases 7: 2465-2494, 1995.

The several mucopolysaccharidoses are distinguished by the particular enzyme affected in GAG degradation. For example, MPS I (Hurler-Scheie) is caused by a deficiency of α-L-iduronidase which hydrolyzes the terminal α-L-iduronic acid residues of dermatan sulfate. Symptoms in MPS I vary along a clinical continuum from mild (MPS IS or Scheie disease) to intermediate (MPS IHS or Hurler-Scheie disease) to severe (MPS IH or Hurler disease), and the clinical presentation correlates with the degree of residual enzyme activity. The mean age at diagnosis for Hurler syndrome is about nine months, and the first presenting symptoms are often among the following: coarse facial features, skeletal abnormalities, clumsiness, stiffness, infections and hernias. Cleary et al., Acta. Paediatr. 84: 337-339, 1995; Colville et al., Child: Care, Health and Development 22: 31-361996, 1996.

Other examples of mucopolysaccharidoses include Hunter (MPS II or iduronate sulfatase deficiency), Morquio (MPS IV; deficiency of galactosamine-6-sulfatase and β-galactosidase in types A and B, respectively) and Maroteaux-Lamy (MPS VI or arylsulfatase B deficiency). Neufeld et al., 1995, Id.; Kolodny et al., 1998, Id.

Pompe disease (also known as glycogen storage disease type II, acid maltase deficiency and glycogenosis type II) is an autosomal recessive LSD characterized by a deficiency of α-glucosidase (also known as acid α-glucosidase and acid maltase). The enzyme α-glucosidase normally participates in the degradation of glycogen to glucose in lysosomes; it can also degrade maltose. Hirschhorn, The Metabolic and Molecular Bases of Inherited Disease 7: 2443-2464, 1995. The three recognized clinical forms of Pompe disease (infantile, juvenile and adult) are correlated with the level of residual α-glucosidase activity. Reuser et al., Muscle & Nerve Supplement 3: S61-S69, 1995.

Infantile Pompe disease (type I or A) is most common and most severe, characterized by failure to thrive, generalized hypotonia, cardiac hypertrophy, and cardiorespiratory failure within the second year of life. Juvenile Pompe disease (type II or B) is intermediate in severity and is characterized by a predominance of muscular symptoms without cardiomegaly. Juvenile Pompe individuals usually die before reaching 20 years of age due to respiratory failure. Adult Pompe disease (type III or C) often presents as a slowly progressive myopathy in the teenage years or as late as the sixth decade. Felice et al., Medicine 74: 131-135, 1995.

In Pompe, it has been shown that α-glucosidase is extensively modified post-translationally by glycosylation, phosphorylation, and proteolytic processing. Conversion of the 110 kilodalton (kDa) precursor to 76 and 70 kDa mature forms by proteolysis in the lysosome is required for optimum glycogen catalysis.

α-1 antitrypsin associated emphysema is one of the most common inherited diseases in the Caucasian population. The most common symptom is lung disease (emphysema). People with α-1 antitrypsin disease may also develop liver disease and/or liver cancer. The disease is caused by a deficiency in the protein alpha-1 antitrypsin, The development of lung disease is accelerated by harmful environmental exposures, such as smoking tobacco. α-1 antitrypsin disease has a genetic component. The age of onset, rate of progression, and type of symptoms vary both between and within families.

von Hippel-Lindau disease (VHL) is a rare, genetic multi-system disorder characterized by the abnormal growth of tumors in certain parts of the body (angiomatosis). The tumors of the central nervous system (CNS) are benign and are comprised of a nest of blood vessels (hemangioblastomas). Hemangioblastomas may develop in the brain, the retina of the eyes, and other areas of the nervous system. Other types of tumors develop in the adrenal glands, the kidneys, or the pancreas. Individuals with VHL are also at a higher risk than normal for certain types of cancer, especially kidney cancer. In the case of VHL, proteostasis regulators can restore enzyme function indirectly. Methods for treating a loss of function disease in a patient in need thereof comprising administering a proteostasis regulator in an amount effective to improve or restore activity of a protein, for example, the mutated VHL protein (PVHL) that serves as an adaptor for enzymes. Misfolding of pVHL compromises the ability of enzymes to target the hypoxia-inducible transcription factor (HIF) for polyubiquitylation and proteasomal degradation, leading to cancer. Proteostasis regulators can restore enzyme function indirectly to treat disease such as VHL disease.

Hereditary spastic paraplegias (HSPs) are characterized by progressive lower limb spasticity and weakness. Mutations in the SPG3A gene, which encodes the large guanosine triphosphatase atlastin enzyme, are the second most common cause of autosomal dominant hereditary spastic paraplegia. In a large SPG3A screen of 70 hereditary spastic paraplegia subjects, a novel in-frame deletion, p.del436N, was identified. Characterization of this deletion showed that it affects neither the guanosine triphosphatase activity of atlastin nor interactions between atlastin and spastin. Interestingly, immunoblot analysis of lymphoblasts from affected patients demonstrated a significant reduction in atlastin protein levels, supporting a loss-of-function disease mechanism. Annals of Neurology 61(6): 599-603, 2007

The gene underlying Marinesco-Sjogren syndrome has been identified. Marinesco-Sjogren syndrome is characterized by cerebellar ataxia, progressive myopathy and cataracts. Four disease-associated predicted loss-of-function mutations in SIL1 were identified. SIL1 encodes a nucleotide exchange factor enzyme for the heat-shock protein 70 (HSP70) chaperone HSPA5. These data, together with the similar spatial and temporal patterns of tissue expression of Sil1 and Hspa5, suggest that disturbed SIL1-HSPA5 interaction and protein folding is the primary pathology in Marinesco-Sjogren syndrome. Nature Genetics 37(12): 1309-1311, 2005.

Autosomal dominant hypertrophic cardiomyopathy (HCM) is caused by inherited defects of sarcomeric proteins. The hypothesis was tested that homozygosity for a sarcomeric protein defect can cause recessive HCM. A family was studied with early-onset cardiomyopathy in 3 siblings, characterized by mid-cavitary hypertrophy and restrictive physiology. Genotyping of DNA markers spanning 8 genes for autosomal dominant HCM revealed inheritance of an identical paternal and maternal haplotype at the essential light chain of myosin locus by the affected children. Sequencing showed that these individuals were homozygous for a Glu143Lys substitution of a highly conserved amino acid that was absent in 150 controls. Family members with one Glu143Lys allele had normal echocardiograms and ECGs, even in late adulthood, whereas those with two mutant alleles developed severe cardiomyopathy in childhood. These findings, coupled with previous studies, of myosin light chain structure and function in the heart, suggest a loss-of-function disease mechanism. Distinct mutations affecting the same sarcomeric protein can cause either dominant or recessive cardiomyopathy. Electrostatic charge reversal of a highly conserved amino acid may be benign in the heterozygous state as the result of compensatory mechanisms that preserve cardiac structure and function. By contrast, homozygous carriers of a sarcomeric, protein defect, may have a malignant course. Circulation 105(20): 2337-2340, 2002.

### Gain of Function Diseases

A “gain of function disease” refers to a disease characterized by increased aggregation-associated proteotoxicity. In these diseases, aggregation exceeds clearance inside and/or outside of the cell. Gain of function diseases are often associated with aging and are also referred to as “gain of toxic function” diseases. In one embodiment, the invention is directed to a method of treating a gain of function disease in a patient in need thereof comprising administering to said patient a proteostasis regulator in an amount effective to decrease aggregation of the protein. In a further embodiment, the proteostasis regulator decreases aggregation of the protein by promoting correct folding of the protein, inhibiting an aggregase pathway or stimulating the activity of a disaggregase. In a further embodiment, the proteostasis regulator would influence aggregation in a fashion that would decrease cytotoxicity.

Gain of function diseases include, but are not limited to neurodegenerative disease associated aggregation of polyglutamine repeats in proteins or repeats at other amino acids such as alanine, Lewy body diseases and other disorders associated with α-synuclein aggregation, amyotrophic lateral sclerosis, transthyretin-associated aggregation diseases, Alzheimer's disease, age-associated macular degeneration, inclusion body myositosis, and prion diseases. Neurodegenerative diseases associated with aggregation of polyglutamine include, but are not limited to, Huntington's disease, dentatorubral and pallidoluysian atrophy, several forms of spino-cerebellar ataxia, and spinal and bulbar muscular atrophy. Alzheimer's disease is characterized by the formation of two types of aggregates: intracellular and extracellular aggregates of Aβ peptide and intracellular aggregates of the microtubule associated protein tau. Transthyretin-associated aggregation diseases include, for example, senile systemic amyloidoses, familial amyloidotic neuropathy, and familial amyloid cardiomyopathy. Lewy body diseases are characterized by an aggregation of α-synuclein protein and include, for example, Parkinson's disease. Prion diseases (also known as transmissible spongiform encephalopathies) are characterized by aggregation of prion proteins. Exemplary human prion diseases are Creutzfeldt-Jakob Disease (CJD), Variant Creutzfeldt-Jakob Disease, Gerstmann-Straussler-Scheinker Syndrome. Fatal Familial Insomnia and Kuru.

Molecular disorders of G proteins and signal transduction can result in gain of function disease or loss of function disease. Gain of function type diseases are caused by hyperactivity of Gα by suppression of GTPase activity. Mutations in αs gene (gsp) and αi (gip2) generate endocrine tumors, and anomalous expression of gsp generates McCune-Albright syndrome and growth hormone-secreting pituitary adenoma. Gain-and-loss-of-function disease by AS mutation, i.e., Ala366 to Ser in αs (αs-A366S) shows testotoxicosis and pseudohypoparathyroidism type Ia accompanying Albright hereditary osteodystrophy. The αs-A366S exhibits dominant-positive effects and dominant-negative effects. The αs-A366S mimics activation of Gs by the receptor, and exhibits temperature-sensitive features. Various modes of the loss-of-function of αs have been identified and lead to a mechanism of the dominant-negative effects. Jikken Igaku 14(2): 219-224, 1996.

### RNA and DNA Interference Methods

A. Short Interfering RNA (RNAi)

RNA interference (RNAi) is a mechanism of post-transcriptional gene silencing mediated by double-stranded RNA (dsRNA), which is distinct from antisense and ribozyme-based approaches. Jain, Pharmacogenomics 5: 239-42, 2004. RNA interference is useful in a method for treating a condition characterized by dysfunction in protein homeostasis in a patient in need thereof by administering to the patient a proteostasis regulator in an amount effective to improve or restore protein homeostasis, and to reduce or eliminate the condition in the patient or to prevent its occurrence or recurrence. dsRNA molecules are believed to direct sequence-specific degradation of mRNA in cells of various types after first undergoing processing by an RNase III-like enzyme called DICER into smaller dsRNA molecules comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs. Bernstein et al., Nature 409: 363, 2001. RNAi is thus mediated by short interfering RNAs (siRNA), which typically comprise a double-stranded region approximately 19 nucleotides in length with 1-2 nucleotide 3′ overhangs on each strand, resulting in a total length of between approximately 21 and 23 nucleotides. In mammalian cells, dsRNA longer than approximately 30 nucleotides typically induces nonspecific mRNA degradation via the interferon response. However, the presence of siRNA in mammalian cells, rather than inducing the interferon response, results in sequence-specific gene silencing.

In general, a short, interfering RNA (siRNA) comprises an RNA duplex that is preferably approximately 19 basepairs long and optionally further comprises one or two single-stranded overhangs or loops. An siRNA may comprise two RNA strands hybridized together, or may alternatively comprise a single RNA strand that includes a self-hybridizing portion. siRNAs may include one or more free strand ends, which may include phosphate and/or hydroxyl groups. siRNAs typically include a portion that hybridizes under stringent conditions with a target transcript. One strand of the siRNA (or, the self-hybridizing portion of the siRNA) is typically precisely complementary with a region of the target transcript, meaning that the siRNA hybridizes to the target transcript without a single mismatch. In certain embodiments of the invention in which perfect complementarity is not achieved, it is generally preferred that any mismatches be located at or near the siRNA termini.

siRNAs have been shown to downregulate gene expression when transferred into mammalian cells by such methods as transfection, electroporation, or microinjection, or when expressed in cells via any of a variety of plasmid-based approaches. RNA interference using siRNA is reviewed in, e.g., Tuschl, Nat. Biotechnol. 20: 446-448, 2002; See also Yu, J., et al., Proc. Natl. Acad. Sci., 99: 6047-6052, 2002; Sui, et al., Proc. Natl. Acad. Sci. USA. 99: 5515-5520, 2002; Paddison, et al., Genes and Dev. 16: 948-958, 2002; Brummelkamp, et al., Science 296: 550-553, 2002; Miyagashi, et al., Nat. Biotech. 20: 497-500, 2002; Paul, et al., Nat. Biotech. 20: 505-508, 2002. As described in these and other references, the siRNA may consist of two individual nucleic acid strands or of a single strand with a self-complementary region capable of forming a hairpin (stem-loop) structure. A number of variations in structure, length, number of mismatches, size of loop, identity of nucleotides in overhangs, etc., are consistent with effective siRNA-triggered gene silencing. While not wishing to be bound by any theory, it is thought that intracellular processing (e.g., by DICER) of a variety of different precursors results in production of siRNA capable of effectively mediating gene silencing. Generally it is preferred to target exons rather than introns, and it may also be preferable to select sequences complementary to regions within the 3′ portion of the target transcript. Generally it is preferred to select sequences that contain approximately equimolar ratio of the different nucleotides and to avoid stretches in which a single residue is repeated multiple times. P siRNAs may thus comprise RNA molecules having a double-stranded region approximately 19 nucleotides in length with 1-2 nucleotide 3′ overhangs on each strand, resulting in a total length of between approximately 21 and 23 nucleotides. As used herein, siRNAs also include various RNA structures that may be processed in vivo to generate such molecules. Such structures include RNA strands containing two complementary elements that hybridize to one another to form a stem, a loop, and optionally an overhang, preferably a 3′ overhang. Preferably, the stem is approximately 19 bp long, the loop is about 1-20, more preferably about 4-10, and most preferably about 6-8 nt long and/or the overhang is about 1-20, and more preferably about 2-15 nt long. In certain embodiments of the invention the stem is minimally 19 nucleotides in length and may be up to approximately 29 nucleotides in length. Loops of 4 nucleotides or greater are less likely subject to steric constraints than are shorter loops and therefore may be preferred. The overhang may include a 5′ phosphate and a 3′ hydroxyl. The overhang may but need not comprise a plurality of U residues, e.g., between 1 and 5 U residues. Classical siRNAs as described above trigger degradation of mRNAs to which they are targeted, thereby also reducing the rate of protein synthesis. In addition to siRNAs that act via the classical pathway, certain siRNAs that bind to the 3′ UTR of a template transcript may inhibit expression of a protein encoded by the template transcript by a mechanism related to but distinct from classic RNA interference, e.g., by reducing translation of the transcript rather than decreasing its stability. Such RNAs are referred to as microRNAs (mRNAs) and are typically between approximately 20 and 26 nucleotides in length, e.g., 22 nt in length. It is believed that they are derived from larger precursors known as small temporal RNAs (stRNAs) or mRNA precursors, which are typically approximately 70 nt long with an approximately 4-15 nt loop. Grishok, et al., Cell 106: 23-24, 2001; Hutvagner, et al., Science 293: 834-838, 2001; Ketting, et al., Genes Dev., 15: 2654-2659, 2001. Endogenous RNAs of this type have been identified in a number of organisms including mammals, suggesting that this mechanism of post-transcriptional gene silencing may be widespread. Lagos-Quintana, et al, Science 294: 853-858, 2001; Pasquinelli, Trends in Genetics 18: 171-173, 2002, and references in the foregoing two articles. MicroRNAs have been shown to block translation of target transcripts containing target sites in mammalian cells. Zeng, et al, Molecular Cell 9: 1-20, 2002.

siRNAs such as naturally occurring or artificial (i.e., designed by humans) mRNAs that bind within the 3′ UTR (or elsewhere in a target transcript) and inhibit translation may tolerate a larger number of mismatches in the siRNA/template duplex, and particularly may tolerate mismatches within the central region of the duplex. In fact, there is evidence that some mismatches may be desirable or required as naturally occurring stRNAs frequently exhibit such mismatches as do mRNAs that have been shown to inhibit translation in vitro. For example, when hybridized with the target transcript such siRNAs frequently include two stretches of perfect complementarity separated by a region of mismatch. A variety of structures are possible. For example, the mRNA may include multiple areas of nonidentity (mismatch). The areas of nonidentity (mismatch) need not be symmetrical in the sense that both the target and the mRNA include nonpaired nucleotides. Typically the stretches of perfect complementarity are at least 5 nucleotides in length, e.g., 6, 7, or more nucleotides in length, while the regions of mismatch may be, for example, 1, 2, 3, or 4 nucleotides in length.

Hairpin structures designed to mimic siRNAs and mRNA precursors are processed intracellularly into molecules capable of reducing or inhibiting expression of target transcripts. McManus, et al., RNA 8: 842-850, 2002. These hairpin structures, which are based on classical siRNAs consisting of two RNA strands forming a 19 bp duplex structure are classified as class I or class II hairpins. Class I hairpins incorporate a loop at the 5′ or 3′ end of the antisense siRNA strand (i.e., the strand complementary to the target transcript whose inhibition is desired) but are otherwise identical to classical siRNAs. Class II hairpins resemble mRNA precursors in that they include a 19 nt duplex region and a loop at either the 3′ or 5′ end of the antisense strand of the duplex in addition to one or more nucleotide mismatches in the stem. These molecules are processed intracellularly into small RNA duplex structures capable of mediating silencing. They appear to exert their effects through degradation of the target mRNA rather than through translational repression as is thought to be the case for naturally occurring mRNAs and stRNAs.

Thus it is evident that a diverse set of RNA molecules containing duplex structures is able to mediate silencing through various mechanisms. For the purposes of the present invention, any such RNA, one portion of which binds to a target transcript and reduces its expression, whether by triggering degradation, by inhibiting translation, or by other means, is considered to be an siRNA, and any structure that generates such an siRNA (i.e., serves as a precursor to the RNA) is useful in the practice of the present invention.

In the context of the present invention, siRNAs are useful both for therapeutic purposes, e.g., to act as a proteostasis regulator in an amount effective to improve or restore protein homeostasis in a patient in need thereof and for various of the inventive methods for the identification of compounds for treatment of a condition characterized by dysfunction in protein homeostasis in a patient in need thereof. In a preferred embodiment, the therapeutic treatment with an antibody, antisense vector, or double stranded RNA vector is useful for a loss of function disorder, e.g., a lysosomal storage disease, or a gain of function disorder with an antibody, antisense vector, or double stranded RNA vector.

The invention therefore provides a method for treating a condition characterized by dysfunction in protein homeostasis in a patient in need thereof which comprises administering to the patient a proteostasis regulator in an amount effective to improve or restore protein homeostasis, and to reduce or eliminate the condition in the patient or to prevent its occurrence or recurrence, wherein the proteostasis regulator is an siRNA. The proteostasis regulator can upregulate signaling via a heat shock response (HSR) pathway, an unfolded protein response (UPR) pathway, and/or a Ca²⁺ signaling pathway. According to certain embodiments of the invention the biological system comprises a cell, and the contacting step comprises expressing the siRNA in the cell. According to certain embodiments of the invention the biological system comprises a subject, e.g., a mammalian subject such as a mouse or human, and the contacting step comprises administering the siRNA to the subject or comprises expressing the siRNA in the subject. According to certain embodiments of the invention the siRNA is expressed inducibly and/or in a cell-type or tissue specific manner.

By “biological system” is meant any vessel, well, or container in which biomolecules (e.g., nucleic acids, polypeptides, polysaccharides, lipids, and the like) are placed; a cell or population of cells; a tissue; an organ; an organism, and the like. Typically the biological system is a cell or population of cells, but the method can also be performed in a vessel using purified or recombinant proteins.

The invention provides siRNA molecules targeted to a gene or gene product to provide upregulated signaling via a heat shock response (HSR) pathway, an unfolded protein response (UPR) pathway, and/or a Ca²⁺ signaling pathway. In particular, the invention provides siRNA molecules selectively or specifically targeted to a transcript encoding a polymorphic variant of such a transcript, wherein existence of the polymorphic variant in a subject is indicative of susceptibility to or presence of a condition characterized by dysfunction in protein homeostasis. The terms “selectively” or “specifically targeted to”, in this context, are intended to indicate that the siRNA causes greater reduction in expression of the variant than of other variants (i.e., variants whose existence in a subject is not indicative of susceptibility to or presence of a loss of function disorder, e.g., a lysosomal storage disease, or a gain of function disorder). The siRNA, or collections of siRNAs, may be provided in the form of kits with additional components as appropriate.

B. Short Hairpin RNAs (shRNA)

RNA interference (RNAi), a mechanism of post-transcriptional gene silencing mediated by double-stranded RNA (dsRNA), is useful in a method for treatment of a condition characterized by dysfunction in protein homeostasis in a patient in need thereof by administering a nucleic acid molecule (e.g., dsRNA) that hybridizes under stringent conditions to a target gene, and attenuates expression of said target gene. See Jain, Pharmacogenomics 5: 239-42, 2004 for a review of RNAi and siRNA. A further method of RNA interference in the present invention is the use of short hairpin RNAs (shRNA). A plasmid containing a DNA sequence encoding for a particular desired siRNA sequence is delivered into a target cell via transfection or virally-mediated infection. Once in the cell, the DNA sequence is continuously transcribed into RNA molecules that loop back on themselves and form hairpin structures through intramolecular base pairing. These hairpin structures, once processed by the cell, are equivalent to transfected siRNA molecules and are used by the cell to mediate RNAi of the desired protein. The use of shRNA has an advantage over siRNA transfection as the former can lead to stable, long-term inhibition of protein expression. Inhibition of protein expression by transfected siRNAs is a transient phenomenon that does not occur for times periods longer than several days. In some cases, this may be preferable and desired. In cases where longer periods of protein inhibition are necessary, shRNA mediated inhibition is preferable.

C. Full and Partial Length Antisense RNA Transcripts

Antisense RNA transcripts have a base sequence complementary to part or all of any other RNA transcript in the same cell. Such transcripts have been shown to modulate gene expression through a variety of mechanisms including the modulation of RNA splicing, the modulation of RNA transport and the modulation of the translation of mRNA. Denhardt, Ann N Y Acad. Sci. 660: 70, 1992; Nellen, Trends Biochem. Sci. 18: 419, 1993; Baker et al, Biochim. Biophys. Acta, 1489: 3, 1999; Xu, et al., Gene Therapy 7: 438, 2000; French et al., Curr. Opin. Microbiol. 3: 159, 2000; Terryn et al., Trends Plant Sci. 5: 1360, 2000.

D. Antisense RNA and DNA Oligonucleotides

Antisense nucleic acids are generally single-stranded nucleic acids (DNA, RNA, modified DNA, or modified RNA) complementary to a portion of a target nucleic acid (e.g., an mRNA transcript) and therefore able to bind to the target to form a duplex. Typically they are oligonucleotides that range from 15 to 35 nucleotides in length but may range from 10 up to approximately 50 nucleotides in length. Binding typically reduces or inhibits the function of the target nucleic acid. For example, antisense oligonucleotides may block transcription when bound to genomic DNA, inhibit translation when bound to mRNA, and/or lead to degradation of the nucleic acid. Reduction in expression of a target polypeptide for treatment of a condition characterized by dysfunction in protein homeostasis may be achieved by the administration of antisense nucleic acids or peptide nucleic acids comprising sequences complementary to those of the mRNA that encodes the polypeptide. Antisense technology and its applications are well known in the art and are described in Phillips, M. I. (ed.) Antisense Technology, Methods Enzymol., 2000, Volumes 313 and 314, Academic Press, San Diego, and references mentioned therein. See also Crooke, S. (ed.) “ANTISENSE DRUG TECHNOLOGY: PRINCIPLES, STRATEGIES, AND APPLICATIONS” (1^{st} Edition) Marcel Dekker; and references cited therein.

Antisense oligonucleotides can be synthesized with a base sequence that is complementary to a portion of any RNA transcript in the cell. Antisense oligonucleotides may modulate gene expression through a variety of mechanisms including the modulation of RNA splicing, the modulation of RNA transport and the modulation of the translation of mRNA (Denhardt, 1992). Various properties of antisense oligonucleotides including stability, toxicity, tissue distribution, and cellular uptake and binding affinity may be altered through chemical modifications including (i) replacement of the phosphodiester backbone (e.g., peptide nucleic acid, phosphorothioate oligonucleotides, and phosphoramidate oligonucleotides), (ii) modification of the sugar base (e.g., 2′-O-propylribose and 2′-methoxyethoxyribose), and (iii) modification of the nucleoside (e.g., C-5 propynyl U, C-5 thiazole U, and phenoxazine C). Wagner, Nat. Medicine 1: 1116, 1995; Varga, et al., Immun. Lett. 69: 217, 1999; Neilsen, Curr. Opin. Biotech. 10: 71, 1999; Woolf, Nucleic Acids Res. 18: 1763, 1990.

The invention therefore provides a method for treating a condition characterized by dysfunction in protein homeostasis in a patient in need thereof which comprises administering to the patient a proteostasis regulator in an amount effective to improve or restore protein homeostasis, and to reduce or eliminate the condition in the patient or to prevent its occurrence or recurrence, wherein the proteostasis regulator is an antisense molecule. According to certain embodiments of the invention the biological system comprises a cell, and the contacting step comprises expressing the siRNA in the cell. According to certain embodiments of the invention the biological system comprises a subject, e.g., a mammalian subject such as a mouse or human, and the contacting step comprises administering the siRNA to the subject or comprises expressing the siRNA in the subject. According to certain embodiments of the invention the siRNA is expressed inducibly and/or in a cell-type or tissue specific manner.

E. Ribozymes

Certain nucleic acid molecules referred to as ribozymes or deoxyribozymes have been shown to catalyze the sequence-specific cleavage of RNA molecules. The cleavage site is determined by complementary pairing of nucleotides in the RNA or DNA enzyme with nucleotides in the target RNA. Thus, RNA and DNA enzymes can be designed to cleave to any RNA molecule, thereby increasing its rate of degradation. Cotten et al, EMBO J. 8: 3861-3866, 1989; Usman et al., Nucl. Acids Mol. Biol. 10: 243, 1996; Usman, et al., Curr. Opin. Struct. Biol. 1: 527, 1996; Sun, et al., Pharmacol. Rev., 52: 325, 2000. See also e.g., Cotten et al, EMBO J. 8: 3861-3866, 1989.

The invention therefore provides a method for treating a condition characterized by dysfunction in protein homeostasis in a patient in need thereof which comprises administering to the patient a proteostasis regulator in an amount effective to improve or restore protein homeostasis, and to reduce or eliminate the condition in the patient or to prevent its occurrence or recurrence, wherein the proteostasis regulator is an antisense molecule. According to certain embodiments of the invention the biological system comprises a cell, and the contacting step comprises expressing the siRNA in the cell. According to certain embodiments of the invention the biological system comprises a subject, e.g., a mammalian subject such as a mouse or human, and the contacting step comprises administering the siRNA to the subject or comprises expressing the siRNA in the subject. According to certain embodiments of the invention the siRNA is expressed inducibly and/or in a cell-type or tissue specific manner.

### High Throughput Assays for Proteostasis Regulators

The compounds tested as proteostasis regulators which can upregulate signaling via a heat shock response (HSR) pathway, an unfolded protein response (UPR) pathway, and/or a Ca²⁺ signaling pathway can be any small organic molecule, or a biological entity, such as a protein, e.g., an antibody or peptide, a sugar, a nucleic acid, e.g., an antisense oligonucleotide, RNAi, or a ribozyme, or a lipid. Typically, test compounds will be small organic molecules, peptides, lipids, and lipid analogs.

Cell-based assays can be used for high-throughput assays for proteostasis regulators. Patient-derived cells can be used to screen a compound library for proteostasis regulators by screening for compounds that remedy either the loss of function (by measuring the function of the protein) or gain of function (by assessing ameliorated proteotoxicity or lessened aggregation) in the patient-derived cells.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries. U.S. Pat. No. 5,010,175, Furka, Int. J. Pept. Prot. Res. 37: 487-493, 1991 and Houghton et al., Nature 354: 84-88, 1991. Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90: 6909-6913, 1993), vinylogous polypeptides (Hagihara et al, J. Amer. Chem. Soc. 114: 6568, 1992), nonpeptidyl peptidomimetics with glucose scaffolding (Hirschmann et al, J. Amer. Chem. Soc. 114: 9217-9218, 1992), analogous organic syntheses of small compound libraries (Chen et al., J. Amer. Chem. Soc. 116: 2661, 1994), oligocarbamates (Cho et al, Science 261: 1303, 1993), and/or peptidyl phosphonates (Campbell et al., J. Org. Chem. 59: 658, 1994), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14: 309-314, 1996 and PCT/US96/10287), carbohydrate libraries (see, e.g., Liang et al., Science 274: 1520-1522, 1996 and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Candidate compounds are useful as part of a strategy to identify drugs for treating disorders including, but not limited to, a loss of function disorder, e.g., a lysosomal storage disease, or a gain of function disorder. A test compound that acts as a proteostasis regulator to upregulate signaling via a heat shock response (HSR) pathway, an unfolded protein response (UPR) pathway, a Ca²⁺ signaling pathway, and/or longevity pathways is considered a candidate compound.

Screening assays for identifying candidate or test compounds that act as a proteostasis regulator to upregulate signaling via a heat shock response (HSR) pathway, an unfolded protein response (UPR) pathway, and/or a Ca²⁺ signaling pathway are also included in the invention. The test compounds can be obtained using any of the numerous approaches in combinatorial library methods known in the art, including, but not limited to, biological libraries; spatially addressable parallel solid phase or solution phase libraries; synthetic library methods requiring deconvolution; the “one-bead one-compound” library method; and synthetic library methods using affinity chromatography selection. The biological library approach can be used for, e.g., peptide libraries, while the other four approaches are applicable to peptide, non-peptide oligomer or small chemical molecule libraries of compounds. Lam, Anticancer Drug Des. 12: 145, 1997). Examples of methods for the synthesis of molecular libraries can be found in the art, for example in: DeWitt et al., Proc. Natl. Acad. Sci. U.S.A. 90: 6909, 1993; Erb et al., Proc. Natl. Acad. Sci. USA 91: 11422, 1994; Zuckermann et al., J. Med. Chem. 37: 2678, 1994; Cho et al., Science 261: 1303, 1993; Carrell et al., Angew. Chem. Int. Ed. Engl. 33: 2059, 1994; Carell et al., Angew. Chem. Int. Ed. Engl. 33: 2061, 1994; and Gallop et al., J. Med. Chem. 37: 1233, 1994. In some embodiments, the test compounds are activating variants of proteostasis regulators.

Libraries of compounds can be presented in solution (e.g., Houghten, Bio/Techniques 13: 412-421, 1992), or on beads (Lam, Nature 354: 82-84, 1991), chips (Fodor, Nature 364: 555-556, 1993), bacteria (U.S. Pat. No. 5,223,409), spores (U.S. Pat. Nos. 5,571,698, 5,403,484, and 5,223,409), plasmids (Cull et al., Proc. Natl. Acad. Sci. USA 89: 1865-1869, 1992) or on phage (Scott et al., Science 249: 386-390, 1990; Devlin, Science 249: 404-406, 1990; Cwirla et al., Proc. Natl. Acad. Sci. USA 87: 6378-6382, 1990; and Felici, J. Mol. Biol. 222: 301-310, 1991).

The ability of a test compound to modulate the activity of signaling via a heat shock response (HSR) pathway, an unfolded protein response (UPR) pathway, and/or a Ca²⁺ signaling pathway a proteostasis regulator or a biologically active portion thereof can be determined, e.g., by monitoring the inhibition or activation of biological aggregation or disaggregation in cells in the presence of the test compound. Modulating the activity as a proteostasis regulator or a biologically active portion thereof can be determined by measuring biological aggregation or disaggregation in cells. The binding assays can be cell-based or cell-free.

The ability of a test compound to act as a proteostasis regulator to upregulate signaling via a heat shock response (HSR) pathway, an unfolded protein response (UPR) pathway, and/or a Ca²⁺ signaling pathway in cells can be determined by one of the methods described herein or known in the art for determining direct binding. In one embodiment, the ability of the proteostasis regulator to bind to or interact with genes or gene products involved in upregulated signaling via a heat shock response (HSR) pathway, an unfolded protein response (UPR) pathway, and/or a Ca²⁺ signaling pathway can be determined. The assay can be an aggregation or disaggregation assay. In general, such assays are used to determine the ability of a test compound to affect upregulated signaling via a heat shock response (HSR) pathway, an unfolded protein response (UPR) pathway, and/or a Ca²⁺ signaling pathway.

In general, the ability of a test compound to affect aggregation or disaggregation activity in cells is compared to a control in which the aggregation or disaggregation activity is determined in the absence of the test compound. In some cases, a predetermined reference value is used. Such reference values can be determined relative to controls, in which case a test sample that is different from the reference would indicate that the compound binds to the molecule of interest or modulates expression e.g., modulates, activates or inhibits signaling via a heat shock response (HSR) pathway, an unfolded protein response (UPR) pathway, and/or a Ca²⁺ signaling pathway. A reference value can also reflect the amount of aggregation or disaggregation with a proteostasis regulator observed with a standard (e.g., the affinity of an antibody, or modulation of the aggregation or disaggregation activity). In this case, a test compound that is similar to (e.g., equal to or less than) the reference would indicate that compound is a candidate compound (e.g., aggregation or disaggregation activity to a degree equal to or greater than a reference antibody).

This invention further pertains to novel agents identified by the above-described screening assays and uses thereof for treatments as described herein.

In one embodiment the invention provides soluble assays using proteostasis regulators, or a cell or tissue expressing genes or gene products upregulated for signaling via a heat shock response (HSR) pathway, an unfolded protein response (UPR) pathway, and/or a Ca²⁺ signaling pathway, either naturally occurring or recombinant. In another embodiment, the invention provides solid phase based in vitro assays in a high throughput format, where genes or gene products upregulated for signaling via a heat shock response (HSR) pathway, an unfolded protein response (UPR) pathway, and/or a Ca²⁺ signaling pathway is attached to a solid phase substrate via covalent or non-covalent interactions. Any one of the assays described herein can be adapted for high throughput screening.

In the high throughput assays of the invention, either soluble or solid state, it is possible to screen up to several thousand different modulators or ligands in a single day. This methodology can be used for assaying genes or gene products upregulated for signaling via a heat shock response (HSR) pathway, an unfolded protein response (UPR) pathway, and/or a Ca²⁺ signaling pathway. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as toll-like receptors, transferrin, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I, 1993. Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, polyethylene glycol linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85: 2149-2154, 1963 (describing solid phase synthesis of, e.g., peptides); Geysen et al., J. Immun. Meth. 102: 259-274, 1987 (describing synthesis of solid phase components on pins); Frank & Doring, Tetrahedron 44: 6031-6040, 1988 (describing synthesis of various peptide sequences on cellulose disks); Fodor et al., Science 251: 767-777, 1991; Sheldon et al., Clinical Chemistry 39: 718-719, 1993; and Kozal et al., Nature Medicine 2: 753-759, 1996 (all describing arrays of biopolymers fixed to solid substrates). Non-chemical approaches for fixing tag binders to substrates include other common methods, such as heat, cross-linking by UV radiation, and the like.

### Therapeutic Applications

The proteostasis regulators described herein and the proteostasis regulators identified by the methods as described herein can be used in a variety of methods for treatment of conditions characterized by dysfunction in protein homeostasis in a patient in need thereof. Thus, the present invention provides compositions and methods for treating diseases associated with a loss of function disorder, e.g., a lysosomal storage disease, or a gain of function disorder. In one embodiment, the composition includes small chemical compounds or biologics that act as a proteostasis regulator to upregulate signaling via a heat shock response (HSR) pathway, an unfolded protein response (UPR) pathway, and/or a Ca²⁺ signaling pathway, and a pharmaceutically acceptable carrier. In another embodiment, the composition comprises small chemical compounds or biologics that regulate protein chaperones by upregulating transcription or translation of the protein chaperone, or inhibiting degradation of the protein chaperone. In yet another aspect, the composition includes small chemical compounds or biologics that upregulate an aggregation pathway or a disaggregase.

The composition can be administered alone or in combination with other compositions. The proteostasis regulator composition can be administered alone or in combination with other compositions. In one aspect, the proteostasis regulator is administered in combination with a pharmacologic chaperone/kinetic stabilizer specific to the disease or condition to be treated. In another aspect, the pharmacologic chaperone/kinetic stabilizer is one that is specific to the disease or condition to be treated. A pharmacologic chaperone/kinetic stabilizer that is specific to the disease or condition to be treated is a pharmacologic chaperone/kinetic stabilizer that stabilizes the folding of a protein associated with the disease or condition and/or associated with dysfunction in homeostasis. In a further aspect, the invention is a composition comprising a proteostasis regulator and a pharmacologic chaperone/kinetic stabilizer. In yet another aspect, the invention is directed to a method of treating a condition characterized by a dysfunction in protein homeostasis in a patient in need thereof comprising administering to the patient a proteostasis regulator in combination with a pharmacologic chaperone/kinetic stabilizer wherein said combination is administered in an amount sufficient to restore homeostasis of said protein.

In an additional aspect, the invention is directed to the administration of at least two mechanistically distinct proteostasis regulators. Proteostasis regulators are mechanistically distinct if they each restore protein homeostasis of different or distinct proteins and/or modulate different proteostasis signaling pathways. Exemplary signaling pathways are the HSR, UPR and Ca²⁺ signaling pathways. In another example, as described below in the Examples, two mechanistically distinct proteostasis regulators each partially restored the folding, trafficking and function to two different mutated glycoliopid hydrolase enzymes, glucocerebrosidase and β-hexosamine A. In one aspect, one mechanistically distinct proteostasis regulator is administered with at least one other mechanistically distinct proteostasis regulator. In yet another embodiment, the invention encompasses administration of a proteostastis regulator that modulates the HSR in combination with a proteostasis regulator that modulates the UPR or a Ca²⁺ signaling pathway. In a further embodiment, the invention encompasses administration of a proteostasis regulator the UPR in combination with a proteostasis regulator that modulates the HSR or a Ca²⁺ signaling pathway. In a further aspect, the invention is directed to a method of treating a condition characterized by a dysfunction in protein homeostasis in a patient in need thereof comprising administering to the patient at least two mechanistically distinct proteostasis regulators in an amount sufficient to restore homeostasis of said protein.

The invention also encompasses a method of treating a condition characterized by a dysfunction in protein homeostasis in a patient in need thereof comprising administering to said patient a proteostasis regulator in an amount that restores homeostasis of the protein and does not increase susceptibility of the patient to viral infection. Also encompassed in the present invention is a method of treating a condition characterized by a dysfunction in protein homeostasis in a patient in need thereof comprising administering to said patient a proteostasis regulator in an amount that restores homeostasis of the protein and does not increase susceptibility of the patient to a tumor. In yet another embodiment, the proteostasis regulator does not enhance the folding of a viral protein or the synthesis of bacterial proteins. In a further embodiment, the proteostasis regulator does not enhance protein folding and trafficking capacity of tumor cells.

A proteostasis regulator composition, as described herein, can be used in methods for preventing or treating a method for treatment of a condition characterized by dysfunction in protein homeostasis in a patient in need thereof. The nature of the proteostasis regulator is of particular importance for the potential clinical usage as a factor to upregulate signaling via a heat shock response (HSR) pathway, an unfolded protein response (UPR) pathway, and/or a Ca²⁺ signaling pathway. The proteostasis regulator, e.g., a small chemical compound, thus has an unusual safety profile with minimum side effect as a survival molecule. It may therefore be used to treat a broad array of diseases related to a loss of function disorder, e.g., a lysosomal storage disease, or a gain of function disorder. The proteostasis regulator compositions therefore offers a new and better therapeutic option for the treatment of disease.

Preferably, treatment using proteostasis regulator compositions, in an aspect of the present invention, can be by administering an effective amount of the proteostasis regulator in an amount effective to improve or restore protein homeostasis in a patient in need thereof or to reduce or eliminate disease in the patient. As described above, a reduction in a disease encompasses a reduction or amelioration of one or more symptoms associated with the disease. Moreover, the proteostasis regulator compositions as provided herein can be used to reduce or eliminate a loss of function disorder, e.g., a lysosomal storage disease, or a gain of function disorder.

The invention is directed to methods of treating conditions associated with a dysfunction in protein homeostasis comprising administering to a patient a proteostasis regulator in an amount effective to improve or restore protein homeostasis. In one aspect of the invention, the condition associated with a dysfunction in the homeostasis of a protein selected from the group consisting of glucocerebrosidase, hexosamine A, cystic fibrosis transmembrane conductance regulator, aspartylglucsaminidase, α-galactosidase A, cysteine transporter, acid ceremidase, acid α-L-fucosidase, protective protein, cathepsin A, acid β-glucosidase, acid β-galactosidase, iduronate 2-sulfatase, α-L-iduronidase, galactocerebrosidase, acid α-mannosidase, acid β-mannosidase, arylsulfatase B, arylsulfatase A, N-acetylgalactosamine-6-sulfate sulfatase, acid β-galactosidase, N-acetylglucosamine-1-phosphotransferase, acid sphingmyelinase, NPC-1, acid α-glucosidase, β-hexosamine B, heparan N-sulfatase, α-N-acetylglucosaminidase, α-glucosaminide N-acetyltransferase, N-acetylglucosamine-6-sulfate sulfatase, α-N-acetylgalactosaminidase, α-neuramidase, α-glucuronidase, β-hexosamine A and acid lipase, polyglutamine, α-synuclein, Ab peptide, tau protein and transthyretin.

### Pharmaceutical Compositions

A proteostasis regulator composition, useful in the present compositions and methods can be administered to a human patient per se, in the form of a stereoisomer, prodrug, pharmaceutically acceptable salt, hydrate, solvate, acid salt hydrate, N-oxide or isomorphic crystalline form thereof, or in the form of a pharmaceutical composition where the compound is mixed with suitable carriers or excipient(s) in a therapeutically effective amount, for example, to treat a proteostasis loss of function disorder, e.g., a lysosomal storage disease, or a gain of function disorder.

“Therapeutically effective amount” refers to that amount of the therapeutic agent, the proteostasis regulator composition, sufficient to result in the amelioration of one or more symptoms of a disorder, or prevent advancement of a disorder, cause regression of the disorder, or to enhance or improve the therapeutic effect(s) of another therapeutic agent. With respect to the treatment of a loss of function disorder, e.g., a lysosomal storage disease, or a gain of function disorder, a therapeutically effective amount refers to the amount of a therapeutic agent sufficient to reduce or eliminate the disease. Preferably, a therapeutically effective amount of a therapeutic agent reduces or eliminates the disease, by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. “Therapeutic protocol” refers to a regimen for dosing and timing the administration of one or more therapeutic agents, such as a small chemical molecule composition acting as a proteostasis regulator.

Pharmaceutically acceptable carriers are determined in part by the particular composition being administered, as well as by the particular method used to administer the composition. Accordingly, there is a wide variety of suitable formulations of pharmaceutical compositions for administering the antibody compositions (see, e.g., latest edition of Remington's Pharmaceutical Sciences, Mack Publishing Co., Easton, Pa., incorporated herein by reference). The pharmaceutical compositions generally comprise a proteostasis regulator composition in a form suitable for administration to a patient. The pharmaceutical compositions are generally formulated as sterile, substantially isotonic and in full compliance with all Good Manufacturing Practice (GMP) regulations of the U.S. Food and Drug Administration.

### Treatment Regimes

Aspects of the invention provide pharmaceutical compositions comprising one or a combination of proteostasis regulator compositions formulated together with a pharmaceutically acceptable carrier. Some compositions include a combination of multiple (e.g., two or more) proteostasis regulator compositions or derivative thereof.

In prophylactic applications, pharmaceutical compositions or medicaments are administered to a patient susceptible to, or otherwise at risk of a disease or condition, i.e., a proteostasis loss of function disorder or gain of function disorder, in an amount effective to eliminate or reduce the risk, lessen the severity, or delay the outset of the disease, including biochemical, histologic and/or behavioral symptoms of the disease, its complications and intermediate pathological phenotypes presenting during development of the disease. In therapeutic applications, compositions or medicaments are administered to a patient suspected of, or already suffering from such a disease in an amount effective to cure, or at least partially arrest, the symptoms of the disease (biochemical, histologic and/or behavioral), including its complications and intermediate pathological phenotypes in development of the disease. An amount adequate to accomplish therapeutic or prophylactic treatment is defined as a therapeutically- or prophalactically-effective dose. In both prophylactic and therapeutic regimes, agents are usually administered in several dosages until a sufficient immune response has been achieved. Typically, the immune response is monitored and repeated dosages are given if the immune response starts to wane.

### Effective Dosages

Effective doses of the proteostasis regulator composition, for the treatment of a proteostasis loss of function disorder or gain of function disorder, as described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

For administration of one or more proteostasis regulator compositions, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months. In some methods, two or more proteostasis regulator polypeptides, or mimetic, analog or derivative thereof, with different binding specificities are administered simultaneously, in which case the dosage of each proteostasis regulator composition is usually administered on multiple occasions. Intervals between single dosages can be a few days, weekly, monthly or yearly. Intervals can also be irregular as indicated by measuring blood levels of the proteostasis regulator composition or the proteostasis network composition in the patient. In some methods, dosage is adjusted to achieve an concentration of 1-1000 μg/ml of proteostasis regulator composition and in some methods 25-300 μg/ml. Alternatively, the proteostasis regulator compositions can be administered as a sustained release formulation, in which case less frequent administration is required. Dosage and frequency vary depending on the half-life of the compound in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of a proteostasis loss of function disorder or gain of function disorder. Thereafter, the patent can be administered a prophylactic regime.

Doses for a nucleic acid vector encoding a proteostasis regulator composition, range from about 10 ng to 1 g, 100 ng to 100 mg, 1 μg to 10 mg, or 30-300 μg DNA per patient. Doses for infectious viral vectors vary from 10-100, or more, virions per dose.

### Prodrugs

The present invention is also related to prodrugs of the agents obtained by the methods disclosed herein. Prodrugs are agents which are converted in vivo to active forms. R. B. Silverman, The Organic Chemistry of Drug Design and Drug Action, Academic Press, Chp. 8, 1992. Prodrugs can be used to alter the biodistribution (e.g., to allow agents which would not typically enter the reactive site of the protease) or the pharmacokinetics for a particular agent. For example, a carboxylic acid group, can be esterified, e.g., with a methyl group or an ethyl group to yield an ester. When the ester is administered to a subject, the ester is cleaved, enzymatically or non-enzymatically, reductively, oxidatively, or hydrolytically, to reveal the anionic group. An anionic group can be esterified with moieties (e.g., acyloxymethyl esters) which are cleaved to reveal an intermediate agent which subsequently decomposes to yield the active agent. The prodrug moieties may be metabolized in vivo by esterases or by other mechanisms to carboxylic acids.

Examples of prodrugs and their uses are well known in the art. e.g., Berge et al., J. Pharm. Sci. 66: 1-19, 1977. The prodrugs can be prepared in situ during the final isolation and purification of the agents, or by separately reacting the purified agent in its free acid form with a suitable derivatizing agent. Carboxylic acids can be converted into esters via treatment with an alcohol in the presence of a catalyst.

Examples of cleavable carboxylic acid prodrug moieties include substituted and unsubstituted, branched or unbranched lower alkyl ester moieties, (e.g., ethyl esters, propyl esters, butyl esters, pentyl esters, cyclopentyl esters, hexyl esters, cyclohexyl esters), lower alkenyl esters, dilower alkyl-amino lower-alkyl esters (e.g., dimethylaminoethyl ester), acylamino lower alkyl esters, acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, dilower alkyl amides, and hydroxy amides.

### Routes of Administration

A proteostasis regulator compositions for treatment or amelioration of a loss of function disorder or gain of function disorder can be administered by parenteral, topical, intravenous, oral, subcutaneous, intraarterial, intracranial, intraperitoneal, intranasal or intramuscular means for prophylactic as inhalants for proteostasis regulator compositions targeting a loss of function disorder, e.g., a lysosomal storage disease, or a gain of function disorder and/or therapeutic treatment. The most typical route of administration of an immunogenic agent is subcutaneous although other routes can be equally effective. The next most common route is intramuscular injection. This type of injection is most typically performed in the arm or leg muscles. Intramuscular injection or intravenous infusion are preferred for administration of antibody. In some methods, antibodies are administered as a sustained release composition or device, such as a Medipad™ device.

Agents of the invention can optionally be administered in combination with other agents that are at least partly effective in treating a condition characterized by dysfunction in protein homeostasis in a patient in need thereof.

### Formulation

A proteostasis regulator composition for the treatment of a loss of function disorder, e.g., a lysosomal storage disease, or a gain of function disorder are often administered as pharmaceutical compositions comprising an active therapeutic agent, i.e., and a variety of other pharmaceutically acceptable components. See latest edition of Remington's Pharmaceutical Science (Mack Publishing Company, Easton, Pa.). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, compositions of aspects of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking or expression as a fusion protein.

Alternatively, transdermal delivery can be achieved using a skin patch or using transferosomes. Paul et al., Eur. J. Immunol. 25: 3521-24, 1995; Cevc et al., Biochem. Biophys. Acta 1368: 201-15, 1998.

The pharmaceutical compositions are generally formulated as sterile, substantially isotonic and in full compliance with all Good Manufacturing Practice (GMP) regulations of the U.S. Food and Drug Administration.

### Toxicity

Preferably, a therapeutically effective dose of proteostasis regulator compositions, described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. e.g., Fingl et al., The Pharmacological Basis of Therapeutics, Ch. 1., 1975.

### Kits

Also within the scope of the invention are kits comprising a proteostasis regulator composition of aspects of the invention and instructions for use. The kit can further contain a least one additional reagent, or one or more additional human antibodies of aspects of the invention (e.g., a human antibody having a complementary activity which binds to an epitope in the antigen distinct from the first human antibody). Kits typically include a label indicating the intended use of the contents of the kit. The term label includes any writing, or recorded material supplied on or with the kit, or which otherwise accompanies the kit.

Other embodiments and uses will be apparent to one skilled in the art in light of the present disclosures.

The invention will be further described with reference to the following examples; however, it is to be understood that the invention is not limited to such examples.

### Exemplary Embodiments

### Example 1

### Celastrol is a Proteostasis Regulator in Gaucher Disease Patient-Derived Fibroblasts

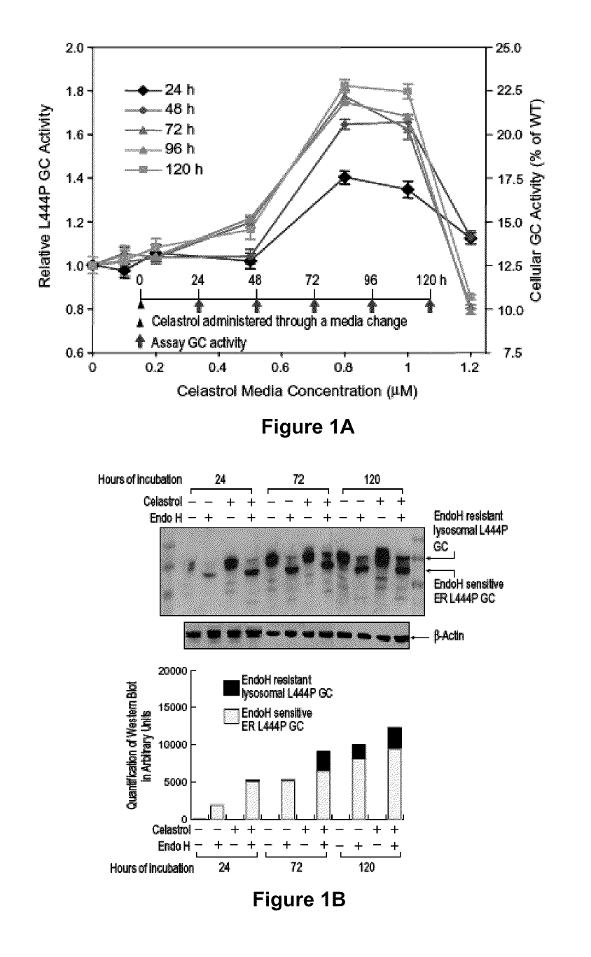

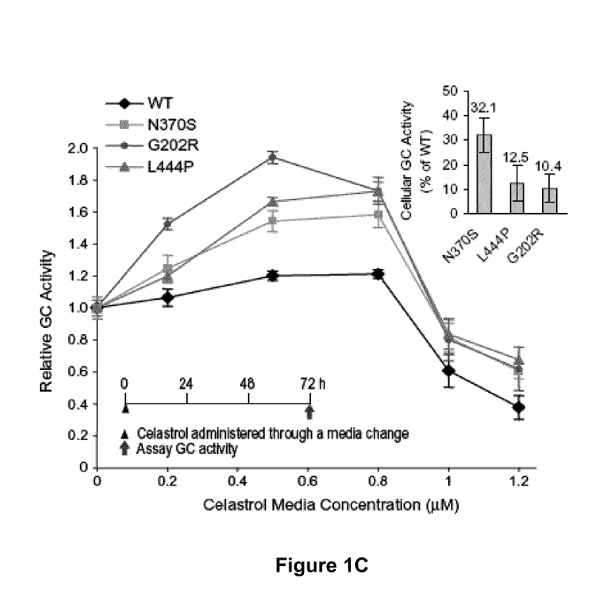

We administered small molecules known to influence proteostasis, including salubrinal [Boyce et al., Science 307: 935-939, 2005], celastrol [Westerheide et al., J Biol Chem 279: 56053-56060, 2004], indomethacin, and natriumsalycilate, to a L444P GC Gaucher fibroblast cell line (GM08760) known to be resistant to pharmacologic chaperoning [Sawkar et al., Chem Biol 12: 1235-1244, 2005; Sawkar et al., Proc Natl Acad Sci USA 99:15428-15433, 2002; Sawkar et al., ACS Chem Biol 1: 235-251, 2006b]. Lysosomal GC activity was evaluated using the previously reported intact fibroblast assay with the synthetic substrate 4-methylumbellifery β-D-glucoside [Sawkar et al., Proc Natl Acad Sci USA 99:15428-15433, 2002]. We also demonstrated that a natural substrate, C12 β-D-glucosyl ceramide, could be broken down by a variety of cell lines harboring wild type (WT) and variant GC employing a lysed cell assay, wherein the reaction was followed by thin layer chromatography. Since enzyme activity is highly dependent on the assay conditions utilized, mutant lysosomal enzyme activities are reported as a fold-change relative to mutant GC activity in untreated cells and as the fraction of WT GC activity measured identically (See the inset to FIG. 1C for the relative lysosomal activities of the Gaucher disease associated GC variants; the lowered activities are a consequence of lowered specific activities and lowered lysosomal concentrations). Sawkar et al., Chem Biol 12: 1235-1244, 2005.

Celastrol (0.8 μM), but not the other compounds evaluated, increased the activity of L444P GC 1.8-fold (to 23% of cellular WT GC activity) after a 72 h incubation period at 37° C. (FIG. 1A). This is notable because we had never observed a statistically significant increase in L444P GC activity previously with pharmacologic chaperones. Sawkar et al., Chem Biol 12: 1235-1244, 2005; Sawkar et al., Proc Natl Acad Sci USA 99:15428-15433, 2002; Sawkar et al., ACS Chem Biol 1: 235-251, 2006b. Celastrol is known to be a heat shock factor 1 (HSF 1) transcriptional activator that induces the heat shock response in human cells, a conserved reaction of the cytoplasm to protein denaturation/aggregation enabled by the up-regulation of molecular chaperones and other macromolecules to reestablish proteostasis upon stress abatement. Westerheide et al., J Biol Chem 279: 56053-56060, 2004; Lindquist, Ann Rev Biochem 55: 1151-1191, 1986; Westerheide et al., J Biol Chem 280: 33097-33100, 2005. Celastrol's narrow therapeutic window of 0.5 to ≈1 μM, resulting from cytotoxicity at higher concentrations according to trypan blue staining, would be a concern if celastrol itself were being considered as a drug candidate. Instead it is being used here to demonstrate proof of principle and to motivate the discovery of less toxic equivalents.

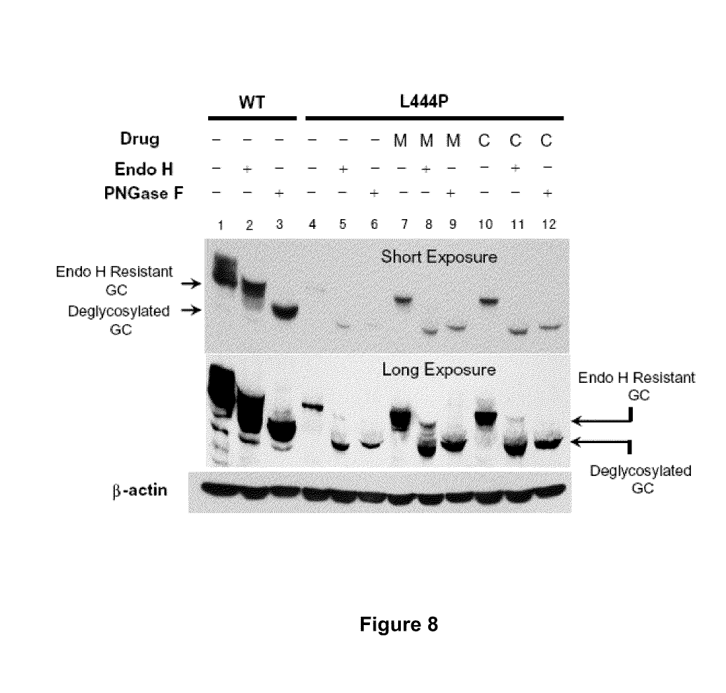

The partial restoration of L444P GC proteostasis was further supported by analysis of the distinct glycosylation pattern associated with GC trafficking through the Golgi compartment. Ron et al., Hum Mol Genet. 14: 2387-2398, 2005; Zimmer et al., J Pathol 188: 407-414, 1999. Fibroblasts grown in the presence or absence of celastrol were lysed at the indicated times and the glycosylation of L444P GC was analyzed by Western blot after treatment with endoglycosidase H (endo H) (FIG. 1B). Digestion with PNGase F confirms that the high MW endo H resistant band was glycosylated (FIG. 8). A low molecular weight band corresponding to the endo H-sensitive, partially glycosylated GC that has not left the ER is typically detected after endo H treatment. Ron et al., Hum Mol Genet. 14: 2387-2398, 2005; Sawkar et al., ACS Chem Biol 1: 235-251, 2006b; Schmitz et al., Int J Biochem Cell Biol 37: 2310-2320, 2005; Zimmer et al., J Pathol 188: 407-414, 1999. A high molecular weight band which corresponds to the endo H-resistant lysosomal GC glycoform is observed for WT fibroblasts (FIG. 8 lane 2), but only faintly, if at all, for the Gaucher disease-associated GC variants. Ron et al., Hum Mol Genet. 14: 2387-2398, 2005; Sawkar et al., ACS Chem Biol 1: 235-251, 2006b; Schmitz et al., Int J Biochem Cell Biol 37: 2310-2320, 2005; Zimmer et al., J Pathol 188: 407-414, 1999. Densitometry quantification of the post-ER GC glycoform band reveals that it was more intense in cells treated with celastrol (cf. black bars, FIG. 1B). The L444P GC in celastrol treated fibroblasts is a mixture of enzymatically active, natively folded, natively glycosylated GC (black bars, FIG. 1B) and ER retained GC that is not properly glycosylated (white bars, FIG. 1B).

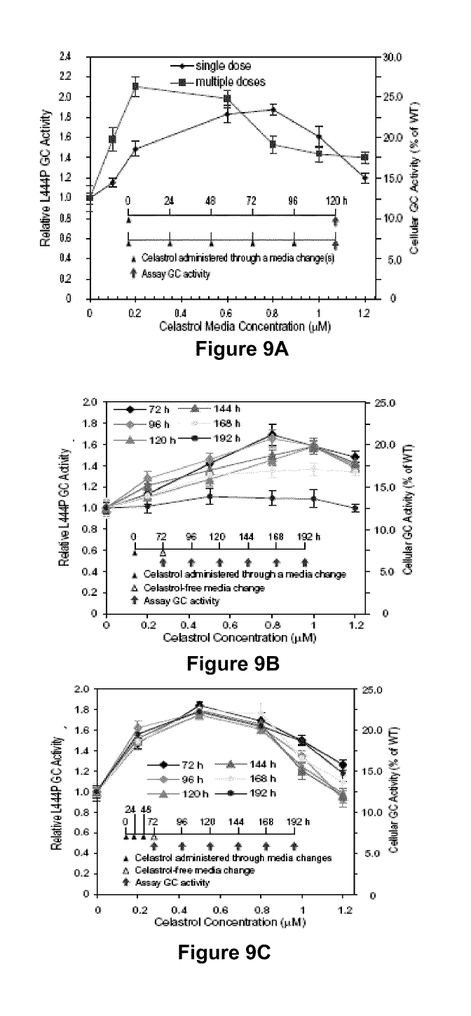

Celastrol treatment (<0.8 μM media concentration, 72 h) of Gaucher patient-derived fibroblasts harboring N370S and G202R GC mutations, two variants retained in the ER, revealed a 1.5-fold increase (to ≈39% of cellular WT GC activity) and a 1.9-fold increase (to ≈20% of cellular WT GC activity), respectively, (FIG. 1C). It is notable that the activity of L444P GC, thought to be a severe neuropathic mutation, is restored by celastrol to the same extent as the activity of N370S GC. Sawkar et al., Proc Natl Acad Sci U S A 99:15428-15433, 2002. L444P GC fibroblasts exposed repeatedly to variable concentrations of celastrol at t=0, 24, 48, 72, and 96 h exhibited a 2.1-fold increase in activity (to ≈26% of WT GC activity) at t=120 h (0.2 μM Celastrol) (FIG. 9A, red line), a slight increase over a single celastrol exposure (FIG. 9A, blue line). Investigating the temporal dependence of the L444P GC activity increase revealed increased activity for 96 h after a single dose and for 120 h with multiple doses (see FIGS. 9B and 9C). Thus, it is apparent that mutant GC is sensitive to its proteostasis environment.

FIG. 1 shows Celastrol treatment enhances activity of variant glucocerebrosidases (GCs) and their cellular trafficking to the lysosome. A) Relative lysosomal GC activity of L444P GC fibroblasts in celastrol (0.2 to 1.2 μM) containing culture media. Celastrol was added at t=0 and GC activities were assayed every 24 h for 120 h without a media change. Reported activities were normalized to the activity of untreated cells of the same type (left y axis) and expressed as the percentage of WT GC activity (right y axis). B) Western blot analysis of L444P GC trafficking within fibroblasts after 24, 72, and 120 h exposure to 0.8 μM celastrol. GC bands were detected with mouse anti-GC antibody and β-actin serves as a gel loading control. The western blot bands in the endoH treated samples were quantified by Java Image processing and analysis software from the NIH (http://rsb.info.nih.gov/ij/) The white portion of the bars represents quantification of the lower, endoH sensitive bands and the black portion of the bars represents the higher MW, endoH resistant bands. C) Relative lysosomal activity of wild type GC and Gaucher disease-associated N370S, G202R, and L444P GC variants in patient-derived fibroblasts. Cells were grown and treated with celastrol as in FIG. 1A, and the normalized GC activity was evaluated after a 72 h incubation period. The inset displays the GC variant enzyme activity expressed as the percentage of WT GC activity under our assay conditions, as reported previously. Sawkar et al., Chem Biol 12: 1235-1244, 2005.

### Example 2

### The Proteasome Inhibitor MG-132 is a Proteostasis Regulator in L444P GC Fibroblasts

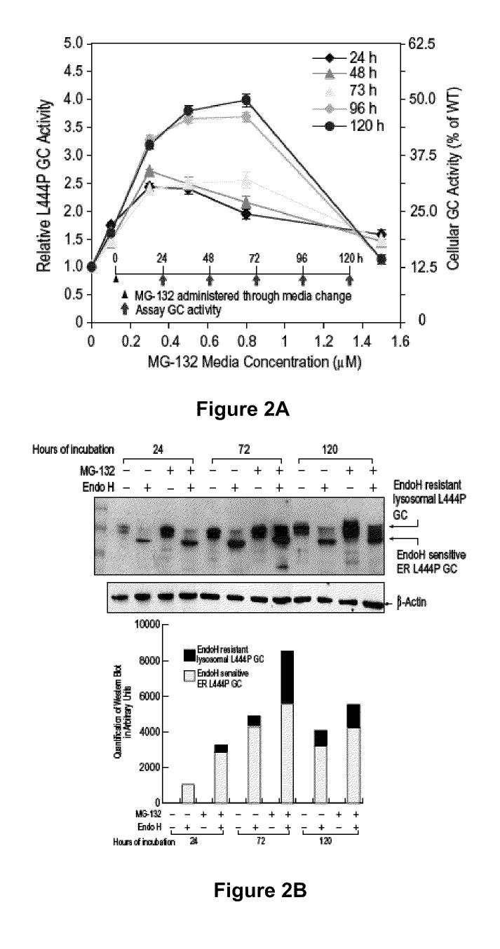

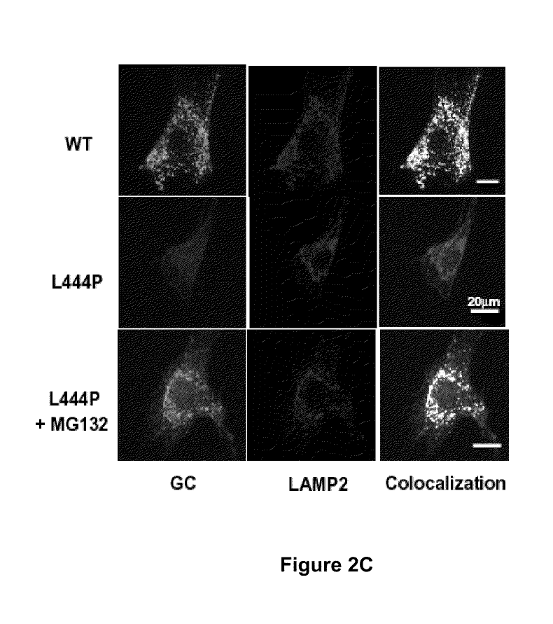

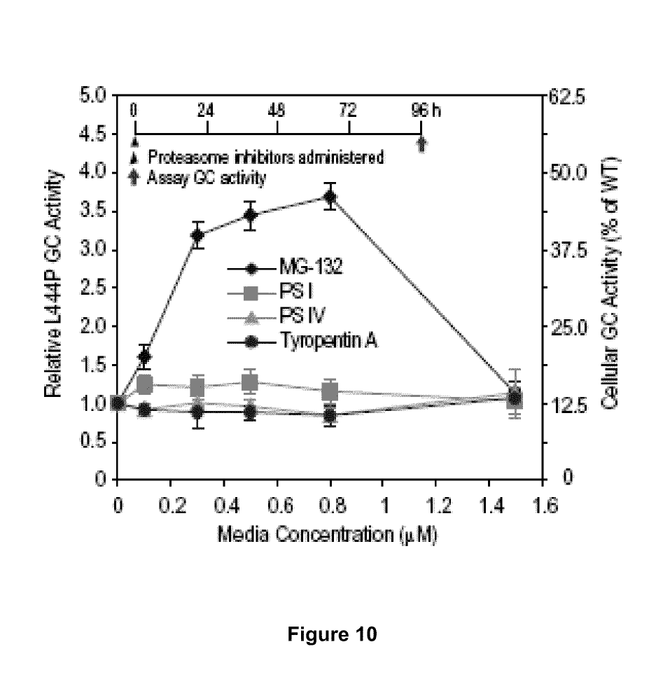

Because proteasome inhibitors both enhance chaperone expression levels and inhibit ERAD, suggested to us that they could be potent proteostasis regulators. Bush et al., J Biol Chem 272: 9086-9092, 1997; Liao et al., J Cell Biochem 99: 1085-1095, 2006; Chillaron et al., Mol Biol Cell 11: 217-226, 2000; Wiseman et al., Cell 131: 809-821, 2007. To test this hypothesis, L444P GC fibroblasts were subjected to a single exposure of the known proteasome inhibitors MG-132, PSI, PS IV, and Tyropentin A, at media concentrations ranging from 0.1 to 1.5 μM. L444P GC activity was monitored every 24 h for 96 h. While PS IV and Tyropentin A did not enhance L444P GC activity (FIG. 10), PS I resulted in a modest 1.25-fold increase (FIG. 10), whereas MG-132 increased L444P GC activity 4-fold (to ≈50.0% of WT GC activity) after 120 h (FIG. 2A). Western blot analysis revealed a striking increase in the endo H-resistant GC band in MG-132 treated cells, consistent with an increase in the mature, folded, lysosomal form of L444P GC, especially at 72 h (FIG. 2B, black bars). The notable increase in the intensity of the endo H-sensitive ER band of L4444P GC is consistent with MG-132 serving as an ERAD inhibitor for the first 72 h (FIG. 2B, white bars) and is larger than the increase observed in this band with celastrol administration (FIG. 1B). Optimization of MG-132 dosing regimen (e.g. multiple doses spaced more than 72 h apart) could lead to further enhancements in L444P GC lysosomal activity. While general proteasome inhibition is not sufficient for GC proteostasis regulator function, MG-132 appears to be a proteostasis regulator.

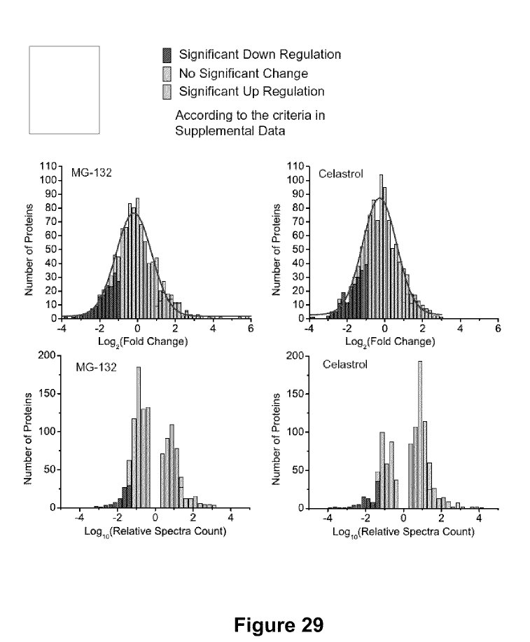

Mass spectrometry-based proteomic analysis (multidimensional protein identification technology [MudPIT]) was used to understand the influence of PR treatment on global protein biogenesis (Liu et al., Anal. Chem. 76, 4193-4201 (2004); Liao et al., J. Proteome Res. 6, 1059-1071 (2007); Rikova et al., Cell 131, 1190-1203 (2007)). Treatment of L444P GC fibroblasts with MG-132 (0.8 μM) for 3 days upregulated 198 proteins and downregulated 255 proteins (FIG. 29, left), while treatment with celastrol (0.8 μM) for 3 days upregulated 199 proteins and downregulated 292 proteins among the 2100 proteins detected in the untreated and treated samples (FIG. 29, right). Thus, PRs can provide a corrective environment for energetically destabilized enzymes while having only modest effects on the proteome.

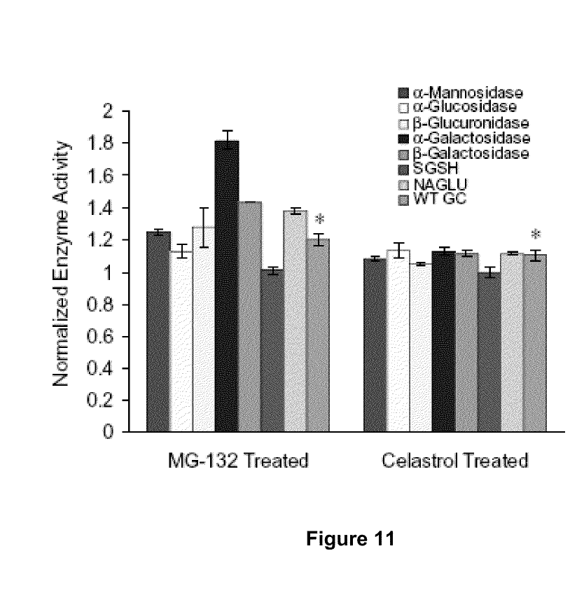

Immunofluorescence studies reveal that WT GC colocalized with the lysosomal marker LAMP2 (FIG. 2C, top row, GC in green, LAMP2 in red, with the overlap artificially colored white) verifying the proper trafficking of WT GC to the lysosome. Sawkar et al., ACS Chem Biol 1: 235-251, 2006b. L444P GC fibroblasts were incubated without and with 0.25 μM MG-132 for 3 d prior to plating for microscopy. L444P GC was barely visible without drug treatment, due to extensive ERAD. L444P GC was easily detected after MG-132 treatment (FIG. 2C, cf. rows 3 and 2) and exhibited colocalization with the lysosomal marker LAMP2 (FIG. 2C, row 3, column 3). Collectively, the activity, the endo H resistance, and fluorescence microscopy data (FIG. 2) demonstrate that properly folded L444P GC exited the ER, trafficked through the Golgi and reached the lysosome. As a control, the influence of MG-132 and celastrol on the cellular activity of 7 WT lysosomal hydrolases (FIG. 11) was evaluated in L444P GC fibroblasts. Celastrol treatment did not increase their enzymatic activity significantly. MG-132 increased the activity of α-galactosidase 1.8-fold, whereas the activity of other enzymes monitored increased an average of 1.2-fold (FIG. 11). Neither was WT GC activity in normal fibroblasts increased with celastrol treatment.

FIG. 2 shows the proteasome inhibitor MG-132 potently enhances GC activity and promotes its cellular trafficking to the lysosome within L444P GC fibroblasts. A) GC activity of L444P GC fibroblasts exposed to MG-132 at t=0 and incubated without a media change for 120 h. GC activities were measured at 24, 48, 72, 96 and 120 h, and reported relative to the activity of untreated cells of the same type (left y axis) and as the percentage of WT GC activity (right y axis). B) Western blot analysis of L444P GC from a fibroblast cell line exposed to MG-132 (0.8 μM) at t=0. Cellular protein was harvested at 24, 72 and 120 h and the ER and lysosomal GC glycoforms were measured and quantified as described in FIG. 1B. C) Immunofluorescence microscopy analysis of GC in L444P GC and WT cells (positive control). L444P GC cells were incubated with 0.25 μM MG-132 for 3 days (bottom row) or untreated (middle row). GC was detected using the mouse anti-GC antibody 8E4 (column 1); rabbit anti-LAMP2 antibody was used as a lysosomal marker (column 2). Colocalization of GC (green) and LAMP2 (red) was artificially colored white (column 3).

FIG. 8 shows Western blot analysis of GC trafficking in L444P GC fibroblasts. L444P GC fibroblasts were treated with 0.25 μM MG-132 for 72 h (Marked as M), or 0.8 μM celastrol for 72 h (Marked as C). Untreated WT and L444P cells served as positive and negative controls, respectively. Equal amount of total proteins from lysed cells were digested with buffer only, EndoH, or PNGase F before separation in a 10% SDS-PAGE gel and detection using mouse anti-GC antibody 2E2. EndoH resistant GC bands reflect the mature lysosomally localized glycoform of GC. PNGase F digestion yielded the deglycosylated GC form. Both the gel images were taken from the same blot with different exposure times. Longer exposure is required to visualize the EndoH resistant bands. α-actin serves as a loading control.

FIG. 9 shows optimization of celastrol dosing regime in L444P GC fibroblasts. Reported activities were normalized to the activity of untreated L444P cells (left y axis) and expressed as the percentage of WT GC activity (right y axis). A) L444P GC activity within fibroblasts treated with celastrol for 120 h at medium concentrations ranging from 0.1 to 1.2 μM is reported relative to the activity of untreated cells. The blue curve indicates administration of celastrol at t=0, with no media or celastrol changes thereafter, while the red curve results from celastrol administration at t=0, 24, 48, 72, and 96 h, enabled by media changes. B) Relative GC activity of L444P GC fibroblasts exposed to celastrol at time 0, thereafter the media was replaced at 72 h with celastrol free media, and L444P GC activity was measured at 72, 96, 120, 144, 168 and 192 h. The celastrol-mediated L444P GC activity gains increased activity for 96 h after a single dose. C) Relative GC activity of L444P GC fibroblasts exposed to celastrol at t=0, 24 and 48 h and the media was replaced by celastrol free media at 72 h and L444P GC activity was measured at 72, 96, 120, 144, 168 and 192 h. This exhibited retention of activity for 120 h. 7

FIG. 10 shows the effect of proteasome inhibitors on GC activity in L444P GC fibroblasts. L444P GC fibroblasts were exposed to a variety of proteasome inhibitors (MG-132, PS I, PS IV, and Tyropentin A) at t=0 at medium concentrations ranging from 0.1 to 1.5 μM. The cells were incubated for 96 h without a media change, and the GC activities were measured, and reported relative to the activity of untreated cells of the same type (left y axis) and as the percentage of WT GC activity (right y axis).

FIG. 11 shows the effect of MG-132 and celastrol on the activity of other WT lysosomal enzymes in L444P fibroblasts, as well as GC in WT GC fibroblasts (indicated by asterisk). After incubation with 0.8 μM MG-132 or 0.8 μM celastrol for 24 h, L444P GC fibroblasts were assayed for the activities of α-mannosidase, α-glucosidase, α-glucuronidase, α-galactosidase, α-galactosidase, heparan sulfate sulfamidase (SGSH), and α-N-acetylglucosaminidase (NAGLU), and WT GC fibroblasts were assayed for the GC activity, with their corresponding substrates using a lysed cell enzyme activity assay, as previously described. Sawkar et al. Chem Biol 12: 1235-1244, 2005. The enzyme activity of treated cells was normalized against that of untreated cells of the same type. Each data point reported was evaluated at least in triplicate in each plate, and on three different days.

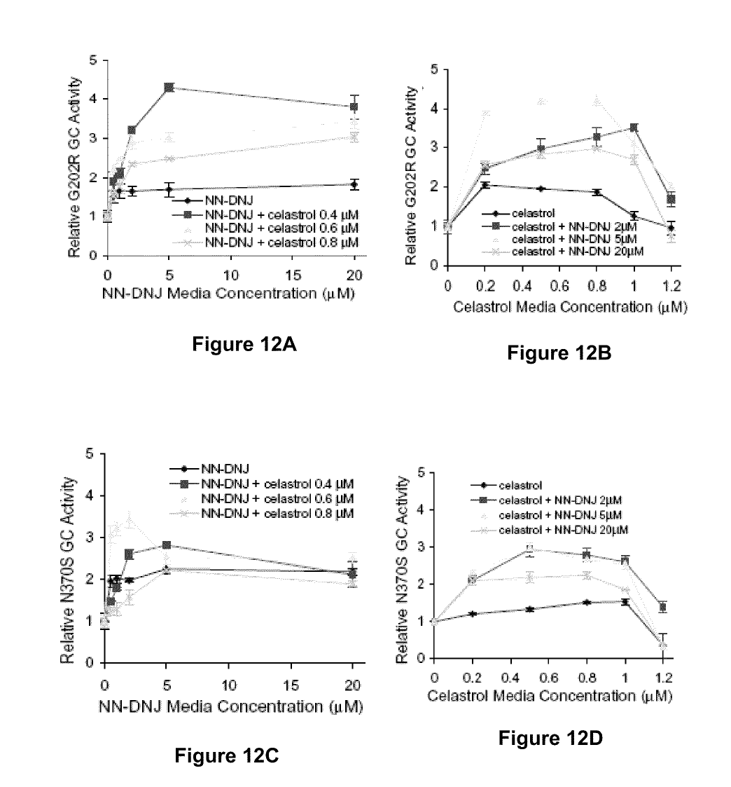

FIG. 12 shows 2D plots showing GC activity of G202R and N370S GC patient derived fibroblasts cultured with media containing celastrol and NN-DNJ. A) Relative GC activity of G202R GC fibroblast cell lines. Four sets of cultures were prepared and incubated with celastrol at 0, 0.4, 0.6 or 0.8 μM. Each set was additionally supplemented with NN-DNJ at medium concentration ranging from 1 to 20 μM. GC activities were measured after 4 days of growth and normalized by the GC activity of untreated cells. B) Relative GC activity of G202R GC fibroblast cell lines. Four sets of cultures were prepared and incubated with NN-DNJ at 0, 2, 5 or 20 μM. Each set was additionally supplemented with celastrol at medium 8 concentration ranging from 0.2 to 1.2 μM. GC activities were measured after 4 days of growth and normalized by the GC activity of untreated cells. C) Relative GC activity of N370S GC fibroblast cell lines. Cells were grown and treated, and GC activities measured as described in A. D) Relative GC activity of N370S GC fibroblast cell lines. Cells were grown and treated, and GC activities measured as described in B.

### Example 3

### Pharmacologic Chaperones and Proteostasis Regulators Exhibit Synergy

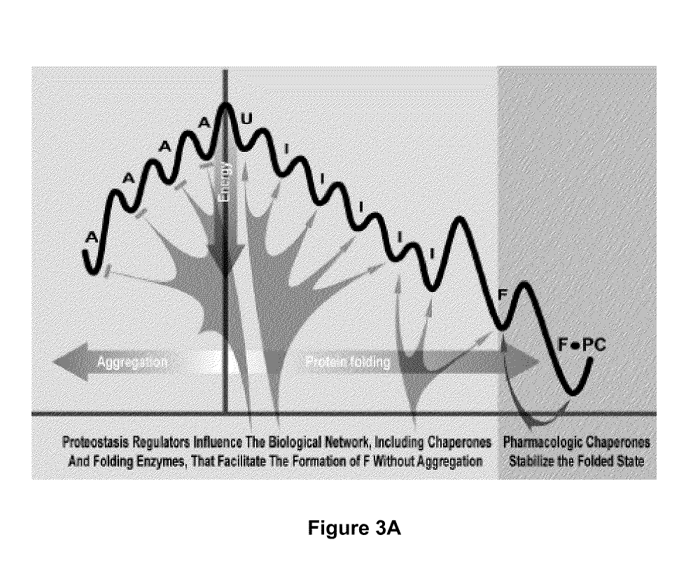

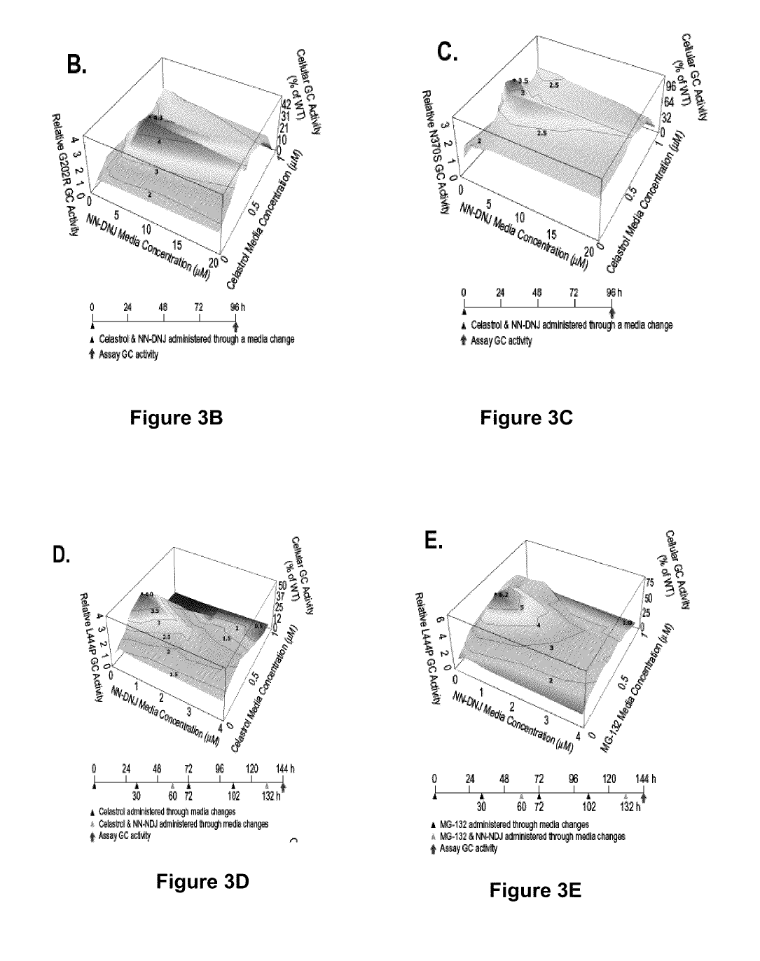

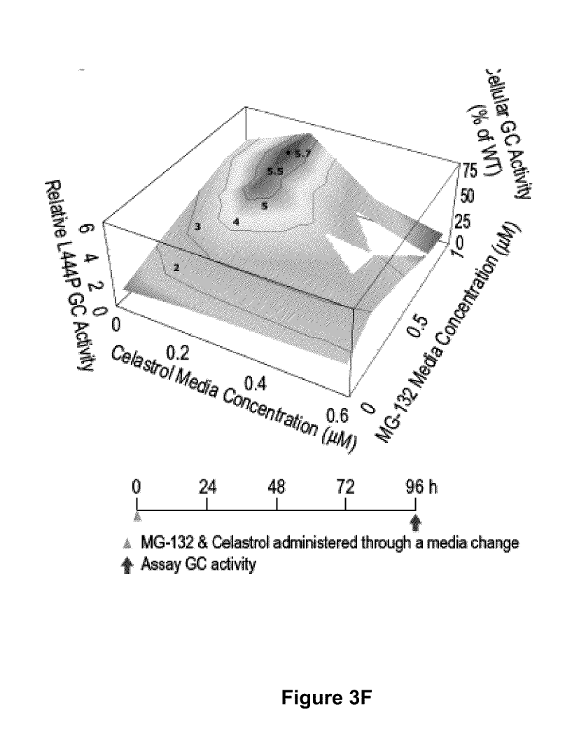

Addition of sub-inhibitory concentrations of GC inhibitors/pharmacologic chaperones, such as N-(n-nonyl)deoxynojirimycin (NN-DNJ; <30 μM), to N370S and G202R GC Gaucher disease patient-derived fibroblasts increased mutant GC folding, trafficking efficiency and activity. Sawkar et al., Chem Biol 12: 1235-1244, 2005; Sawkar et al., Proc Natl Acad Sci USA 99:15428-15433, 2002; Sawkar et al., ACS Chem Biol 1: 235-251, 2006b; Yu et al., J Med Chem 50: 94-100, 2007b. We therefore wondered whether combining a pharmacologic chaperone with a proteostasis regulator, such as celastrol, could have a synergistic effect on enhancing GC proteostasis, owing to their distinct mechanisms of action. Bouvier, Chem Biol 14: 241-242, 2007; Fan et al., Nat Med 5: 112-115, 1999; Sawkar et al., Proc Natl Acad Sci USA 99:15428-15433, 2002. GC pharmacologic chaperones stabilize the folded state ensemble, enabling a higher population of GC to engage the ER export machinery (FIG. 3A), whereas a proteostasis regulator upregulates the chaperone mediated folding pathways, enhancing GC folding efficiency by chaperone binding to partially folded intermediates to facilitate their folding while reducing aggregation, FIG. 3A. Wiseman et al., Cell 131: 809-821, 2007.

NN-DNJ and celastrol were co-administered to fibroblasts harboring GC mutations known to be amenable to pharmacologic chaperoning (N370S and G202R GC), as a function of concentration. G202R fibroblasts incubated with celastrol alone (0.4 μM) exhibited a 2-fold increase or a 100 unit or 100% increase in activity, while a 1.8-fold or 80 unit increase in G202R GC activity was observed with NN-DNJ (≦20 μM) alone. Co-administration of celastrol (0.4 μM) and NN-DNJ (5 μM) resulted in a 4.2-fold or 320 unit increase in G202R activity (to ≈44% of WT GC activity) (FIGS. 3B and 12A and 12B), nearly double the 2.8-fold or 180 unit sum, demonstrating a synergistic effect. A strictly analogous experiment was performed using N370S GC fibroblasts: celastrol alone (0.8 μM) resulted in a 1.5-fold increase in GC activity, whereas NN-DNJ alone (<20 μM) resulted in a 2.2-fold increase. N370S GC fibroblasts treated with 0.5 μM celastrol and 2 μM NN-DNJ exhibited a 3.5-fold increase in N370S GC activity (to ≈112% of WT GC activity), which is greater than the 2.7-fold sum, again demonstrating a synergistic effect (FIGS. 3C and 12C and D). It is likely that dosing regimen optimization would yield further synergistic increases in G202R and N370S GC folding, trafficking, and function.

### Example 4

### Proteostasis Regulator and Pharmacologic Chaperone Synergy in the Refractory L444P GC Cell Line

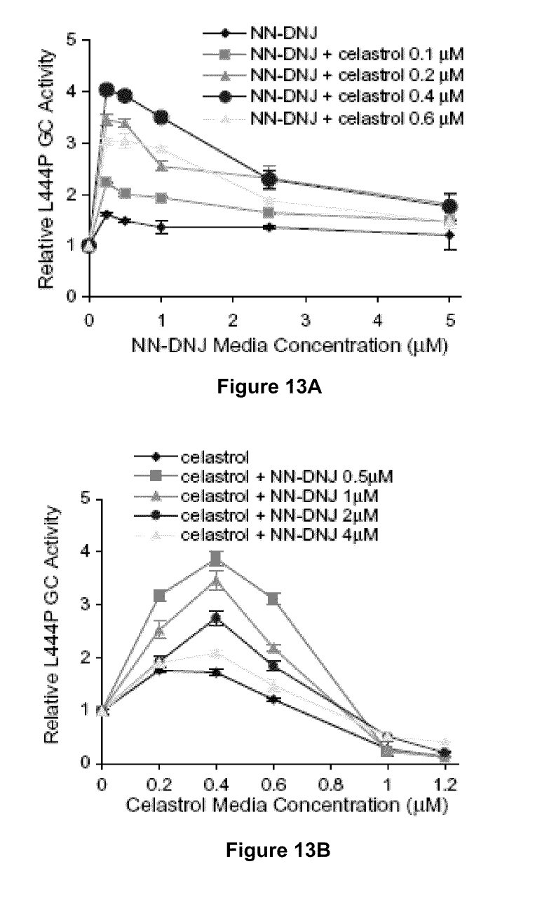

Although L444P GC is usually not amenable to pharmacologic chaperoning under conditions where N370S and G202R GC are, we tried a similar set of experiments with L444P GC by coadministering NN-DNJ and celastrol. Sawkar et al., Chem Biol 12: 1235-1244, 2005; Sawkar et al, Proc Natl Acad Sci USA 99:15428-15433, 2002. A barely significant 1.2-fold increase in L444P GC activity is achievable by incubating the cells for up to 12 h with NN-DNJ alone at concentrations ≦2 μM (FIG. 13A). When celastrol and NN-DNJ were combined at the optimal single dosing concentrations for enhancing N370S and G202R GC activity in fibroblasts, the observed L444P GC activity was lower than that obtained with celastrol alone (1.8-fold enhancement, FIG. 1A). Moreover, further experiments revealed that the decrease in GC activity was proportional to the NN-DNJ concentration used, suggesting that L444P GC is very sensitive to GC inhibition by NN-DNJ. Sawkar et al., ACS Chem Biol 1: 235-251, 2006b. To circumvent this sensitivity, brief pulses (12 h) of NN-DNJ, envisioned to keep the cellular NN-DNJ concentration below that where inhibition of lysosomal L444P GC would dominate over pharmacologic chaperoning, were used. This dosing schedule (FIG. 3D) resulted in a 3.9-fold (290 unit) increase in L444P GC activity at 144 h (to 49% of WT GC activity), which is nearly 300% greater than the 2.0-fold or 100 unit sum, demonstrating a synergistic effect (also see FIGS. 13A and 13B). Further optimization of the dosing regimen could be useful for neuropathic Gaucher disease intervention.

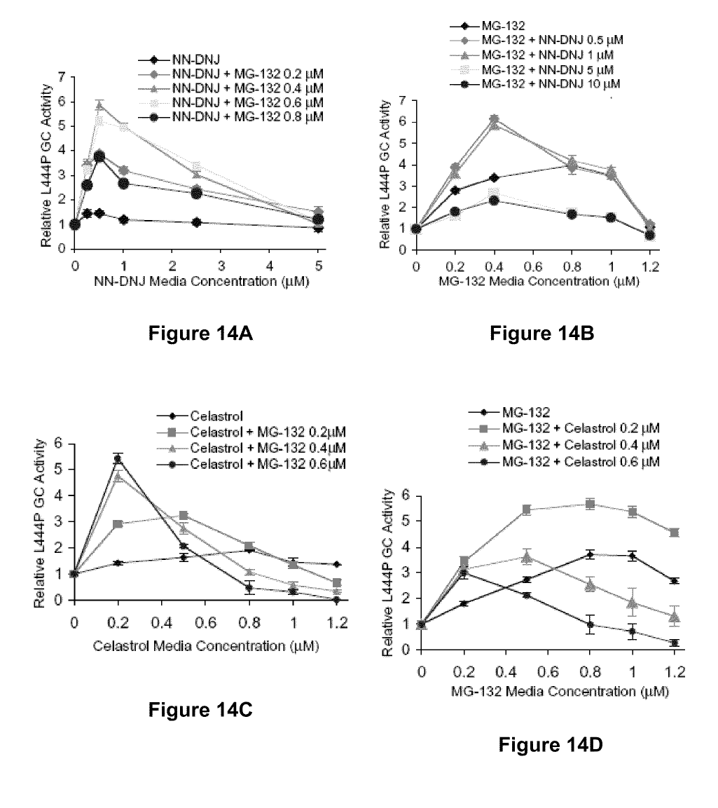

To probe the generality of pharmacologic chaperone and proteostasis regulator synergy, NN-DNJ and MG-132 were coadministered to L444P GC fibroblasts using the optimized dosing protocol established for the synergistic use of celastrol and NN-DNJ (FIG. 3D). A 6.2-fold increase in L444P GC activity (to ≈78% of cellular WT GC activity) was observed (MG-132 (0.4 μM) and NN-DNJ (0.5 μM)) (FIGS. 3E, and 14A and 14B).

### Example 5

### Co-Administration of Two Proteostasis Regulators Exhibits Synergy

Since proteostasis regulators and pharmacologic chaperone in combination exhibit a synergistic GC rescue, we wondered whether a combination of proteostasis regulators would afford synergy. Co-administration of MG-132 (0.6 μM) and celastrol (0.2 μM) to L444P GC fibroblasts resulted in a synergistic 6-fold (500 unit) increase in L444P GC activity (to ≈75.0% of cellular WT GC activity) after a 96 h incubation period (FIGS. 3F, 14C and 14D). L444P GC activity is enhanced 4-fold (300 units) by MG-132 alone, and 1.8-fold (80 units) by celastrol alone. These data demonstrate that the combined use of proteostasis regulators can be powerful.

FIG. 3 shows pharmacologic chaperones and proteostasis regulators exhibit synergy. A) Insights into distinct mechanisms of action of pharmacologic chaperones and proteostasis regulators. B-E) GC activities within patient-derived fibroblasts exposed to media containing celastrol and NN-DNJ, or MG-132 and NN-DNJ. In all the 3D plots, celastrol/MG132 and NN-DNJ media concentrations are shown on the x and y-axes, and the mutant GC activities on the z-axis. 2D plots of relative mutant GC activities vs. NN-DNJ and celastrol/MG132 concentrations are reported in FIGS. 12, 13, 14A and 14B. The dosing schematic is depicted at the bottom of FIGS. 3B-3E. Reported activities were normalized to the activity of untreated cells of the same type (left z axis) and expressed as the percentage of WT GC activity (right z axis). F) GC activity of L444P GC fibroblast cell lines exposed to celastrol and MG-132 at t=0. The 3D plot represents the celastrol and MG-132 media concentrations on the x and y-axes, and L444P GC activity on the z axis, measured after 96 h without a media change, relative to the activity of untreated cells of the same type (left z axis) and as the percentage of WT GC activity (right z axis). White areas reflect regions where the data are insufficient to interpolate. 2D plots of relative L444P GC activity vs. celastrol and MG-132 concentrations are reported in FIGS. 14C and 14D, respectively.

FIG. 13 shows cells were plated and treated according to the same experimental design described in FIG. 12 with the exception that the incubation medium was replaced at t=0, 30, 60, 72, 102, and 132 h. Media was supplemented with celastrol at t=0, 30, 72, and 102 h, while it was supplemented with both celastrol and NN-DNJ at t=60 and 132 h (see also the schematic on the bottom of FIG. 3D). L444P GC activity was measured after 144 h and normalized to the activity of untreated cells. A) Relative GC activity of L444P GC fibroblasts incubated with media concentration of NN-DNJ ranging from 0.25 to 5 μM and a constant concentration of celastrol of 0, 0.1, 0.2, 0.4, or 0.6 μM. B) Relative GC activity of L444P GC fibroblasts incubated with medium concentration of celastrol ranging from 0.2 to 1.2 μM and a constant concentration of NN-DNJ of 0, 0.5, 1, 2, or 4 μM.

FIG. 14 shows relative L444P GC activity in patient derived fibroblasts cultured with media containing MG-132 and celastrol, or MG-132 and NN-DNJ. Relative L444P GC activity was normalized to the activity of untreated cells of the same type. A) Relative GC activity of L444P GC fibroblasts incubated with medium concentrations of NN-DNJ ranging from 0.25 to 5 μM and a constant concentration of MG-132 of 0, 0.2, 0.4, 0.6, or 0.8 μM. The media was replaced at multiple times according to the same procedures described for celastrol and NN-DNJ in FIG. 13 and represented in the schematic of FIG. 3E, and the GC activity assay was performed after 6 days. B) Relative GC activity of L444P GC fibroblasts incubated with 9 medium concentrations of MG-132 ranging from 0.2 to 1.2 μM and a constant concentration of NN-DNJ of 0, 0.5, 1, 5, or 10 μM. The media was replaced according to the same procedures described for celastrol and NN-DNJ in FIG. 13 and represented in the schematic of FIG. 3E, and the GC activity assay was performed after 6 days. C) Relative GC activity of L444P GC fibroblasts incubated with medium concentrations of celastrol ranging from 0.2 to 1.2 μM and a constant concentration of MG-132 of 0, 0.2, 0.4, or 0.6 μM. L444P GC fibroblast cell lines were exposed to celastrol and MG-132 at t=0, and relative L444P GC activity was measured after 4 days of growth. D) Relative GC activity of L444P GC fibroblasts incubated with medium concentrations of MG-132 ranging from 0.2 to 1.2 μM and a constant concentration of celastrol of 0, 0.2, 0.4, or 0.6 μM. L444P GC fibroblast cell lines were exposed to celastrol and MG-132 at t=0, and relative L444P GC activity was measured after 4 days of growth.

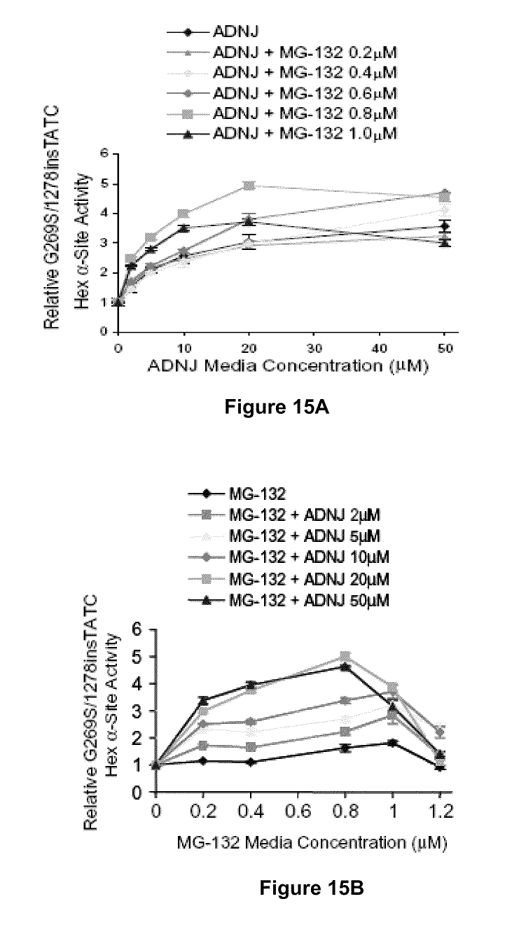

FIG. 15 shows relative Hex α-site activity in G269S/1278insTATC HexA Tay-Sachs fibroblast cell line cultured with media containing MG-132 and ADNJ. G269S/1278insTATC HexA fibroblast cell lines were exposed to MG-132 and ADNJ at t=0, and relative Hex á-site activity was measured after 6 days of growth, relative to the activity of untreated cells of the same type. A) Relative Hex α-site activity of G269S/1278insTATC HexA cells incubated with medium concentrations of ADNJ ranging from 2 to 50 μM and a constant concentration of MG-132 of 0, 0.2, 0.4, 0.6, 0.8 or 1.0 μM. B) Relative Hex αsite activity of G269S/1278insTATC HexA cells incubated with medium concentrations of MG-132 ranging from 0.2 to 1.2 μM and a constant concentration of ADNJ of 0, 2, 5, 10, 20 or 50 μM.

### Example 6

### Celastrol and MG-132 Also Serve as Proteostasis Regulators in Tay-Sachs Disease

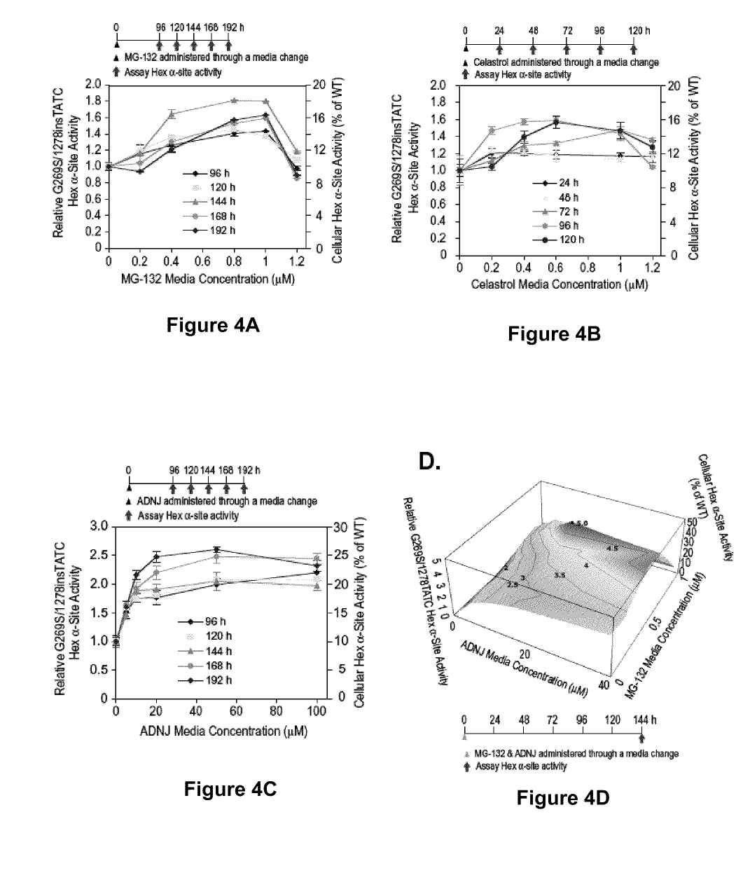

Celastrol and MG-132 should be able to restore proteostasis in other loss-of-function diseases associated with compromised mutant protein folding in the secretory pathway. We therefore evaluated the ability of these proteostasis regulators to restore partial function to HexA, a heterodimeric enzyme composed of α- and β-subunits that degrades GM2 gangliosides in the lysosome. Mutations in the β-hexosaminidase A α-subunit can cause extensive ERAD of HexA, leading to Tay-Sachs disease (TSD). Jeyakumar et al., Neuropathol Appl Neurobiol 28: 343-357, 2002. HexA activity was studied in a compound heterozygous fibroblast cell line (GM13204), harboring one of the most prevalent β-hexosaminidase A α-subunit mutations (G269S) found in Tay-Sachs patients and a second mutated HexA allele (1278insTATC) with a stop codon. Activity of the α-site within the HexA enzyme was measured using the MUGS substrate revealing that untreated G269S HexA fibroblasts have 10% of the WT Hex α-site activity. Tropak et al., J Biol Chem 279: 13478-13487, 2004. MG-132 administration (0.8 to 1 μM) led to a G269S Hex α-site activity increase of 1.8-fold (to ≈18% of cellular WT Hex α-site activity) after 144 h incubation period (FIG. 4A), while celastrol (0.4 to 0.6 μM) afforded a 1.6-fold increase (to ≈16% of cellular WT HexA α-site activity) after 96 h of incubation (FIG. 4B).

### Example 7

### Proteostasis Regulator and Pharmacologic Chaperone Synergy in Tay-Sachs Disease

2-Acetamido-2-deoxynojirimycin (ADNJ) has been reported to function as a pharmacologic chaperone in a number of Tay-Sachs patient-derived cell lines. Tropak et al., Biol Chem 279: 13478-13487, 2004. The compound heterozygous G269S/1278insTATC HexA fibroblast cell line was exposed once to ADNJ without media changes (20 to 50 μM), affording a 2.5-fold (150 unit) increase in cellular Hex α-site activity (to 25% of cellular WT Hex α-site activity) after 192 h (FIG. 4C). Based on the Gaucher disease examples described above, we expected that the co-administration of the proteostasis regulator (MG-132) and the pharmacologic chaperone (ADNJ) would lead to an enhanced rescue of Hex α-site activity. A 5-fold (400 unit) increase in G269S Hex α-site activity (to ≈50% of cellular WT Hex α-site activity) was detected 144 h after a single exposure to MG-132 (0.8 μM) and ADNJ (20 μM) (FIGS. 4D and 15A and 15B), which is greater than the 3.3-fold (230 unit) sum of the individual effects, demonstrating synergy. Dosing regimen optimization should further enhance the substantial activity increase observed.

FIG. 4 shows proteostasis regulator alone, or in combination with an enzyme-specific pharmacologic chaperone, enhances Hex α-site activity of a G269S/1278insTATC HexA Tay-Sachs fibroblast cell line. G269S/1278insTATC HexA fibroblasts were exposed to A) 0.2 to 1.2 μM MG-132 and the Hex α-site activities measured at 96, 120, 144, 168, 192 h; B) 0.2 to 1.2 μM celastrol and the Hex α-site activities measured at 24, 48, 72, 96, 120 h; C) 5 to 100 μM ADNJ and the Hex α-site activities measured at 96, 120, 144, 168, 192 h. All activities were reported relative to the activity of untreated cells of the same type (left y axis) and as the percentage of WT Hex α-site activity (right y axis). D) Hex α-site activity of G269S/1278insTATC HexA fibroblast cell line exposed to MG-132 and ADNJ in the media at t=0. The 3D plot depicts the MG-132 and ADNJ media concentrations on the x and y-axes, and the Hex α-site activity on the z-axis, measured after 144 h of growth, relative to the activity of untreated cells of the same type (left z axis) and as the percentage of WT Hex α-site activity (right z axis). 2D plots of relative Hex α-site activity in G269S/1278insTATC HexA cells vs. ADNJ and MG-132 concentrations are reported in FIGS. 15A and 15B, respectively.

### Example 8

### Insight into the Mechanism of Action of MG-132 and Celastrol

It has been previously demonstrated that celastrol and MG-132 induce the heat-shock response (HSR), enhancing the expression of heat shock proteins in the cytosol. Bush et al., J Biol Chem 272: 9086-9092, 1997; Liao et al., J Cell Biochem 99: 1085-1095, 2006; Westerheide et al., J Biol Chem 279: 56053-56060, 2004. It may at first be surprising that the heat shock response could have a substantial effect on the proteostasis of GC folded and trafficked within the secretory pathway as a soluble lumenal enzyme. However, upon further reflection, cytosolic factors including chaperones are likely essential for adapting the secretory pathway to be more folding and trafficking permissive. In addition, MG-132 and celastrol may also induce one or more of the three arms of the unfolded protein response (UPR) that remodels the secretory pathway, especially the ER, to be more folding and export permissive. Ron et al., Nat Rev Mol Cell Biol 8: 519-529, 2007; Schroeder et al., Ann Rev Biochem 74: 739-789, 2005.

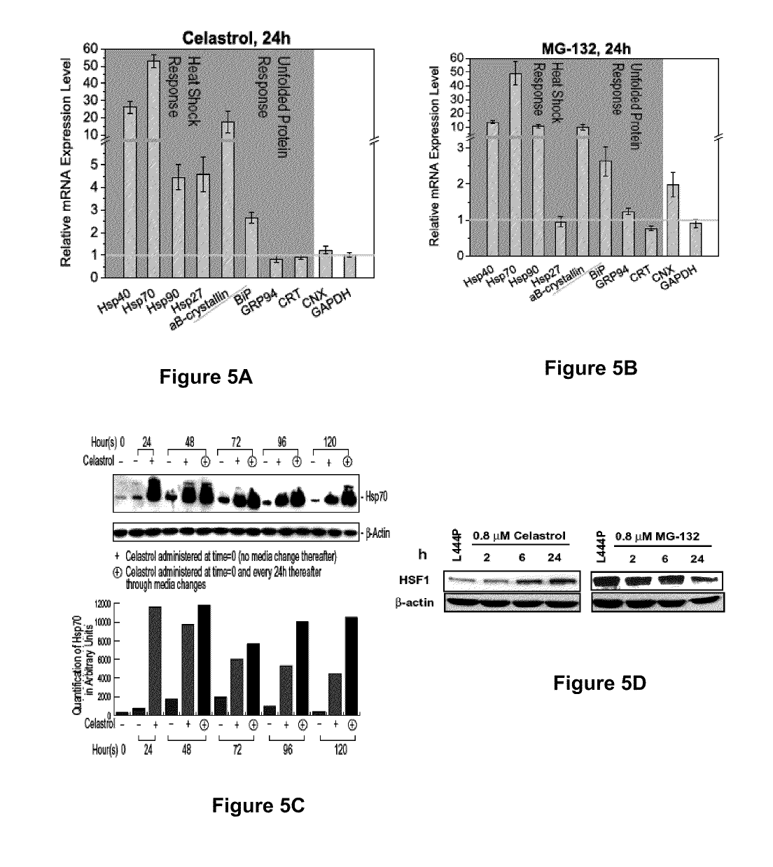

To monitor to what extent the HSR and the UPR were induced by celastrol and MG-132 treatment, quantitative reverse transcription-polymerase chain reaction (RT-PCR) analysis was performed (primers listed in Table 2). L444P GC cells were incubated alone, with 0.8 μM MG-132, or with 0.8 μM celastrol for 24 h prior to total RNA extraction. The relative mRNA expression levels of representative cytoplasmic HSR-associated chaperones (Hsp40, Hsp70, Hsp90, Hsp27, αB-crystallin), ER lumenal UPR-associated chaperones (BiP, GRP94, calreticulin), and the ER chaperone calnexin were monitored and their reported levels were normalized to the levels in untreated cells (FIGS. 5A and B). The levels of glyceraldehyde-3-phosphate dehydrogenase, monitored as a housekeeping control, were unchanged in treated and untreated cells.

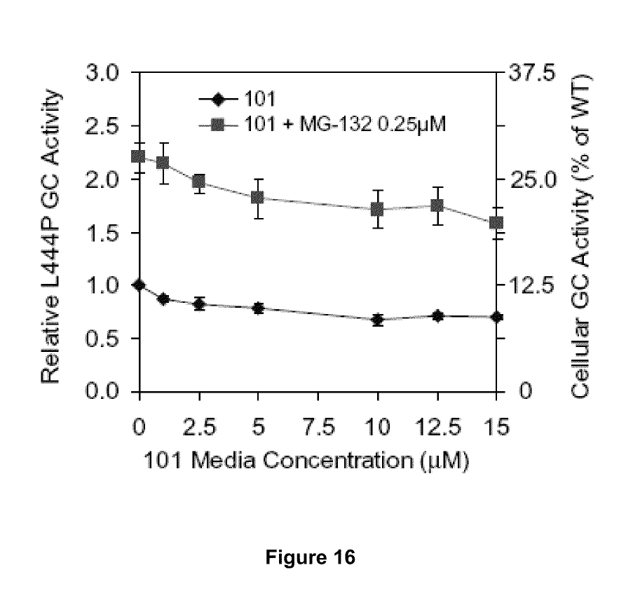

Treatment with either proteostasis regulator upregulates both cytoplasmic and ER lumenal chaperones. Both proteostasis regulators significantly upregulated the mRNA expression levels of Hsp40, Hsp70, and Hsp90, αB-crystallin in the cytosol and BiP in the ER lumen and neither altered transcription of GRP94 and calreticulin (FIGS. 5A and B). However, there are also differences. Celastrol increased transcription of Hsp27 significantly, whereas MG-132 treatment did not. Conversely, MG-132 treatment upregulated the transcription of calnexin significantly, but celastrol treatment did not. The 50-fold upregulation of Hsp70 suggests that this chaperone might be particularly important in the partial restoration of mutant GC function. Addition of the HSP70 inhibitor 101 to untreated L444P GC fibroblasts resulted in reduced L444P GC activity (FIG. 16). Moreover the co-administration of this inhibitor with MG-132 antagonized the enhancement of L444P GC activity by MG-132 (FIG. 16), supporting the idea that HSP70, a cytosolic chaperone, is an important chaperone in GC proteostasis in fibroblasts.

FIG. 16. Effect of Compound 101, an Hsp70 inhibitor alone, or in combination with MG-132 on GC activity in L444P GC fibroblasts. Compound 101 was applied without or with 0.25 μM MG-132 for 24 h, 10 and L444P GC activity was assayed, normalized against that of untreated L444P GC cells (left y axis), and expressed as the percentage of WT GC activity (right y axis).

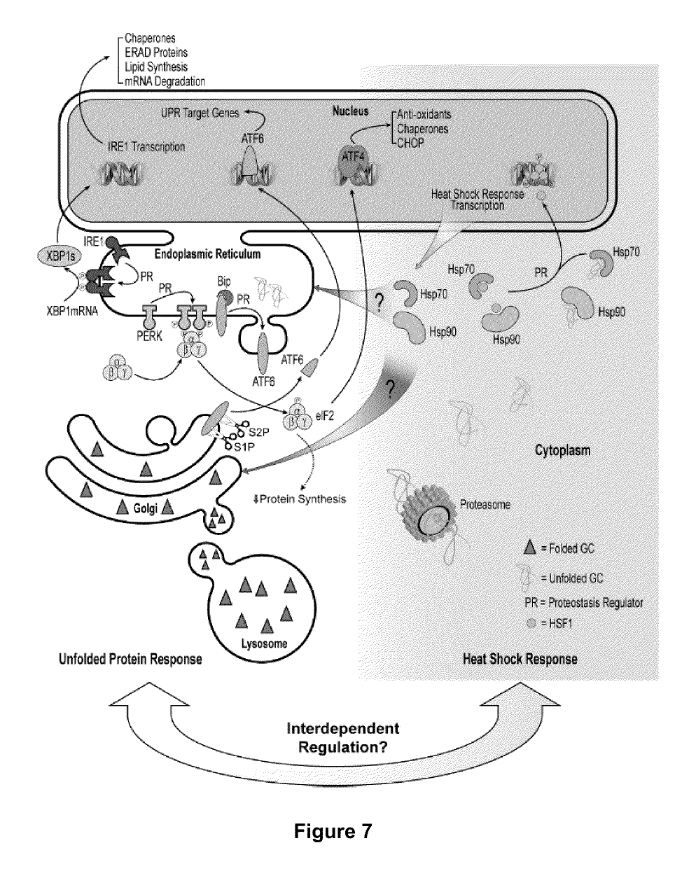

To correlate changes in Hsp70 chaperone levels with the celastrol-mediated transcriptional increase in Hsp70, as well as to investigate the temporal dependence of the change in Hsp70 levels, L444P GC fibroblasts were subjected to a single celastrol exposure (0.8 μM) for the period indicated and Hsp70 expression levels in cell lysates were analyzed by Western blot analysis. Hsp70 levels were considerably higher in celastrol treated cells (FIG. 5C, red bars) than in untreated cells (blue bars) and were maximal after 24 h contributing to the peak in L444P GC activity at 120 h (FIG. 1A). Strictly analogous results were observed for N370S and G202R GC fibroblasts. Untreated L444P GC cells (FIG. 5C, blue bars) exhibit only modestly enhanced Hsp70 levels at 48, 72 and 96 h, explaining at least in part the enhanced GC levels in the absence of celastrol exposure observed in FIG. 1B after 72 and especially after 120 h, although these increased GC and Hsp70 levels did not result in a measurable increase in GC activity. Hsp70, a product of HSR activation, has been implicated in HSR autoregulation by binding to HSF1, thereby repressing heat shock gene transcription (FIG. 7). Morimoto, Genes Dev 12: 3788-3796, 1998; Westerheide et al., J Biol Chem 280: 33097-33100, 2005. Therefore, fibroblasts exposed to celastrol once (0.8 μM) or every 24 h for 96 h showed decreased levels of Hsp70 expression with time, consistent with autoregulation of the HSR (FIG. 5C).

Since HSF1 is likely to be responsible for celastrol's induction of the HSR we monitored HSF1 levels. Westerheide et al., J Biol Chem 279: 56053-56060, 2004. HSF1 levels in L444P GC fibroblasts after treatment with celastrol or MG-132 for the indicated period were monitored by Western blot analysis (FIG. 5D). As reported, celastrol increased HSF1 levels over the 24 h period while HSF1 levels remained constant with MG-132 treatment over the same time course, consistent with reports that MG-132 induces the HSR without significantly upregulating HSF1. Awasthi, et al., Invest Opthalmol Vis Sci 46: 2082-2091, 2005; Bush et al., J Biol Chem 272: 9086-9092, 1997.

FIG. 5 shows both MG-132 and celastrol activate the heat shock response in L444P GC fibroblasts. Relative chaperone mRNA expression levels probed by quantitative RT-PCR in 0.8 μM celastrol (A) or 0.8 μM MG-132 (B) treated L444P GC fibroblasts. L444P GC cells were incubated with the drug for 24 h. Relative mRNA expression level for treated L444P GC cells was normalized to that of untreated cells after corrected to the expression level of GAPDH, a housekeeping control. C) Western blot analysis of Hsp70 levels in L444P GC cells with (+) and without (−) celastrol as a function of time. Over 120 h, aliquots of cells were lysed every 24 h to extract proteins, and an equal amount of protein was loaded. β-actin served as a gel loading control. Hsp70 bands from cells never exposed to celastrol (−), exposed to celastrol at t=0 (+), or also exposed to celastrol (0.8 μM) at t=0 with media changes every 24 h (⊕), were quantified as described in FIG. 1B. D) HSF1 protein expression level in celastrol and MG-132 treated L444P GC cells. L444P GC cells were treated with 0.8 μM celastrol and 0.8 μM MG-132 for the indicated amount of time before being lysed for SDS-PAGE analysis. HSF1 was probed using western blot analysis. β-actin served as a loading control.

### Example 9

### Celastrol and Mg-132 Treatment Induces the Unfolded Protein Response (UPR)

The ER responds to the accumulation of unfolded proteins in its lumen by activating up to three integrated arms of intracellular signaling pathways, collectively referred to as the unfolded protein response, that regulate the expression of numerous genes that function within the secretory pathway. Ron et al., Nat Rev Mol Cell Biol 8: 519-529, 2007; Schroeder et al., Ann Rev Biochem 74: 739-789, 2005. To explore whether the UPR was activated upon celastrol or MG-132 treatment, we monitored three well-established UPR-associated stress sensors: IRE1, ATF6, and PERK, integral membrane proteins that can sense folding status in the ER and transmit a signal across the ER membrane to the cytoplasm and into the nucleus, ultimately resulting in transcriptional activation. Ron et al., Nat Rev Mol Cell Biol 8: 519-529, 2007; Schroeder et al., Ann Rev Biochem 74: 739-789, 2005.

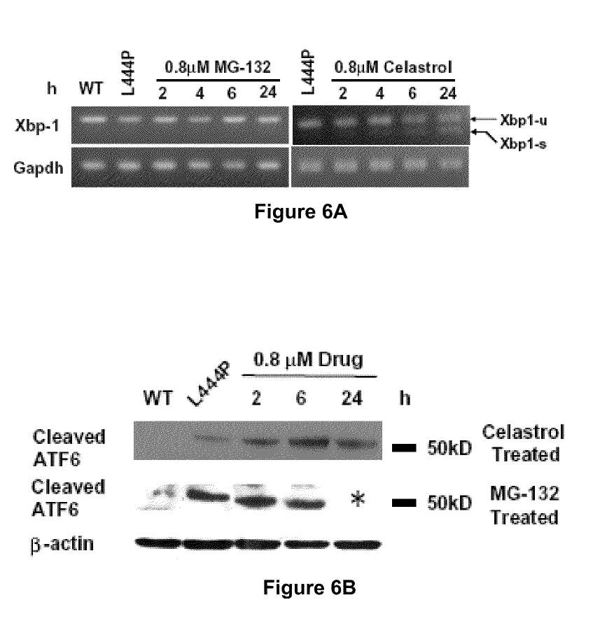

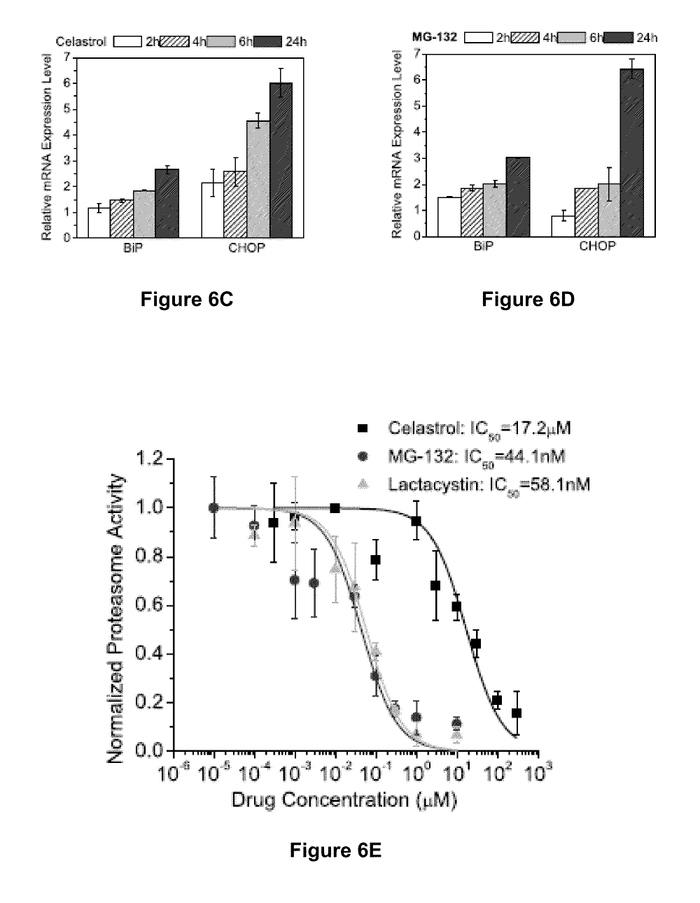

IRE1 responds to stress by oligomerization, resulting in trans-autophosphorylation that activates its endonuclease function, precisely splicing the mRNA that encodes the transcription factor XBP1. Ron et al., Nat Rev Mol Cell Biol 8: 519-529, 2007; Schroeder et al., Ann Rev Biochem 74: 739-789, 2005. RT-PCR was performed to detect the spliced mRNA of XBP1. The spliced form of Xbp-1 appeared over the period of 4 to 24 h upon a single exposure of L444P GC fibroblasts to celastrol (0.8 μM), indicating the activation of the IRE1 arm of the UPR (FIG. 6A). In contrast, no spliced Xbp-1 could be detected in L444P GC cells upon a single exposure to MG-132 (FIG. 6A), indicating that IRE1 was not activated during this time period. No Xbp-1 splicing was observed in WT fibroblasts either, as expected.

ATF6 responds to stress by regulated proteolysis in the Golgi, liberating a cytosolic fragment of ATF6 that activates a subset of UPR genes. Ron et al., Nat Rev Mol Cell Biol 8: 519-529, 2007; Schroeder et al., Ann Rev Biochem 74: 739-789, 2005. Cleavage of ATF6 was monitored by Western blot analysis. A significant amount of the cleaved and activated 50 kD form of ATF6 was observed in untreated L444P GC fibroblasts, while none was detected in untreated WT cells (FIG. 6B). Our observation that ATF6 is constitutively activated in L444P GC fibroblasts is consistent with recent reports that ATF6a optimizes long term ER function to protect cells from chronic stress. Wu et al., Dev Cell 13: 351-364, 2007. Application of celastrol or MG132 (0.8 μM) for 2 h increased the level of cleaved ATF6, indicating activation of the ATF6 arm of the UPR. Incubation with celastrol or MG132 for longer (24 h) diminished the activation of AFT6.

PERK responds to stress by oligomerizing and phosphorylating the α subunit of eIF2, which leads to the ATF4-mediated production of CHOP and other proteins, including chaperones. Ron et al., Nat Rev Mol Cell Biol 8: 519-529, 2007; Schroeder et al., Ann Rev Biochem 74: 739-789, 2005. MG-132 or celastrol (0.8 μM) treatment upregulated the mRNA expression level of CHOP significantly, as discerned by quantitative RT-PCR analysis (FIGS. 6C and D, right panels). BiP was also upregulated in both MG-132 or celastrol (0.8 μM)-treated L444P GC fibroblasts (FIGS. 6C and D, left panels). BiP is thought to be cytoprotective whereas CHOP can lead to apoptosis through mechanisms that are not well understood. Ron et al., Nat Rev Mol Cell Biol 8: 519-529, 2007; Schroeder et al., Ann Rev Biochem 74: 739-789, 2005. Although PERK activation can lead to attenuation of global protein synthesis, both celastrol and MG-132 administration to L444P GC fibroblasts resulted in an increase in GC levels (FIGS. 1B, 2B).

Since the UPR and HSR are activated by both celastrol and MG-132, we used small interfering RNA (siRNA) treatment to discern the stress-associated signaling pathway(s) that are functionally important for L444P GC PR activity. siRNAs against HSF1 (responsible for the HSR) or IRE1α or ATF6 or PERK (the three arms of the UPR) were coadministered one at a time along with a PR. The experiment involves a 24 hr siRNA pretreatment of L444P GC fibroblasts followed by 24 hr of PR treatment in DMSO (along with DMSO vehicle controls). Western blot analysis revealed that HSF1, IRE1α, and ATF6 were silenced for 48 hr after the transfection with corresponding siRNA compared to a mock transfection or a negative control utilizing nontargeting siRNA (not shown). Gene knockdown of HSF1, ATF6, and PERK for at least 48 hr after transfection was also verified by quantitative RT-PCR analysis (not shown).

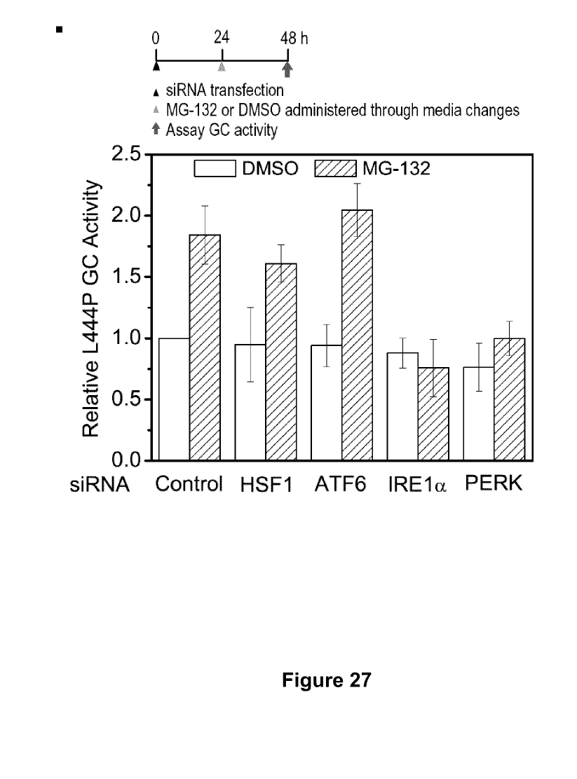

L444P GC fibroblasts were treated with the corresponding siRNA for 24 hr and then MG-132 (0.25 μM in DMSO) for another 24 hr before the intact cell GC activity assay or lysis for western blot analysis was performed. L444P GC activity was increased when MG-132 was coapplied with nontargeting control siRNA (FIG. 5C). Coapplication of either HSF1 siRNA or ATF6 siRNA did not significantly diminish the GC activity increase afforded by MG-132 treatment (FIG. 27), indicating that HSF1 and ATF6 are not required for MG-132 PR function. Coapplication of either IRE1α siRNA or PERK siRNA with MG-132 significantly diminished the GC activity increase (FIG. 27), indicating that the IRE1α and PERK UPR arms are important for MG-132 PR function.

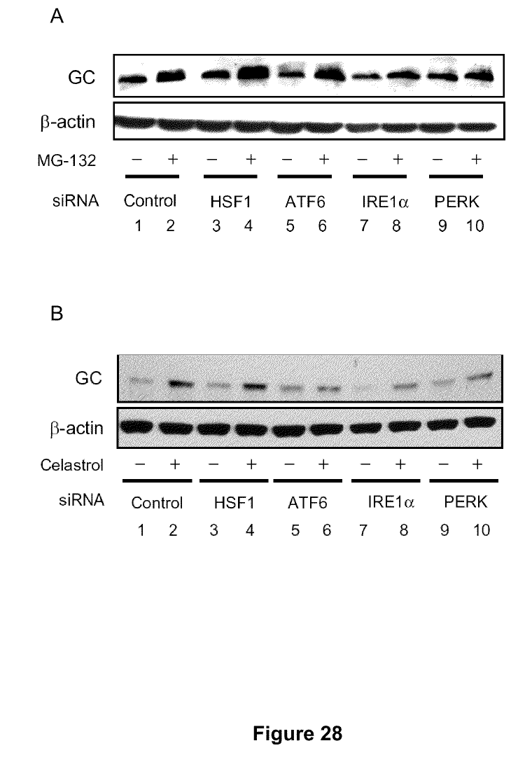

GC western blot analysis confirmed these observations. MG-132 increased the GC band intensity significantly when nontargeting control siRNA was coapplied (FIG. 28A, cf. lanes 1 and 2). Coapplication of MG-132 and either HSF1 siRNA or ATF6 siRNA did not significantly diminish the GC band intensity increase (FIG. 28A, cf. lanes 3 and 4, and lanes 5 and 6). In contrast, coapplication of either IRE1α siRNA and MG-132 or PERK siRNA and MG-132 significantly diminished the L444P GC band intensity increase (FIG. 28A, cf. lanes 7 and 8, and lanes 9 and 10), consistent with the conclusion drawn above that IRE1α and PERK are required for MG-132 L444P GC PR function.

Western blot analysis of L444P GC levels demonstrated that coapplication of celastrol and siRNAs directed against ATF6, IRE1α, and PERK, but not HSF1 or nontargeting control siRNA, partially blocked celastrol's L444P GC PR function (FIG. 28B). As with MG-132, the UPR appears to be critical for mediating the PR function of celastrol, although in the case of celastrol, all three arms of the UPR appear to be important.

In summary, celastrol clearly activates all three arms of the UPR, whereas MG-132 appears to use the ATF6 and PERK arms, but not the IRE1 arm.

### Example 10

### Proteasome Inhibition does not Appear to be Sufficient for GC Proteostasis Regulator Function

Ang et. al published evidence that celastrol can also act as a proteasome inhibitor in vitro and in cell culture, suggesting that its role as a proteostasis regulator could also be influenced by this activity, as it had already been established that proteasome inhibition enhances the expression levels of numerous chaperones. Yang et al., Cancer Res 66: 4758-4765, 2006; Bush et al., J Biol Chem 272: 9086-9092, 1997; Liao et al., J Cell Biochem 99: 1085-1095, 2006. L444P cells were incubated with the proteasome inhibitors celastrol, MG-132, or lactacystin as a function of concentration for 2 h before measurement of the chymotrypsin-like activity of the proteasome. MG-132, lactacystin, and celastrol exhibited a half maximal inhibitory concentration (IC₅₀) value of 44.1±5.4 nM, 58.1±6.4 nM, and 17.2±2.1 μM, respectively (FIG. 6E). Celastrol hardly inhibits the chymotrypsin-like activity of the proteasome at the 0.8 μM concentration used in these studies (FIG. 6E), making it unlikely that its proteostasis regulator activity is principally mediated through the proteasome. While it is clear that MG-132 inhibits the chymotrypsin-like activity of the proteasome at the 0.8 μM concentration employed, the nearly exact dose response curve of the more selective proteasome inhibitor lactacystin, which is not a GC proteostasis regulator, suggests that inhibiting the chymotrypsin-like activity of the proteasome is not sufficient for GC proteostasis regulator function. MG-132 contains an aldehyde functionality, which is known to inhibit other proteases. Thus, one possibility is that the activity of an unknown protease contributes to its GC proteostasis regulator function as well. Consistent with this hypothesis, MG-132 also influences CFTR maturation in a fashion distinct from lactacystin. Jensen et al., Cell 83: 129-135, 1995.

FIG. 6 shows GC proteostasis regulation by MG-132 and celastrol might occur through the unfolded folded protein response. A) Detection of spliced Xbp-1 mRNA by RT-PCR in 0.8 μM MG-132 or 0.8 μM celastrol treated L444P GC fibroblasts for 2, 4, 6, and 24 h. WT cells were also probed as a control and GAPDH was used as a housekeeping control. Xbp1-u represents unspliced Xbp-1, a 289 bp amplicon, and Xbp1-s represents spliced Xbp-1, a 263 bp amplicon. B) Cleavage of ATF6 in celastrol or MG-132 treated L444P GC cells. L444P GC cells were untreated or treated with 0.8 μM celastrol or 0.8 μM MG-132 for 2, 6 and 24 h before being lysed for SDS-PAGE analysis. WT cells served as a control. ATF6 was probed using western blot analysis. β-actin served as a loading control. * Cleaved ATF6 was undetectable after 24 h treatment with MG-132 in 3 separate experiments. Relative mRNA expression levels of BiP and CHOP probed by quantitative RT-PCR in celastrol (C) or MG-132 (D) treated L444P GC fibroblasts. L444P GC cells were untreated or incubated with 0.8 μM celastrol (C) or MG-132 (D) for 2, 4, 6, and 24 h. Relative mRNA expression level for treated L444P GC cells was normalized to that of untreated cells after correction for the expression level of GAPDH, a housekeeping control. E) Inhibition of chymotrypsin-like activity of the proteasome by celastrol, MG-132, or lactacystin in L444P GC cells. The L444P cells were incubated with celastrol, MG-132, or lactacystin at various concentrations for 2 h before cell-based assay was performed to measure chymotrypsin-like activity of the proteasome.

### Example 11

### Proteostasis Restoration for Treatment of Disease

The present results have demonstrated that it is feasible to adapt the cellular proteostasis network to fold, traffic, and restore function to mutated enzymes that would otherwise be degraded and lead to loss-of-function diseases. Partial restoration of proteostasis, enabled by small molecule proteostasis regulators that transcriptionally activate at least a subset of the HSR and UPR genes, is an appealing strategy to treat loss-of-function diseases because one molecule can be used for more than one disease, as the proteostasis network has evolved to handle thousands of proteins simultaneously. The substantial influence of cytoplasmic chaperones, including Hsp70, for enhancing the folding and trafficking capacity of the secretory pathway has important implications, one of which is that there may be more interdependent regulation between the UPR and the HSR than currently appreciated (FIG. 7).

FIG. 7 shows GC proteostasis restoration pathways. The proteostasis regulators celastrol and MG-132 activate both the heat shock response and the unfolded protein response, which may be interdependently regulated. A direct consequence of these responses is the upregulation of molecular chaperones that help folding and trafficking, and minimize the degradation of mutant enzymes.

The demonstration that one proteostasis regulator can be used to restore partial enzyme function in two distinct LSD cell lines harboring non-homologous mutated misfolding-prone enzymes that perform different chemistry is appealing. There are more than 40 different LSDs and PR have wide-ranging effectiveness against proteostasis-related conditions by virtue of the common folding, trafficking and/or other pathways through which many PR exert a therapeutic effect. Beutler et al., Mol Genet Metab 88: 208-215, 2006; Jeyakumar et al., Neuropathol Appl Neurobiol 28: 343-357, 2002. MG-132 and celastrol each partially restore folding, trafficking, and function to two different mutated glycolipid degrading enzymes (glucocerebrosidase and hexosaminidase A, in patient derived Gaucher and Tay-Sachs cell lines) and the results suggest they would also be effective against other LSD-associated mutant misfolding-prone enzymes.

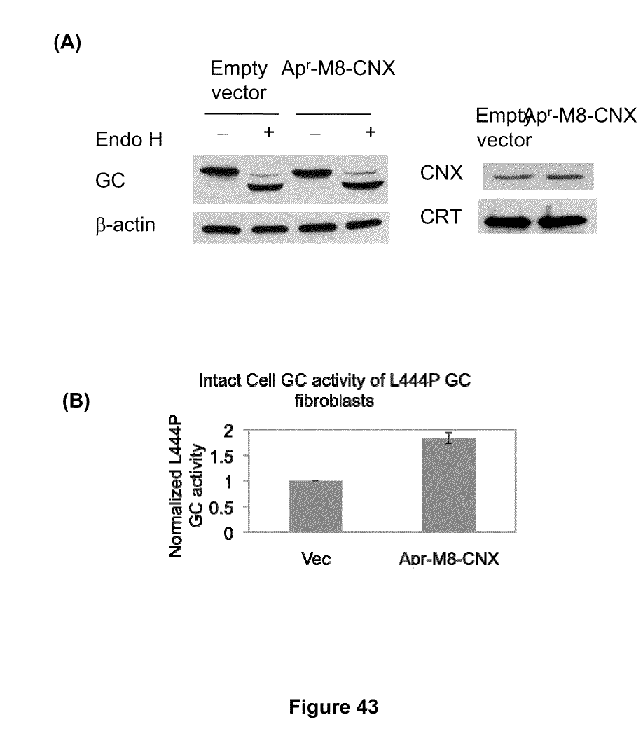

Pharmacologic chaperones bind to and stabilize the folded conformational ensemble of a given misfolding-prone protein, increasing the population that can engage the export machinery, and thus increasing its population in the destination environment (FIG. 3A). In LSDs, it is straightforward to discover pharmacologic chaperones for misfolding-prone enzymes because one can often simply use enzyme inhibitors, at sub-inhibitory concentrations. Several pharmacologic chaperones are now in clinical trials for specific LSDs, including Gaucher and Fabry disease. Fan et al., Nat Med 5: 112-115, 1999; Sawkar et al, Cell Mol Life Sci 63: 1179-1192, 2006a; Yu et al., FEBS Lett 274: 4944-4950, 2007a. To test whether the effect of chaperones on protein folding is sufficient to restore loss-of-function in a LSD, the ER-associated chaperone calnexin was overexpressed in L444P GC fibroblasts and the GC activity (FIG. 43B) and GC glycosylation pattern (FIG. 43A) were measured. Calnexin significantly enhanced GC activity and yielded a glycosylation pattern indicating that the proportion of active, fully folded and glycosylated GC is enhanced in the presence of calnexin.

Unlike pharmacologic chaperones that stabilize the folded state of a given protein, proteostasis regulators that transcriptionally activate the HSR and the UPR, or components thereof, work by enhancing the efficiency by which protein folding intermediates progress to the folded state while minimizing competing aggregation (FIG. 3A). Thus, pharmacologic chaperones and proteostasis regulators work through largely distinct mechanisms (FIG. 3A), explaining why we observe synergistic increases in lysosomal enzyme function in the refractory L444P GC neuropathic Gaucher cell line (as well as N370S and G202R GC fibroblast cell lines) and the G269S Tay-Sachs cell line with the co-administration of a proteostasis regulator and an enzyme specific pharmacologic chaperone.

In summary, the present results have shown that two proteostasis regulators each transcriptionally activate both the HSR and the UPR and partially restore glucocerebrosidase and β-hexosaminidase A homeostasis in Gaucher and Tay-Sachs disease patient derived cell lines, respectively. This demonstrates that it is possible to treat more than one LSD with a single proteostasis regulator. Moreover, the present results demonstrate that the combined use of a proteostasis regulator and an active site directed pharmacologic chaperone yields synergistic restoration of mutant enzyme function in Gaucher and Tay-Sachs disease patient derived fibroblasts. Optimization of the chemistry and biology of these proteostasis regulators and their dosing schedules, discovery of additional proteostasis regulators, as well as enhancing the dosing strategies for the combined use of pharmacologic chaperones and proteostasis regulators, or two distinct proteostasis regulators, offer the promise of yielding clinical candidates for LSDs and possibly other loss-of-function diseases.

### Example 12

### Experimental Procedures

Reagents. Celastrol, MG-132, PS I, PSIV, Tyropentin A, and lactacystin were from Calbiochem (San Diego, Calif.). N-(n-nonyl)deoxynojirimycin (NN-DNJ), 2-acetamido-2-deoxynojirimycin (ADNJ), 4-Methylumbelliferyl 6-Sulfo-2-acetamido-2-deoxy-β-D-glucopyranoside (MUGS), Conduritol B Epoxide (CBE) were from Toronto Research Chemicals (Downsview, ON, Canada). 4-methylumbelliferyl β-D-glucoside (MUG) was from Sigma (St. Louis, Mo.). D-glucosyl-β1-1′-N-dodecanoyl-D-erythro-sphingosine (C12 β-D-glucosyl ceramide) and N-lauroyl-D-erythro-sphingosine (C12 ceramide) were from Avanti Polar Lipids (Alabaster, Ala.). The Hsp70 inhibitor Compound 101 was a kind gift from Professor Jeffrey Brodsky (University of Pittsburgh, Pittsburgh, Pa.). Cell culture media were purchased from Gibco (Grand Island, N.Y.).

Cell cultures. Primary skin fibroblast cultures were established from patients homozygous for the G202R (c.721G>A) and the N370S (c.1226A>G) mutations. Wild type primary skin fibroblasts (GM05659, GM00498), the GD fibroblast cell line homozygous for the L444P (c. 1448T>C) mutation (GM08760), and the TSD fibroblast cell line heterozygous for the G269S (c.805G>A) mutation and a 4 base pair insertion (c.1278insTATC) (GM13204) were obtained from Coriell Cell Repositories (Camden, N.J.). Fibroblasts were grown in minimal essential medium with Earle's salts supplemented with 10% heat-inactivated fetal bovine serum and 1% glutamine Pen-Strep at 37° C. in 5% CO₂. Cell medium was replaced every 3 or 4 days. Monolayers were passaged upon reaching confluency with TrypLE Express.

Enzyme activity assays. The intact cell GC activity assay has been previously described. Sawkar et al., Proc Natl Acad Sci USA 99: 15428-15433, 2002. Briefly, approximately 10⁴ cells were plated in each well of a 96-well plate (100 μl per well) overnight to allow cell attachment. Medium was replaced with fresh medium containing small molecules and plates were incubated at 37° C. Trypan blue staining was utilized to measure cell viability after drug treatment. The medium was then removed and monolayers washed with PBS. The assay reaction was started by the addition of 50 μl of 2.5 mM MUG in 0.2 M acetate buffer (pH 4.0) to each well. Plates were incubated at 37° C. for 7 hours and the reaction was stopped by the addition of 150 μl of 0.2 M glycine buffer (pH 10.8) to each well. Liberated 4-methylumbelliferone was measured (excitation 365 nm, emission 445 nm) with a SpectraMax Gemini plate reader (Molecular Device, Sunnyvale, Calif.). Control experiments to evaluate the extent of unspecific non-lysosomal GC activity were performed by adding CBE to the assay reaction. Typically, culture medium was replaced with medium containing small molecule after overnight incubation (time 0). Alternatively, when L444P GC fibroblasts were incubated with celastrol or with NN-DNJ and celastrol, after adding the compounds at time 0, the medium was replaced every 24 hours (or as indicated in the Results and Figures for each specific experiment) with fresh medium containing the same compound concentration that was originally present in each well, as described in the Results section. GC activities measured were normalized to the corresponding protein concentration for each sample.

The Hex α-site cell assay has been previously described. Tropak et al., J Biol Chem 279: 13478-13487, 2004. Cells were plated as described for the GC assay. After 1 to 8 days of incubation the medium was removed, cells were washed with PBS, and lysed with 60 μl of 10 mM citrate/phosphate buffer pH 4.2 (CP buffer) containing 0.5% human serum albumin and 0.5% Triton X-100. 30 μl of aliquots were transferred to a 96-well plate and Hex α-site activity was measured by adding 30 μl of 3.2 mM MUGS in CP buffer to each well and incubating the plates at 37° C. for 1 to 7 hour. The reaction was stopped by adding 200 μl of 0.1 M 2-amino-2-methyl-1-propanol pH 10.5 and the fluorescence was measured (excitation 365 nm, emission 450 nm).

For both the Hex α-site and the GC activity assays each data point reported was evaluated at least in triplicate in each plate, and on three different days. The data reported were normalized to the activity of untreated cells, and expressed as the percentage of WT enzyme activity for each different cell line.

Degradation of a natural GC substrate. A variety of cell lines harboring WT and variant GC were lysed with the complete lysis-M buffer containing complete protease inhibitor cocktail (Roche, Nutley, N.J.). 30 μg of total protein was incubated in 50 μl of 0.1 M acetate buffer (pH 5.0) containing 1 mg/ml C12 β-D-glucosyl ceramide, a natural GC substrate, in the presence of 0.15% Triton X-100 (v/v, Fisher) and 0.15% taurodeoxycholate (w/v, Calbiochem) at 37° C. The degradation reaction of C12 β-D-glucosyl ceramide to C12 ceramide was monitored by thin layer chromatography (TLC) developed in the solvent of methanol/dichloromethane (1:9), and visualized by iodine staining. Conversion of the spot with an R_{f} value of 0.25 (corresponding to C12 β-D-glucosyl ceramide) to the spot with an R_{f} value of 0.52 (corresponding to C12 ceramide) indicates the degradation of the natural substrate. The experiments were performed three times and similar results were obtained.

Western blot analyses. Cells were lysed with the complete lysis-M buffer containing complete protease inhibitor cocktail (Roche, Nutley, N.J.). Total cell protein was determined with Micro BCA assay reagent (Pierce, Rockford, Ill.) and each sample was diluted to the same protein concentration. Company specifications were followed for protein treatment with EndoH and PNGase F (New England Biolabs, Ipswich, Mass.). Aliquots of cell lysates were separated in a 10% SDS-PAGE gel and western blot analysis was performed using appropriate antibodies. Rabbit anti-Hsp70, anti-HSF1, and anti-actin were from Stressgen (Victoria, BC, Canada). Mouse monoclonal anti-GC 2E2 was from Novus Biologicals (Littleton, Colo.). Mouse monoclonal anti-ATF6 was from IMGENEX (San Diego, Calif.). Secondary goat anti-rabbit and goat anti-mouse HRP-conjugated antibodies were from Pierce. Blots were visualized using SuperSignal West Femto Maximum Sensitivity or West Pico Substrate (Pierce). The western blot bands of the endoH treated samples were quantified by Java Image processing and analysis software from the NIH (http://rsb.info.nih.gov/ij/).

Cell-based chymotrypsin-like proteasomal activity assay. Proteasome-Glo Cell-Based Assay kit (Promega, Madison, Wis.) was utilized to measure the chymotrypin-like proteasomal activity. Briefly, approximately 5×10³ L444P GC cells were plated in each well of a 96-well plate (100 μl per well) overnight to allow cell attachment. Medium was replaced with fresh medium containing proteasome inhibitors at various concentrations. After 2 h incubation at 37° C., following the company's instruction, 100 μl/well of Proteasome-Glo Cell-Based reagent was added. Luminescence was measured with a SpectraMax Gemini plate reader. The luminescence of treated cells was normalized to that of untreated cells after background subtraction. IC₅₀ values were calculated by fitting the data to the formula: y=IC₅₀/(IC₅₀+x), where y is the normalized luminescence signal, and x is the inhibitor concentration. Each data point was evaluated at least in triplicate, and on three different days. The data reported was expressed as IC₅₀±SD in the text.

Immunofluorescence. Immunofluorescence has been previously described. Sawkar et al., ACS Chem Biol 1: 235-251, 2006. Briefly, cells grown on cover glass slips were fixed with 3.7% paraformaldehyde in PBS for 15 min. The cover slips were washed with PBS, quenched with 15 mM glycine in PBS for 10 min, and permeabilized with 0.2% saponin in PBS for 15 min. The antibodies were prepared in PBS in the presence of 0.2% saponin and 5% goat serum. Cells were incubated for 1 hour with primary antibodies (1:100 for mouse monoclonal anti-GC 8E4, and 1:10,000 for rabbit anti-LAMP2, washed with 5% goat serum in PBS, and then incubated for 1 hour with secondary antibodies (Alexa Fluor 488 goat anti-mouse IgG and Alexa Fluor 546 goat anti-rabbit IgG from Molecular Probes (Eugene, Oreg.). The cover slips were mounted and sealed. Images were collected using a Bio-Rad (Zeiss) Radiance 2100 Rainbow laser scanning confocal microscope attached to a Nikon TE2000-U microscope, and analyzed using NIH Image J software. The experiments were done three times and similar results were obtained.

Relative quantification of protein expression level changes by Multidimensional Protein Identification Technology (MudPIT). Proteins from each sample were precipitated using 25% trichloroacetic acid (v/v) and ice-cold acetone. The pellet was air-dried and suspended with 8 M urea containing 1× Invitrosol (Invitrogen, Carlsbad, Calif.) in 100 mM Tris-HCl pH 8.5. The protein concentration was measured using the BCA Protein Assay Kit (Pierce). An amount of 200 μg of total protein was first reduced by incubating with Tris(2-carboxyethyl) phosphine (TCEP) at 5 mM for 30 min, and then alkylated by incubating with iodoacetamide (IAA) at 10 mM for 20 min in the dark. The samples were subsequently diluted to 2 M urea with 100 mM Tris-HCl, pH 8.5, brought to 1 mM CaCl₂, and digested by adding sequence grade modified trypsin (Promega, Madison, Wis.) at an enzyme/substrate ratio of 1:30 and incubating overnight at 37° C. The digestion reaction was quenched by adding formic acid to 5% (v/v) to lower the pH to 2-3. Samples not immediately analyzed were stored at −80° C. For each sample, three replicates of 60 μg of the protein digest were analyzed each time by MudPIT (Link et al., 1999; Washburn et al., 2001). Peptide mixture was pressure-loaded onto a 250-μm i.d. fused silica capillary column packed with 2.5 cm Partisphere strong cation exchanger (Whatman, Clifton, N.J.) and 2.5 cm 5-μm Aqua C18 material (Phenomenex, Ventura, Calif.). The column was washed for 30 min with buffer containing 95% water, 5% acetonitrile (ACN), and 0.1% formic acid. After desalting, it was attached to a 100-μm i.d. capillary with a 5-μm pulled tip packed with 12 cm 5-μm Aqua C18 material, and the entire column was placed inline with an Agilent 1100 quaternary HPLC (Agilent, Palo Alto, Calif.). The sample was analyzed using a fully automated 12-step separation procedure. The buffer solutions used for the chromatography were 5% ACN/0.1% FA (buffer A), 80% ACN/0.1% FA (buffer B), and 500 mM ammonium acetate/0.1% FA (buffer C). The first step consisted of a 100 min gradient from 0 to 100% buffer B. Steps 2-11 had the following profile: 3 min of 100% buffer A, 3 min of X % buffer C, a 10 min gradient from 0 to 15% buffer B, and a 97 min gradient from 15 to 55% buffer B. The 3 min buffer C percentages (X) were 5, 10, 20, 30, 40, 50, 60, 70, 80 and 90%, respectively. In the final step, the gradient contained: 3 min of 100% buffer A, 10 min of 100% buffer C, a 10 min gradient from 0 to 15% buffer B, and a 107 min gradient from 15 to 100% buffer B. As peptides were being eluted from the microcapillary column, they were electrosprayed directly into a linear LTQ ion trap mass spectrometer (ThermoFinnigan, San Jose, Calif.) with the application of a 2.4 kV spray voltage. A cycle of one full scan mass spectrum (400-1400 m/z) followed by 5 data-dependent MS/MS spectra, at a 35% normalized collision energy and with dynamic exclusion enabled, was repeated continuously throughout each step of the multidimensional separation.

Acquired tandem mass spectra were searched against the European Bioinformatics Institute International Protein Index human protein database (version 3.30, released on Jun. 28, 2007). In order to calculate confidence levels and false positive rates, a decoy database containing the reverse sequences was appended to the target database, and the SEQUEST (Eng et al., 1994) algorithm was used to find the best matching sequences from the combined database. SEQUEST results were assembled and filtered by DTASelect (Tabb et al., 2002). At least two peptides per protein and a false positive rate of less than 1% at the protein level were required.

Estimation of protein abundance based on spectra count was used as the relative quantification method (Liu et al., 2004) which has been widely applied (Cao et al., 2008; Liao et al., 2007; Rikova et al., 2007). Spectra counts from the three replicates of each sample were merged to average the run to run variation. Although the total number of spectra was similar between any two samples, a normalization factor (F=Total number of spectra in control sample/Total number of spectra in treated sample) was applied, that is, the spectra count ratio of the treated sample versus the control sample multiplied by the normalization factor gives the normalized ratio. If a protein is detected in both untreated and treated samples, proteins with expression level changes were filtered according to the following criteria: (1) if the same protein was identified in both samples with spectra counts greater than 10, normalized spectra count ratios of 2 or above were considered as increased, likewise, 0.5 or less as decreased; (2) if the same protein was identified in both samples with a spectra count from either of them less than 10 but the difference between the two was great than 10, normalized spectra count ratios of 2.5 or above were considered as increased, likewise, 0.4 or less as decreased. If a protein was identified in only one sample, a spectra count of greater than 20 was used to consider a significant change; a preliminary analysis of this category showed that treatment of L444P GC fibroblast with MG-132 (0.8 μM) for 3 d upregulated 83 proteins and down regulated 85 proteins, while treatment of L444P GC fibroblast with celastrol (0.8 μM) for 3 d upregulated 106 proteins and down regulated 87 proteins, amongst the 1000 proteins detected in this category, indicating that the PR treatment modestly affect the proteome globally along with the data shown in FIG. 2A, where a protein is detected in both untreated and treated samples.

Quantitative RT-PCR. The cells were incubated with drugs at 37° C. for the indicated amount of time. Total RNA was extracted from the cells using RNeasy Mini Kit (Qiagen #74104). cDNA was synthesized from 500 ng of total RNA using QuantiTect Reverse Transcription Kit (Qiagen #205311). Quantitative PCR reactions were performed using cDNA, QuantiTect SYBR Green PCR Kit (Qiagen #204143) and corresponding primers in the ABI PRISM 7900 system (Applied Biosystems). The forward and reverse primers for Hsp40, Hsp70, Hsp90, Hsp27, αB-crystallin (CRYAB), BiP, GRP94, calnexin (CNX), calreticulin (CRT), Xbp-1, and CHOP, and an endogenous housekeeping gene GAPDH are listed in Table 2. Samples were heated for 15 min at 95° C. and amplified in 45 cycles of 15 s at 94° C., 30 s at 57° C., and 30 s at 72° C. Analysis was done using SDS2.1 software (Applied Biosystems). Threshold cycle (C_{T}) was extracted from the PCR amplification plot. The ΔC_{T} value was used to describe the difference between the C_{T} of a target gene and the C_{T} of the housekeeping gene: ΔC_{T}=C_{T} (target gene)−C_{T} (housekeeping gene). The relative mRNA expression level of a target gene of drug-treated cells was normalized to that of untreated cells: Relative mRNA expression level=2exp[−(ΔC_{T} (treated cells)−ΔC_{T}(untreated cells))]. Each data point was evaluated in triplicate, and measured three times.

TABLE 2

| | GenBank | | |
|---|---|---|---|
| | Accession | | |
| Gene | code | Forward Primer | Reverse Primer |
| GAPDH | NM_002046 | 5′-GTC GGA GTC AAC GGA TT-3′ | 5′-AAG CTT CCC GTT CTC AG-3′ |
| Hsp40 | NM_006145 | 5′-CGC CGA GGA GAA GTT C-3′ | 5′-CAT CAA TGT CCA TGC CTT-3′ |
| Hsp70 | NM_005345 | 5′-GGA GGC GGA GAA GTA CA-3′ | 5′-GCT GAT GAT GGG GTT ACA-3′ |
| Hsp90 | NM_005348 | 5′-GAT AAA CCC TGA CCA TTC C-3′ | 5′-AAG ACA GGA GCC CAG TTT CAT |
| | | | AAA-3′ |
| Hsp27 | X54079 | 5′-AAG TTT CCT CCT CCC TGT CC- | 5′-CGG GCT AAG GCT TTA CTT GG- |
| | | 3′ | 3′ |
| CRYAB | NM_001885 | 5′-CAC CCA GCT GGT TTG ACA CT- | 5′-TGA CAG AGA ACC TGT CCT TCT- |
| | | 3′ | 3′ |
| BiP | NM_005347 | 5′-GCC TGT ATT TCT AGA CCT GCC- | 5′-TTC ATC TTG CCA GCC AGT TG- |
| | | 3′ | 3′ |
| GRP94 | NM_003299 | 5′-GGC CAG TTT GGT GTC GGT TT- | 5′-CGT TCC CCG TCC TAG AGT GTT- |
| | | 3′ | 3′ |
| CNX | NM_001746 | 5′-GCG TTG TGG GGC AGA TGA T-3′ | 5′-CCG GTT GAG GTG CAT CAG T-3′ |
| CRT | NM_004343 | 5′-AAG TTC TAC GGT GAC GAG GAG- | 5′-GTC GAT GTT CTG CTC ATG TTT |
| | | 3′ | C-3′ |
| CHOP | NM_004083 | 5′-ACC AAG GGA GAA CCA GGA AAC | 5′-TCA CCA TTC GGT CAA TCA GAG |
| | | G-3′ | C-3′ |
| Xbp-1 | NM_005080 | 5′-TTA CGA GAG AAA ACT CAT GGC- | 5′-GGG TCC AAG TTG TCC AGA ATG |
| | | 3′ | C-3′ |

RT-PCR analysis of Xbp-1 splicing. cDNA was synthesized as in quantitative RT-PCR. PCR reactions were performed using cDNA, Taq DNA polymerase (Roche) and corresponding primers listed in Table 2. Samples were heated for 5 min at 95° C., amplified in 30 cycles of 60 s at 95° C., 30 s at 58° C., and 30 s at 72° C., and 5 min at 72° C. PCR products were subjected to a 2.5% agarose gel. Unspliced Xbp-1 yielded a 289 bp amplicon, and spliced Xbp-1 yielded a 263 bp amplicon. The experiments were performed three times and similar results were obtained.

### Example 13

### The L-Type Ca²⁺ Channel Blockers Diltiazem and Verapamil Enhance Lysosomal GC Activity in Gaucher Patient-Derived Fibroblast Cell Lines

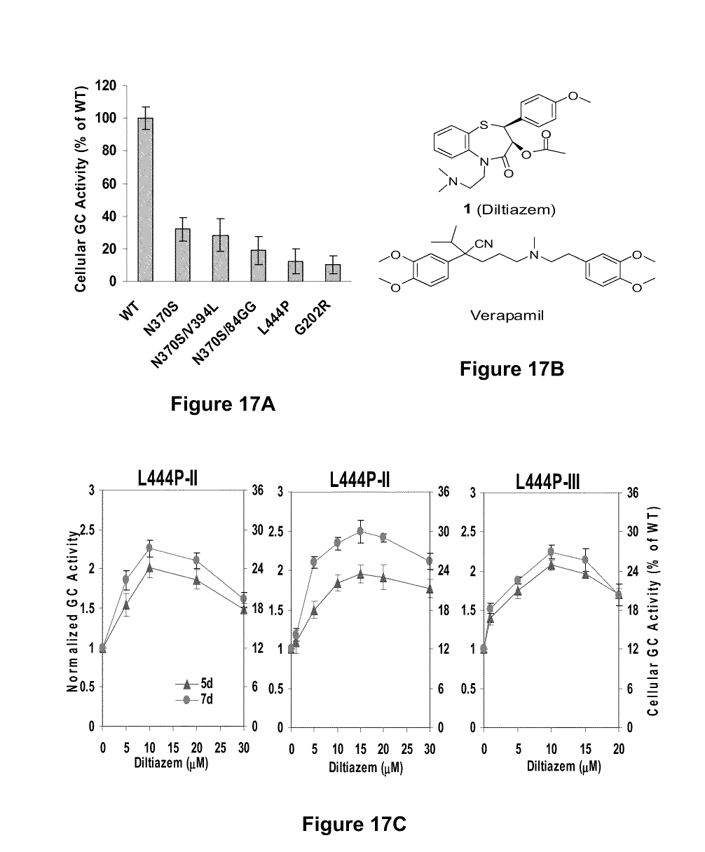

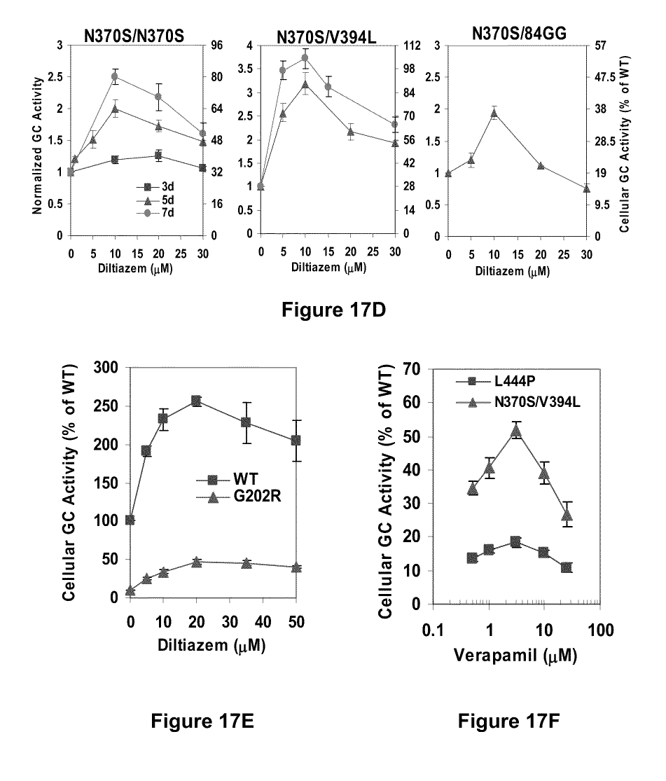

Several Type I, II, and III Gaucher fibroblast cell lines were evaluated to discern the relative activity of the mutant GC harbored by equal numbers of cells reflected by equal amount of total protein in the cell lysate. The residual enzymatic activities of the GC variants were measured using the lysed cell activity assay, FIG. 17A. Under the assay conditions used, L444P GC exhibits 12% of the activity of wild type (WT) GC; N370S GC, 32%; N370S/V394L GC, 28%; N370S/84GG GC, 19%; and G202R GC, 10% of the activity of WT GC. The exact enzyme activity measured is highly dependent on the assay conditions. Sawkar et al. Chem Biol 12: 1235-1244, 2005. The enzyme activities displayed were normalized to the activity of untreated cells of the same type and expressed as fold changes. In addition, mutant GC activity before and after treatment was also expressed as the percentage of WT GC activity to calibrate the reader.

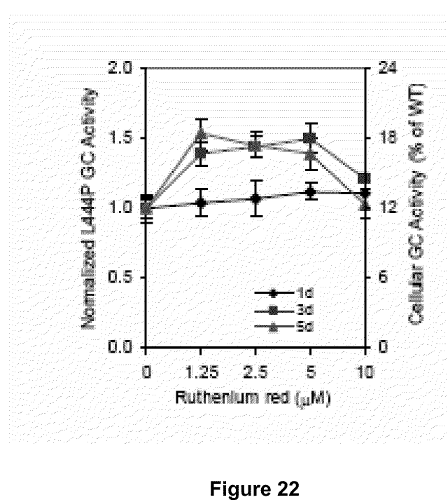

Gaucher patient-derived fibroblasts harboring the L444P GC mutation grown at 30° C., instead of at 37° C., exhibit enhanced folding, trafficking, lysosomal localization and activity of L444P GC. Sawkar et al., ACS Chem Biol 1: 235-251, 2006. Because a change in temperature alters both the physical chemistry of the L444P protein and the cellular protein homeostasis machinery, the hypothesis that cells were readjusting their protein homeostasis capacity through their thermosensitive TRP channels was tested. Fifteen thermosensitive TRP channel modulators (capsaicin, resiniferatoxin, piperine, olvanil, anandamide, 2-APB, camphor, 4α-PDD, menthol, eucalyptol, icilin, cinnamaldehyde, allylisothiocyanate, capsazepine, and ruthenium red) were administered to homozygous L444P GC patient-derived fibroblasts. Voets et al., Nat Chem Biol 1: 85-92, 2005; Dhaka et al., Annu Rev Neurosci 29: 135-161, 2006. Enhanced L444P GC folding and trafficking could be inferred from an increased lysosomal L444P GC activity measured using the intact cell GC enzyme assay. Only ruthenium red notably increased L444P GC activity after a 5-day incubation period (FIG. 22). Since the only TRP channel modulator that enhanced lysosomal L444P GC activity was also a non-specific Ca²⁺ channel blocker, the observed increase might be a consequence of a lowered intracellular Ca²⁺ ion concentration and thus it seemed unlikely that TRP channel modulation is the means by which temperature regulates the intracellular protein homeostasis capacity.

FIG. 22 shows the influence of ruthenium red on L444P glucocerebrosidase (GC) activity in Gaucher patient-derived fibroblasts after culturing for 1 day (black line), 3 days (pink line), and 5 days (green line). The GC activity of treated cells was normalized against that of untreated L444P GC cells (left y axis) and expressed as the percentage of WT GC activity (right y axis).

The hypothesis that ruthenium red was lowering the intracellular Ca²⁺ ion concentration and enhancing GC folding fidelity by that means was scrutinized experimentally by blocking calcium entry into the cell through its calcium channels, including voltage-gated calcium channels (VGCCs) and ionotropic glutamate receptors. Berridge et al., Nat Rev Mol Cell Biol 4: 517-529, 2003; Elmslie, J Neurosci Res 75: 733-741, 2004; Mayer et al., Annu Rev Physiol 66: 161-181, 2004. Lysosomal L444P GC activity was evaluated after application of nine representative VGCC blockers, namely diltiazem, verapamil, nifedipine, nimodipine, loperamide, mibefradil, ethosuximide, flunarizine, and bepridil, five representative ionotropic glutamate receptor inhibitors, namely CGP 39551, 5,7-dichlorokynurenic acid, DNQX, Evans blue, and felbamate, and other calcium channel blockers, such as amiodarone, cinnarizine, and SKF 96365, to L444P GC patient-derived fibroblasts. The intact cell GC activity assay revealed that only diltiazem and verapamil (chemical structures shown in FIG. 17B) increased L444P GC activity significantly. Diltiazem increased L444P GC activity a maximum of 2.0-fold (to ≈24% of normal cellular WT GC activity) after a 5-day incubation period, and 2.3-fold after a 7-day incubation period (FIG. 17C, left panel) at a 10 μM concentration (all concentrations mentioned are cell culture concentrations unless otherwise stated), implying an increased lysosomal L444P GC concentration. The temporal dependence of the diltiazem-mediated cellular L444P GC activity increase is very similar to the time dependence of the cellular N370S GC activity increase observed upon pharmacological chaperone treatment [Sawkar et al. Chem Biol 12: 1235-1244, 2005]. The slow gain in activity of the GC variants is partially a result of the slower folding and trafficking of the variants as revealed by prior pulse chase experiments and likely for other reasons, for example, the apparent requirement for the transcription and translation of selected chaperones (vide infra). Steet et al., Proc Natl Acad Sci USA 103: 13813-13818, 2006; Schmitz et al., Int J Biochem Cell Biol 37:2310-2320, 2005. This slow mutant GC activity increase upon pharmacological chaperone or diltiazem administration is also observed upon pharmacological chaperone administration in different lysosomal storage diseases such as Fabry disease, Tay-Sachs disease, and Pompe disease. Fan et al., Nat Med 5: 112-115, 1999; Parenti et al., Mol Ther 15: 508-514, 2007; Tropak et al., J Biol Chem 279: 13478-13487, 2004.

To confirm that the effect of diltiazem was not restricted to one L444P GC patient-derived cell line, two additional patient-derived homozygous L444P GC fibroblast cell lines were treated with diltiazem (15 μM). In a type II cell line, diltiazem increased the GC activity up to 2.0-fold after an incubation period of 5 days and up to 2.5-fold after 7 days of treatment (FIG. 17C, middle panel). Diltiazem (10 μM) treatment of a type III Gaucher patient-derived cell line increased L444P GC activity to a maximum of 2.1-fold after a 5-day incubation period and up to 2.3-fold after a 7-day incubation period (FIG. 17C, right panel). Lysosomal L444P GC activity was improved in all the neuropathic fibroblast cell lines evaluated.

If diltiazem regulates mutant GC homeostasis by a general mechanism, such as a cellular chaperone mediated mechanism, and not by binding induced pharmacological chaperoning, it should also be able to enhance the folding, trafficking and activity of other misfolding prone GC variants in homozygous and compound heterozygous Gaucher patient-derived cell lines. Diltiazem (10 μM) increased N370S GC activity up to 2.0-fold (to ≈64% of untreated WT GC activity) after a 5-day incubation period and up to 2.5-fold after a 7-day incubation period in N370S GC fibroblasts from a homozygote (FIG. 17D, left panel), analogous to the best results obtained with optimized pharmacological chaperones. Yu et al., I J Med Chem 50: 94-100, 2007. In the case of a compound heterozygous N370S/V394L GC cell line, diltiazem (10 μM) increased the GC activity up to 3.2-fold (to ≈89% of cellular WT GC activity) after an incubation period of 5 days and up to 3.7-fold after 7 days of treatment (FIG. 17D, middle panel). In the analogous N370S/84GG GC cell line, diltiazem (10 μM) increased the GC activity up to 1.9-fold (to ≈36% of cellular WT GC activity) after a 5-day treatment (FIG. 17D, right panel). Diltiazem (20 μM) increased G202R GC activity up to 4.6-fold (to ≈46% of cellular WT GC activity) after a 5-day incubation period (FIG. 17E, green line), demonstrating the generality of diltiazem to regulate GC protein homeostasis. Notably, diltiazem (20 μM) increases WT GC activity up to 2.6-fold after 5 days of treatment (FIG. 17E, pink line), suggesting that the folding and trafficking of WT GC is inefficient, as is the case for other proteins such as G-protein-coupled receptors. Ulloa-Aguirre et al., ACS Chem Biol 1: 631-638, 2006] and ion channels [Green et al., Trends Neurosci 18: 280-287, 1995.

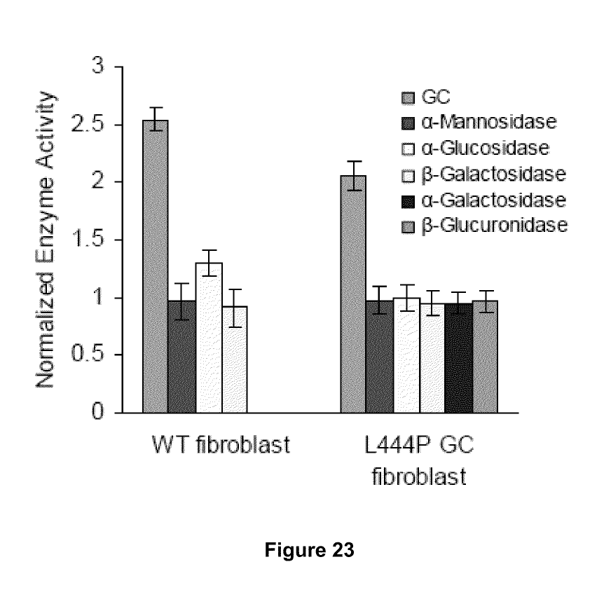

The influence of diltiazem on the cellular activity of other WT lysosomal hydrolases, namely α-mannosidase, α-glucosidase, β-galactosidase, α-galactosidase, and β-glucuronidase was evaluated. WT fibroblasts and L444P GC fibroblasts were incubated with diltiazem (10 μM) for 5 days before the analysis (FIG. 23). While diltiazem treatment increased GC activity, it did not significantly increase the activity of other WT lysosomal enzymes, implying that the folding and trafficking of these enzymes is near optimal.

FIG. 23 shows the influence of diltiazem on the activity of lysosomal enzymes. After incubation with 10 μM diltiazem for 5 days, WT fibroblasts were assayed for the activities of GC, α-mannosidase, α-glucosidase, and β-galactosidase using intact cell enzyme activity assay, and L444P GC cells were assayed for the activities of GC, α-mannosidase, α-glucosidase, β-galactosidase, α-galactosidase, and β-glucuronidase using lysed cell enzyme activity assay. The enzyme activity of treated cells was normalized against that of untreated cells of the same type.

A second L-type Ca²⁺ channel blocker, verapamil (3 μM), increased L444P GC activity up to 1.5-fold (to 18% of cellular WT GC activity) and N370S/V394L GC activity up to 1.9-fold (to 53% of cellular WT GC activity) after a 7-day incubation period (FIG. 17F; lysed cell activity assay). That both diltiazem and verapamil, Ca²⁺ channel blockers with distinct chemical structures (FIG. 17B), enhance cellular mutant GC folding, trafficking and activity, supports the hypothesis that altering intracellular Ca²⁺ homeostasis influences lysosomal enzyme homeostasis.

FIG. 17 shows influence of small molecules on glucocerebrosidase (GC) variant activity in Gaucher patient-derived fibroblasts. (A) Residual activities of GC variants using the lysed cell GC activity assay, employing equal numbers of cells as ascertained from the equal total protein content of the lysate. Residual activities of N370S, N370S/V394L, N370S/84GG, L444P, and G202R GC are shown as the percentage of WT GC activity (numbers above each column), respectively. (B) Chemical structures of diltiazem (compound 1) and verapamil. (C) The influence of diltiazem (1) on L444P GC activity in three distinct homozygous L444P GC patient derived cell lines: L444P GC fibroblasts from a type II patient (left panel), another type II patient (middle panel), and a type III patient (right panel). These cell lines were cultured with diltiazem for 5 days (green line) and 7 days (orange line), respectively. The GC activity of treated cells was normalized against that of untreated cells of the same type (left y axis) and expressed as the percentage of WT GC activity (right y axis). (D) The influence of diltiazem on N370S GC activity in homozygous N370S/N370S GC fibroblasts (left panel), heterozygous N370S/V394L fibroblasts (middle panel), and heterozygous N370S/84GG fibroblasts (right panel). These cell lines were cultured with diltiazem for 3 days (pink line), 5 days (green line), and 7 days (orange line), respectively. The GC activity of treated cells was normalized against that of untreated cells of the same type (left y axis) and expressed as the percentage of WT GC activity (right y axis). (E) The influence of diltiazem on WT and G202R GC activity. WT and G202R GC cell lines were cultured with diltiazem for 5 days, respectively. The GC activity was expressed as the percentage of WT GC activity. (F) The influence of verapamil on L444P and N370S/V394L GC activity. These cell lines were treated with verapamil for 7 days, respectively. The GC activity was expressed as the percentage of WT GC activity.

### Example 14

GC Exhibits a Dose-Dependent Concentration Increase Upon Diltiazem Treatment.

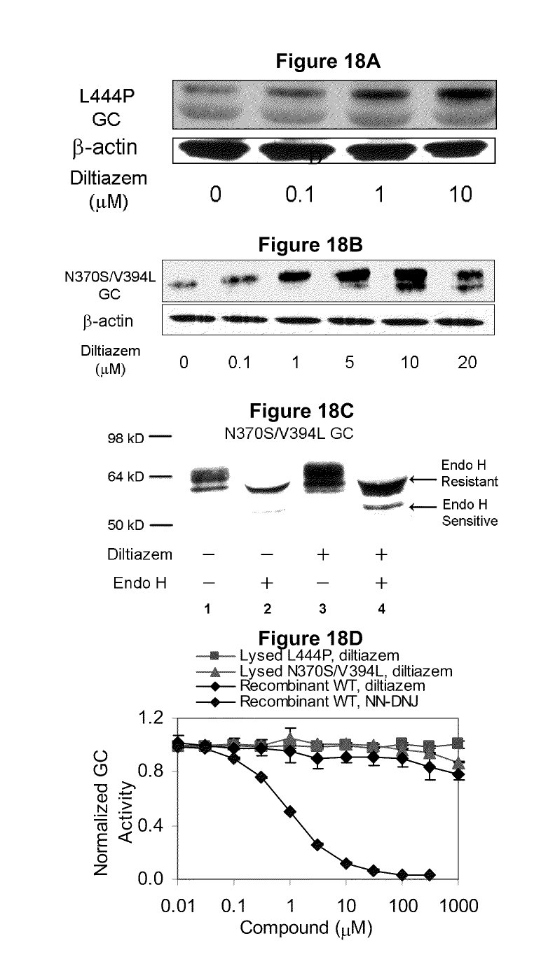

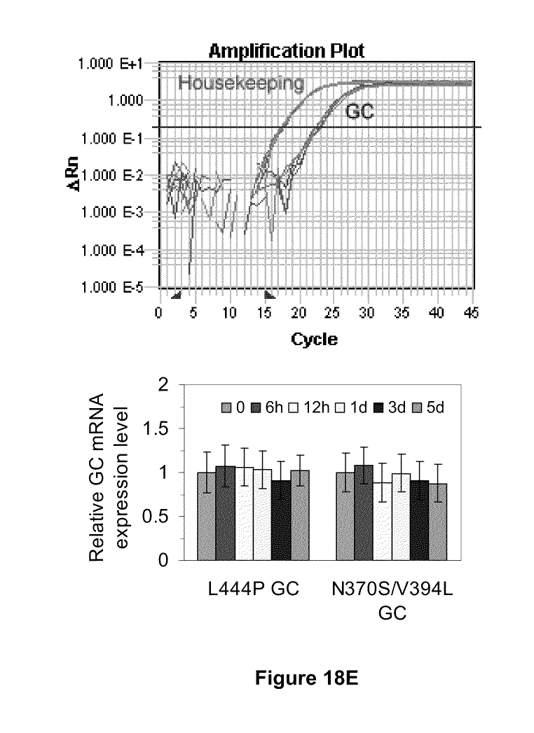

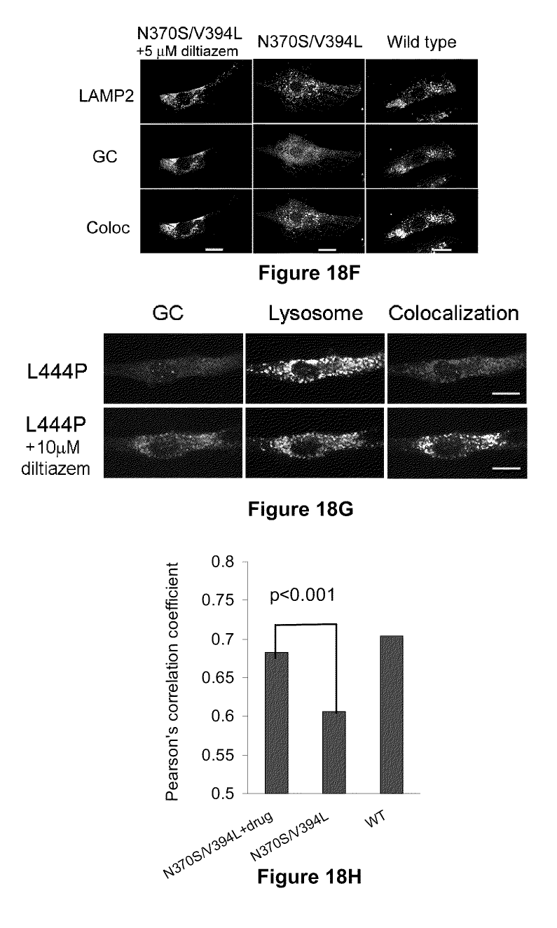

Western blot analysis reveals that L444P GC concentrations were elevated in a dose-dependent manner in type II Gaucher fibroblasts after a 7-day treatment with diltiazem (FIG. 18A). β-actin served to ensure that equal amounts of total protein were loaded in each lane. The GC band intensity increases with the concentration of added diltiazem (0, 0.1, 1 and 10 μM), consistent with the observed dose-dependent increase in GC enzymatic activity (FIG. 17C, left panel).

The patient-derived N370S/V394L GC cell line was also cultured with diltiazem (0.1-10 μM) for 7 days, revealing an analogous dose-dependent increase in GC band intensity (FIG. 18B), consistent with the concentration dependent GC activity increase (FIG. 17D, middle panel). An endo-H digestion was performed on treated and untreated N370S/V394L GC cells to demonstrate that the mature lysosomal glycoform of GC, associated with proper lysosomal trafficking, was being produced. After 7 days of cell culturing, equal numbers of diltiazem treated (10 μM) and untreated cells (reflected by equal amounts of total protein) were subjected to endo-H treatment or buffer only treatment before separation on a 10% SDS-PAGE gel and detection of GC by western blot analysis (FIG. 18C). The upper bands in lanes 2 and 4 corresponding to the endo-H resistant, mature lysosomal GC glycoform increase upon diltiazem treatment [10], demonstrating that substantially more properly folded GC protein was trafficked out of the ER and to the lysosome after diltiazem treatment. The lower bands in lanes 2 and 4 correspond to the endo-H sensitive, ER GC glycoform.

### Example 15

### Ruling Out a Pharmacological Chaperoning Mechanism and a Direct Lysosomal GC Activation Mechanism

All of the GC pharmacological chaperones discovered to date are active-site directed stabilizers and are thus enzyme inhibitors; therefore we evaluated whether diltiazem binds to the active site and inhibits GC. Lysed L444P fibroblasts and lysed N370S/V394L cells were incubated with diltiazem (0.01 to 1000 μM) and assayed. No significant GC inhibition was observed in either case (FIG. 18D, pink and green lines). Cerezyme, a recombinant version of WT GC, was also incubated with diltiazem (0.01 to 1000 μM), revealing lack of inhibition (FIG. 18D, black line). As a positive control, an established GC pharmacological chaperone, N-(n-nonyl)deoxynojirimycin (NN-DNJ), exhibiting a half maximal inhibitory concentration (IC₅₀) value of 1.08 μM toward Cerezyme (FIG. 18D, blue line), exhibited inhibition. Sawkar et al., Proc Natl Acad Sci USA 99: 15428-15433, 2002. Collectively, these results demonstrate that diltiazem does not bind to the active site of GC ex vivo and is unlikely to function as a pharmacological chaperone.

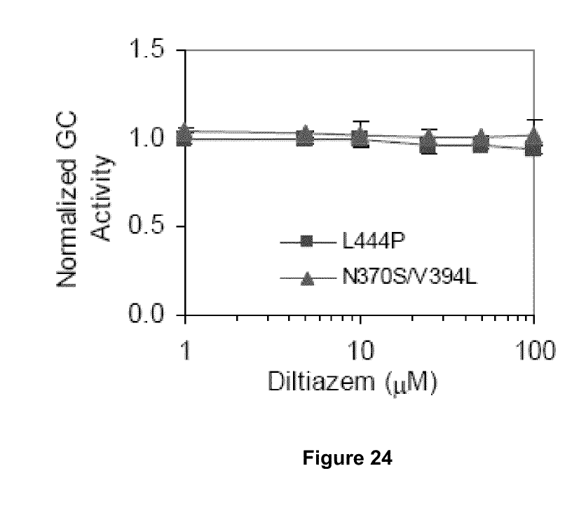

To evaluate whether diltiazem could directly activate the existing lysosomal GC pool, L444P and N370S/V394L GC fibroblasts were incubated with diltiazem (1 μM to 100 μM) for 1 h and the GC activity was measured using the intact cell assay. No activity increase was observed (FIG. 24), demonstrating that the GC activity increase could not be achieved on this short time scale, a result inconsistent with direct diltiazem-induced saposin mediated activation of GC. A relatively long incubation period (5 days) is required for diltiazem to maximally increase intralysosomal L444P GC activity 2.0-fold (FIG. 17C, left panel) and N370S/V394L GC activity 3.2-fold (FIG. 17D, middle panel), consistent with previous findings showing nearly identical rates of GC activity increases mediated by diltiazem treatment and pharmacological chaperone treatment. Sawkar et al. Chem Biol 12: 1235-1244, 2005.

FIG. 24 shows L444P and N370S/V394L GC cells were incubated with diltiazem for 1 hour, and their GC activities were evaluated using the intact cell GC activity assay. The GC activity of treated cells was normalized against that of untreated cells of the same type.

### Example 16

### Diltiazem does not Influence GC Transcription

Quantitative reverse transcription-polymerase chain reaction (RT-PCR) analysis was performed on L444P GC fibroblasts incubated without and with 10 μM diltiazem for 6 h, 12 h, 1 day, 3 days and 5 days. Real-time PCR reactions were performed on total DNA reverse-transcribed from total RNA samples, which were extracted from L444P GC harboring cells. The PCR amplification plot is shown in FIG. 18E, left panel. ΔC_{T} is defined as the difference between the threshold cycle (C_{T}) value of the GC gene and the C_{T} value of a housekeeping gene. The relative GC mRNA expression level was normalized to that of untreated GC cells, calculated from corresponding ACT values (see materials and methods). No significant differences for the GC mRNA expression levels were observed when comparing untreated and diltiazem-treated L444P GC cells (FIG. 18E, right panel, left entries) demonstrating that diltiazem does not influence GC transcription in L444P GC cells. Strictly analogous results were obtained for diltiazem-treated N370S/V394L GC cells (FIG. 18E, right panel, right entries).

### Example 17

### Diltiazem Enhances Proper GC Folding and Trafficking

Cellular trafficking of L444P and N370S/V394L GC appears to be reduced because of ERAD outcompeting folding and trafficking. Ron et al., Hum Mol Genet. 14: 2387-2398, 2005. Fluorescence microscopy was previously utilized to demonstrate that active-site directed pharmacological chaperones enhance the folding and trafficking of G202R GC to the lysosome [10]. Strictly analogous immunofluorescence microscopy methods were utilized to demonstrate that Ca²⁺ channel blockers increase L444P and N370S/V394L GC trafficking to the lysosome. L444P GC harboring fibroblasts were cultured without or with 10 μM diltiazem for 14 days prior to plating for microscopy. WT GC fibroblasts were also studied analogously as a positive control. A properly folded and trafficked GC protein will colocalize with the lysosomal marker LAMP2 [10]. WT GC distributed in a punctate manner, and colocalized with LAMP2 (FIG. 18F, column 3, row 3, GC in green, LAMP2 in red, and overlap artificially colored white). This color scheme is used only for the colocalization row; for single staining experiments (the first two rows), the fluorescence images are artificially colored white to improve contrast. While the L444P GC variant was not visible without diltiazem treatment, due to extensive ERAD, it was easily detected and was distributed in a punctate manner after diltiazem treatment (FIG. 18G, column 1). L444P GC colocalized with LAMP2 after diltiazem treatment (FIG. 18G, column 3, GC in green, LAMP2 in red, and overlap artificially colored white), indicating increased lysosomal trafficking, consistent with the increase in cellular GC concentrations (FIG. 18A) and the increase in enzymatic activity (FIG. 17C).

Previous experiments demonstrate that the N370S GC distribution is partially lysosomal [10]. To determine whether the increase in properly glycosylated N370S/V394L GC protein observed in response to diltiazem treatment (FIG. 18C) resulted in an increase in proper trafficking to the lysosome, quantitative immunofluorescence microscopy was performed. N370S/V394L GC fibroblasts were incubated without and with 5 μM diltiazem for 7 days, prior to plating for microscopy. WT GC fibroblasts serve as a control. While measurable N370S/V394L GC colocalizes with the lysosome, there is substantially less N370S/V394L GC in the lysosome in comparison to WT GC (FIG. 18F, compare column 2 with column 3), consistent with significant ERAD. Diltiazem treatment notably enhanced N370S/V394L GC trafficking to the lysosome (FIG. 18F, compare column 1 with column 2), consistent with its ability to increase the concentration of the mature GC glycoform, FIG. 18B/C. Quantification of the colocalization between the GC protein and the lysosomal marker utilizing twenty random microscope fields for each sample was accomplished using Pearson's correlation coefficient (PCC). WT GC, untreated N370S/V394L GC, and diltiazem-treated N370S/V394L GC fibroblasts have PCC values of 0.70±0.06, 0.60±0.05, and 0.68±0.05, respectively (FIG. 18H). The difference between the PCC values of untreated and diltiazem-treated N370S/V394L GC cells is significant (p<0.001, n=20), demonstrating that diltiazem increased trafficking of N370S/V394L GC to the lysosome, nearly to WT levels.

FIG. 18 shows effect of diltiazem on L444P and N370S/V394L GC folding and trafficking. (A) Western blot analysis of untreated and diltiazem-treated L444P GC cells. L444P GC cells were cultured without or with diltiazem at varying concentrations for 7 days before the cells were lysed for SDS-PAGE and western blot analysis. GC was detected using mouse anti-GC antibody 2E2. β-actin served as a loading control. (B) Western blot of untreated and diltiazem-treated N370S/V394L GC cells. N370S/V394L GC cells were incubated with variable diltiazem concentrations for 7 days before the cells were lysed for SDS-PAGE and western blot analysis using mouse anti-GC antibody 8E4. (C) The endo-H sensitivity of untreated and diltiazem-treated N370S/V394L GC cells. N370S/V394L GC cells were incubated without and with 10 μM diltiazem for 7 days before the cells were lysed for endo-H digestion, SDS-PAGE and western blot analysis using mouse anti-GC antibody 8E4. (D) L444P and N370S/V394L GC cells were lysed and equal amount of total cell protein was incubated with diltiazem and their GC activities were evaluated using the lysed cell GC activity assay. Cerezyme, a recombinant WT GC protein, was also tested for its GC activity after treatment with diltiazem (black line) or a pharmacological chaperone NN-DNJ, a known inhibitor (blue line). (E) Quantitative RT-PCR on untreated and diltiazem-treated L444P and N370S/V394L GC cells. L444P and N370S/V394L GC cells were incubated with 10 μM diltiazem for 6 h, 12 h, 1 d, 3 d and 5 d, respectively. The figure on the left is the representative amplification plot for the quantitative PCR cycles using L444P GC cells; the figure on the right shows the relative GC mRNA expression level for diltiazem-treated L444P (left entries) and N370S/V394L GC cells (right entries), respectively, which is normalized to that of untreated cells. (F) Immunofluorescence colocalization analysis of GC in N370S/V394L GC and WT fibroblasts. N370S/V394L GC cells were incubated with 5 μM diltiazem for 7 days (column 1) or cultured without drug (column 2). Untreated WT cells were observed as a positive control (column 3). GC was visualized using mouse anti-GC antibody 16B3 (row 2) and rabbit anti-LAMP2 antibody was applied as a lysosomal marker (row 1). In row 3, the colocalization of GC (green) and LAMP2 (red) was shown in white. Bar=10 μm. (G) Immunofluorescence colocalization analysis of GC in L444P GC cells. L444P GC cells were incubated with 10 μM diltiazem for 14 days (bottom row) or untreated (top row). GC visualization was accomplished using the mouse anti-GC antibody 8E4 (column 1); rabbit anti-LAMP2 antibody was used as a lysosomal marker (column 2). In column 3, the colocalization of GC (green) and LAMP2 (red) was artificially colored white. Bar=20 μm. (H) Quantification of the colocalization between the GC protein and the lysosomal marker using Pearson's correlation coefficient. Experimental conditions were stated in FIG. 18F.

### Example 18

### Extracellular Ca²⁺ Concentration Influences Intracellular Folding Capacity

Diltiazem and verapamil are both potent L-type voltage-gated calcium channel blockers that inhibit Ca²⁺ entry from the extracellular medium into the cell and thus alter calcium homeostasis in the cell. Triggle, Curr Pharm Design 12: 443-457, 2006. The cytoplasmic free Ca²⁺ ion concentration (ca. 100 nM) is much lower than the extracellular Ca ion concentration (ca. 2 mM) at steady state in a normal cellular environment. We explored whether manipulation of the extracellular Ca²⁺ concentration for prolonged periods could alter intracellular GC folding, trafficking and activity.

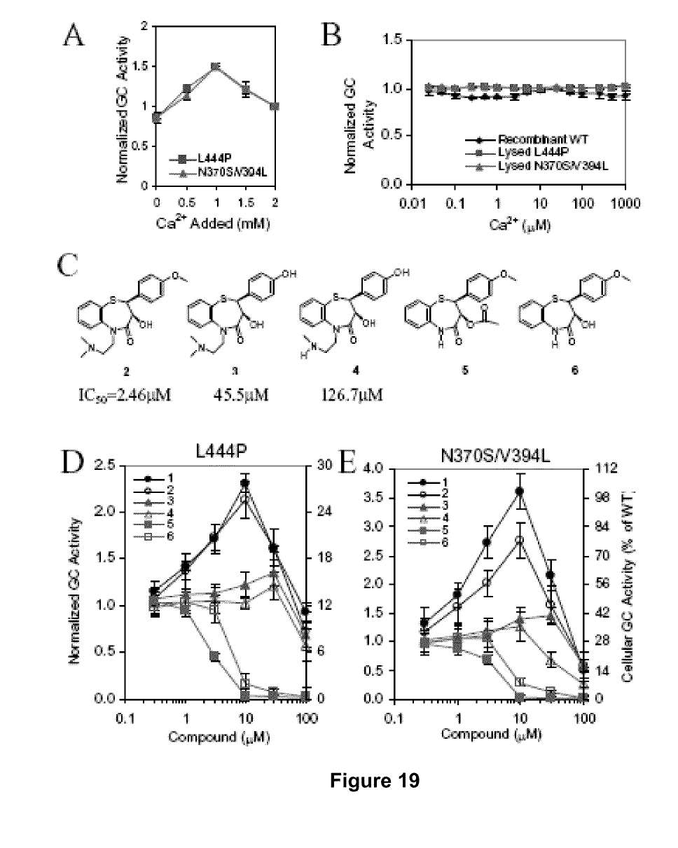

Different Ca²⁺ ion concentrations (0, 0.5, 1, 1.5, and 2 mM CaCl₂) added to Ca²-free cell culture media (supplemented with FBS) were applied to L444P GC cells for 10 days and to N370S/V394 GC cells for 7 days. The GC activity was then evaluated using the lysed cell GC activity assay. GC activity was normalized to that observed with 2 mM Ca²⁺ added in the media, similar to the concentration used in other experiments reported herein. The maximum GC activity increase (1.5-fold) was achieved when 1 mM Ca²⁺ was added to the media of L444P and N370S/V394L GC cells, demonstrating the important influence of extracellular Ca ion concentration on GC folding and trafficking (FIG. 19A).

Whether Ca²⁺ ions can interact directly with the GC protein was explored. Lysed L444P and N370S/V394L GC cells were incubated with variable Ca²⁺ ion concentrations (25 nM to 2 mM) and assayed using the lysed cell GC activity assay, indicating no significant changes in GC activity (FIG. 19B). Cerezyme, a recombinant version of WT GC, was evaluated analogously, revealing unaltered activity (FIG. 19B, black line). These results demonstrate that Ca²⁺ ions do not directly activate or inhibit the GC protein ex vivo.

### Example 19

### GC Activity Enhancement Correlates with Ca²⁺ Ion Channel Blocker Activity

To further examine the hypothesis that diltiazem enhances GC activity through its Ca²⁺ ion channel blocker activity, five diltiazem analogs exhibiting a range of potencies were procured (FIG. 19C). The previously reported Ca²⁺ channel blocker IC₅₀ values are: 1 (diltiazem, IC₅₀=0.98 μM)>2 (2.46 μM)>3 (45.5 μM)>4 (126.7 μM). Li et al., J Med Chem 35: 3246-3253, 1992. Analogs 5 and 6 should not block Ca²⁺ ion channel activity because they lack a key basic amino nitrogen pharmacophore linked to N5 in the benzothiazepine ring scaffold, according to a reported structure-activity relationship (SAR) study on benzazepinone and a quantitative SAR study on diltiazem. Kimball et al., J Med Chem 35: 780-793, 1992; Kettmann et al., Quant Struct-Act Relat 17: 91-101, 1998.

L444P GC fibroblasts were cultured with compounds 1-6 (0.3 to 100 μM) for 7 days and dose-response curves were recorded using the intact cell GC activity assay (FIG. 19D). Compounds 1 (IC₅₀=0.98 μM) and 2 (IC₅₀=2.46 μM) are potent Ca²⁺ channel antagonists, and exhibit notable L444P lysosomal GC activity increases to a maximum of 2.3-fold for 1 and 2.1-fold for 2 at 10 μM (FIG. 19D, black lines). Compounds 3 (IC₅₀=45.5 μM) and 4 (IC₅₀=126.7 μM) are both weak Ca²⁺ channel antagonists, and weak L444P GC activity enhancers. Notably, only at much higher concentrations (30 μM) do these low potency analogs exhibit a maximum increase in L444P GC activity of 1.3-fold for 3 and 1.2-fold for 4 (FIG. 19D, pink lines). Compounds 5 and 6 are not Ca²⁺ channel antagonists, and, as such, these closely related analogs do not increase L444P GC activity (FIG. 19D, green lines). These data demonstrate that the more potent the Ca²⁺ ion channel blocker, the higher the lysosomal GC activity enhancement observed. Diltiazem and its analogs were also analogously tested in N370S/V394L fibroblasts (FIG. 19E). Compound 1 (10 μM) increased N370S/V394L GC activity to a maximum of 3.6-fold, whereas compound 2 (10 μM) afforded a 2.8-fold increase, more than the increases observed with L444P cells. High concentrations of compounds 5 and 6 (>10 μM) are toxic to both L444P and N370S/V394L fibroblasts. At 100 μM, compounds 1-4 also lower GC activity due to cytotoxicity.

FIG. 19 shows intracellular Ca²⁺ ion concentration influences GC activity in L444P and N370S/V394L GC fibroblasts. (A) Variable Ca²⁺ ion cell culture media concentrations were applied to L444P GC cells for 10 days and to N370S/V394 GC cells for 7 days before using the lysed cell GC activity assay. The GC activity was normalized to that with 2 mM Ca added in the media in both cases. (B) L444P and N370S/V394L GC cells were lysed and equal amount of total cell protein was incubated with Ca²⁺ ions and their GC activities were evaluated using the lysed cell GC activity assay. Cerezyme, a recombinant WT GC enzyme, was also tested for its activity after Ca treatment. (C) Chemical structure of diltiazem analogs (compounds 2-6; distinct substructures relative to compound 1 are shown in red) with their reported Ca²⁺ channel blocker IC₅₀ values. Li et al., J Med Chem 35: 3246-3253, 1992. (D) The influence of Ca²⁺ ion channel blockers of varying potency on L444P GC activity. L444P GC cells were incubated with compounds 1-6 for 7 days before using the intact cell GC activity assay to evaluate lysosomal GC activity. (E) The influence of Ca ion channel blockers of varying potency on N370S/V394L GC activity. N370S/V394L GC cells were incubated with compounds 1-6 for 7 days before evaluating GC activity using the intact cell activity assay. For both (D) and (E), the GC activity of treated cells was normalized against that of untreated cells of the same type (left y axis) and expressed as the percentage of WT GC activity (right y axis).

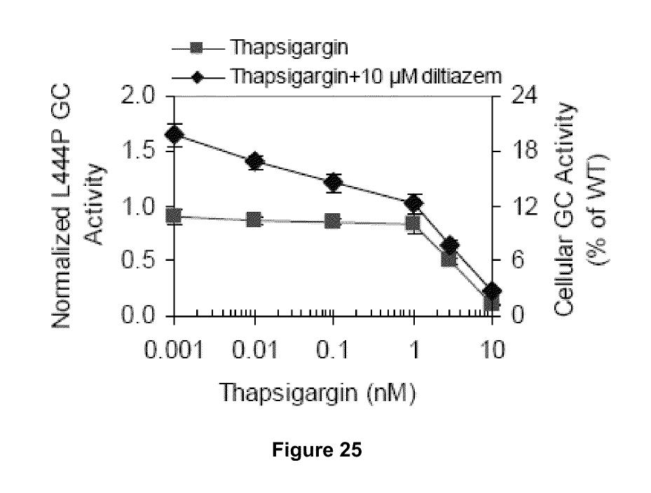

To further investigate the idea that diltiazem enhances GC activity by blocking plasma membrane Ca²⁺ ion channels, thus lowering intracellular Ca²⁺ concentrations, thapsigargin, a potent SERCA pump inhibitor, was applied to L444P GC cells without or with 10 μM diltiazem for 7-days. Thapsigargin inhibits Ca²⁺ entry into the ER from the cytosol, presumably leading to an increase in intracytoplasmic Ca²⁺ ion concentrations. Egan et al., Nat Med 8: 485-492, 2002. Therefore, diltiazem and thapsigargin have opposite effects on regulating cytosolic calcium homeostasis. Thapsigargin alone had no influence on GC activity until a concentration of 1 nM was reached; above this concentration thapsigargin decreased L444P GC activity significantly after 7-day incubation (FIG. 25, pink line). Co-application of varying concentrations of thapsigargin with 10 μM diltiazem revealed a thapsigargin dose-dependent decrease of GC activity (FIG. 25, blue line), consistent with the hypothesis that these compounds have opposite influences on cytoplasmic Ca²⁺ ion levels and that lower rather than higher intracellular Ca²⁺ levels enhance mutant GC homeostasis.

FIG. 25 shows the influence of thapsigargin and diltiazem on GC activity in L444P GC fibroblasts. Thapsigargin was applied without or with 10 μM diltiazem for 7 days. The GC activity of treated cells was normalized against that of untreated L444P GC cells (left y axis) and expressed as the percentage of WT GC activity (right y axis).

### Example 20

### Diltiazem Treatment Upregulates the Expression of Chaperones

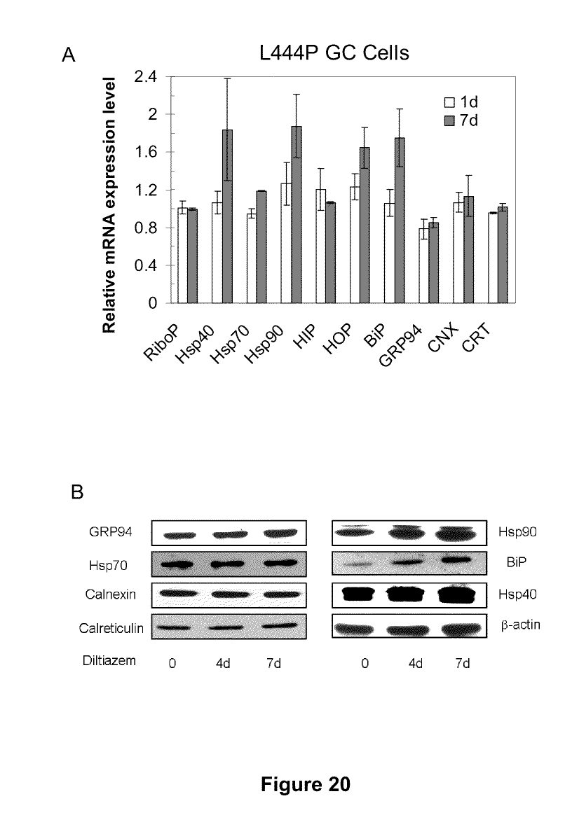

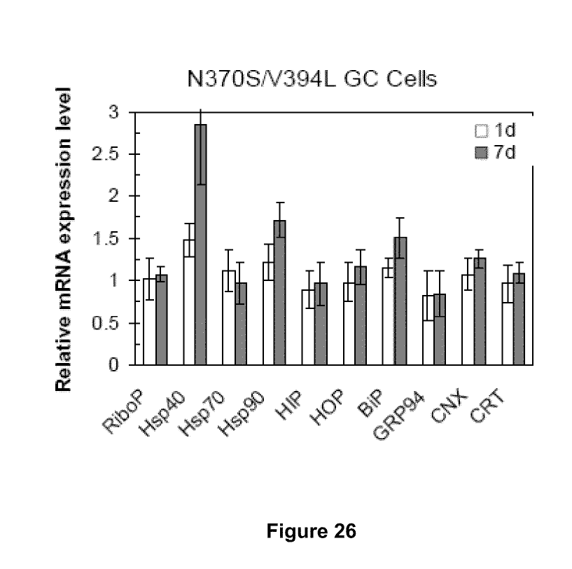

Molecular chaperones are known to be essential for the maintenance of cellular protein homeostasis; hence it is possible that elevated chaperone expression levels could be responsible for the observed diltiazem-mediated enhancement in lysosomal enzyme homeostasis. Ron et al., Nat Rev Mol Cell Biol 8: 519-529, 2007; Young et al., Nat Rev Mol Cell Biol 5: 781-791, 2004; Bukau et al., Cell 125: 443-451, 2006; Williams, J Cell Sci 119: 615-623, 2006. Quantitative RT-PCR analysis was performed on L444P GC fibroblasts incubated without and with 10 μM diltiazem for 1 day and 7 days. The relative mRNA expression levels of representative cytoplasmic and ER lumenal chaperones, including Hsp40, Hsp70, BiP, Hsp90, GRP94, calnexin, calreticulin, HIP and HOP, were probed and normalized to the levels found in untreated cells (FIG. 20A). The large ribosomal protein (RiboP) was monitored as a control. All the primer pairs used are listed in Table 3. The mRNA expression levels of BiP, Hsp40, and Hsp90 were increased up to 1.8-fold, 1.8-fold, and 1.9-fold, respectively, after a 7-day treatment with diltiazem, whereas the mRNA expression levels of Hsp70, GRP94, calnexin, and calreticulin were not changed significantly. A strictly analogous RT-PCR analysis of N370S/V394L GC fibroblasts reveals similarly increased mRNA expression levels of Hsp40, however BiP and Hsp90 exhibit less of an increase after 7 days of diltiazem treatment (FIG. 26). Western blot analysis was also performed on L444P GC fibroblasts incubated without and with diltiazem (10 μM) for 4 days and 7 days (FIG. 20B). The increased protein expression levels of BiP, Hsp40, and Hsp90 were confirmed after a 7-day treatment with diltiazem. That the expression levels of GRP94, Hsp70, calnexin, and calreticulin were not changed significantly was also confirmed at the protein level. These increases in molecular chaperone expression levels, especially the cytoplasmic Hsp40 levels, seem to account for the increased GC folding capacity of the ER, and the requirement for new transcription may also contribute to the relatively slow increases in lysosomal enzyme levels upon calcium channel blocker treatment. Given the highly dynamic nature of the ER, it is envisioned that the cytosolic chaperones play a role in creating an ER optimized for protein folding and trafficking.

FIG. 20 shows chaperone expression level in untreated and diltiazem-treated L444P GC fibroblasts. (A) Quantitative RT-PCR on untreated and diltiazem-treated L444P GC cells. L444P GC cells were incubated with 10 μM diltiazem for 1 day and 7 days, respectively. Relative mRNA expression level for diltiazem-treated L444P GC cells was normalized to that of untreated cells. Hsp40, Hsp70, Hsp90, HIP, HOP, BiP, GRP94, calnexin, and calreticulin were probed using corresponding primer pairs. Large ribosomal protein (RiboP) served as a housekeeping control. (B) L444P GC cells were treated with 10 μM diltiazem for 4 days and 7 days before being lysed for SDS-PAGE analysis, respectively. Hsp40, Hsp70, Hsp90, BiP, GRP94, calnexin, and calreticulin were probed using western blot analysis. β-actin served as a loading control.

FIG. 26 shows quantitative RT-PCR analysis on untreated and diltiazem-treated N370S/V394L GC cells. N370S/V394L GC cells were incubated with 10 μM diltiazem for 1 day and 7 days, respectively. Relative mRNA expression level for diltiazem-treated N370S/V394L GC cells was normalized to that of untreated cells. Hsp40, Hsp70, Hsp90, HIP, HOP, BiP, GRP94, calnexin (CNX), and calreticulin (CRT) were probed using corresponding primer pairs. Large ribosomal protein (RiboP) served as a housekeeping gene control.

### Example 21

### Ca²⁺ Channel Blockers Improve Enzyme Homeostasis in Two Additional Lysosomal Storage Diseases Associated with Glycoprotein and Heparan Sulfate Accumulation

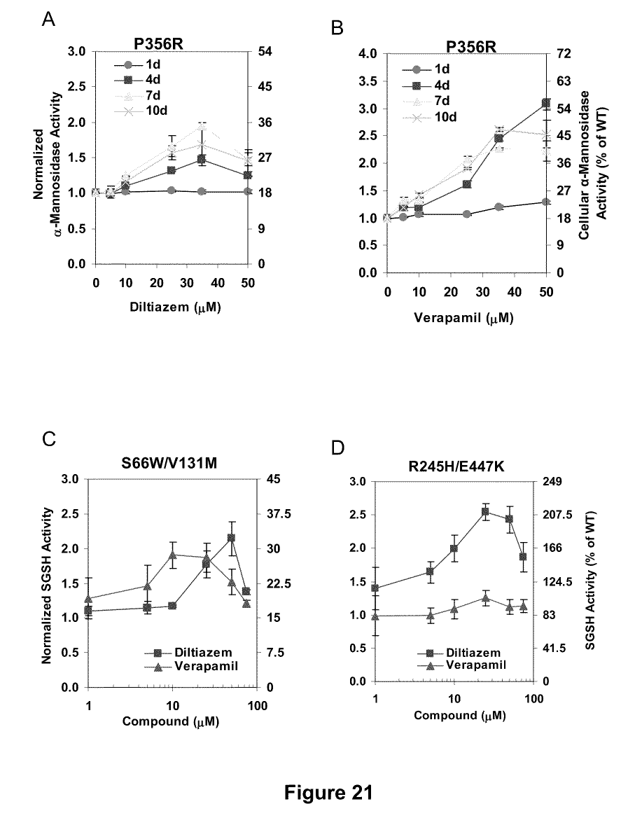

Lysosomal α-mannosidase is a broad specificity exoglycosidase involved in the ordered degradation of glycoproteins. Michalski et al, Biochim Biophys Acta-Mol Basis Dis 1455: 69-84, 1999. The P356R mutation in the α-mannosidase enzyme appears to compromise folding and trafficking, leading to very low lysosomal α-mannosidase activity and severe α-mannosidosis. Gotoda et al., Am J Hum Genet 63: 1015-1024, 1998. The activity of cells harboring P356R α-mannosidase is approximately 18% of that of WT α-mannosidase, under the assay conditions employed. Incubation of these cells with a range of diltiazem or verapamil concentrations for 1, 4, 7 and 10 days enabled lysed cell enzyme activity analysis to be performed. Diltiazem (35 μM) increased the P356R α-mannosidase activity up to 2.0-fold after 7-day incubation period (≈36% the activity of WT α-mannosidase; FIG. 21A). Verapamil (50 μM) increased the P356R α-mannosidase activity up to 3.1-fold (≈56% the activity of WT α-mannosidase) after an incubation period of 4 days, FIG. 21B. Brief exposure of P356R α-mannosidase harboring cells to diltiazem or verapamil (1 day) did not increase α-mannosidase activity significantly (FIGS. 21A and 21B), indicating that it is likely that new protein synthesis is required for diltiazem and verapamil to affect cellular protein homeostasis, consistent with the result obtained from Gaucher cell lines described above (FIGS. 17 and 24).

The lysosomal storage disease mucopolysaccharidosis (MPS) type IIIA is caused by a deficiency of the enzyme sulfamidase (SGSH), resulting in the defective degradation and storage of heparan sulfate, a glycosaminoglycan. Yogalingam et al., Hum Mutat 18: 264-281, 2001. The common S66W and R245H mutations in type IIIA MPS lead to reduced specific activity (15% and 83% of normal specific activity for S66W and R245H, respectively) and lower cellular concentrations, likely a result of compromised folding and trafficking of the sulfamidase variants to the lysosome. Perkins et al., J Biol Chem 274: 37193-37199, 1999. Two compound heterozygous MPS cell lines were utilized to evaluate the effect of diltiazem or verapamil, using an intact cell enzyme activity assay. In the case of the S66W/V131M MPS cells, diltiazem (50 μM) or verapamil (10 μM) treatment increased S66W/V131M sulfamidase activity up to 2.1-fold and 1.9-fold (≈30% of WT sulfamidase activity), respectively, after a 5-day treatment, (FIG. 21C). In the case of R245H/E447K MPS cells, diltiazem (25 μM) increased R245H/E447K SGSH activity up to 2.5-fold (≈207% of WT sulfamidase activity), whereas verapamil did not change the sulfamidase activity significantly after 5-day treatment (FIG. 21D).

FIG. 21 shows the influence of diltiazem and verapamil on mutant α-mannosidase and heparan sulfate sulfamidase (SGSH) activity in patient-derived fibroblasts. The enzyme activity of treated cells was normalized against that of untreated cells of the same type (left y axis) and expressed as the percentage of WT enzyme activity (right y axis). (A) The influence of diltiazem on P356R α-mannosidase activity after culturing for 1 day (black line), 4 days (pink line), 7 days (blue line), and 10 days (yellow line), respectively. (B) The influence of verapamil on P356R α-mannosidase activity after culturing for 1 day (black line), 4 days (pink line), 7 days (blue line), and 10 days (yellow line), respectively. (C) The influence of diltiazem (pink line) and verapamil (green line) on S66W/V131M SGSH activity after culturing for 5 days, respectively. (D) The influence of diltiazem (pink line) and verapamil (green line) on R245H/E447K SGSH activity after culturing for 5 days, respectively. Unlike in FIGS. 17 and 19, % activity relative to WT in FIGS. 21C and 21D is calculated from specific activity of S66W and R245H reported in the literature. Perkins et al., J Biol Chem 274: 37193-37199, 1999.

### Example 22

### L-type Ca²⁺ Channel Blockers Restore Partial Folding, Trafficking and Enzyme Function

The results presented herein relate to the discovery that the L-type Ca²⁺ channel blockers diltiazem and verapamil restore partial folding, trafficking and enzyme function to patient-derived fibroblasts in three distinct lysosomal storage diseases, disorders involving deficiencies in nonhomologous lysosomal enzymes that perform distinct chemical reactions. That these Ca²⁺ channel blockers are both FDA-approved drugs provides the incentive to conduct further necessary efficacy and safety experiments to discern whether they are promising candidates to ultimately treat neuropathic Gaucher disease, and related LSDs. Fortunately, diltiazem crosses the blood-brain barrier, and is bioavailable in the μM concentration range in blood plasma. Naito et al., Arzneimittelforschung 36-1: 25-28, 1986; Buckley et al., Drugs 39: 757-806, 1990.

The Ca²⁺ ion channel blocker potency of diltiazem and its analogs correlates with their efficiency to enhance GC folding in the ER, enabling trafficking and the lysosomal localization of mutant GC in patient-derived fibroblast cell lines. Kraus et al., J Biol Chem 273: 27205-27212, 1998. But how is the blockage of L-type Ca channels on the plasma membrane by diltiazem coupled to enhanced mutant GC homeostasis? Activation of these channels allows extracellular Ca²⁺ to enter the cytosol, which subsequently induces further Ca ion release from intracellular Ca stores, such as the ER, by activating ryanodine receptors, the Ca²⁺ ion channels within the ER membrane. Inhibiting this calcium-induced calcium release (CICR) pathway minimizes depletion of the ER Ca store, a process that appears to upregulate the expression of a subset of cytosolic and ER chaperones, especially Hsp40. Putney et al., Cell Mol Life Sci 57: 1272-1286, 2000.

Others have reported that the reduction in ER Ca²⁺ ion concentrations by SERCA pump inhibitors, such as curcumin and thapsigargin, enhance folding and trafficking of ΔF508 CFTR. Egan et al., Science 304: 600-602, 2004; Egan et al., Nat Med 8: 485-492, 2002. However, thapsigargin does not enhance L444P GC folding, trafficking and lysosomal activity (FIG. 25). Nor does diltiazem treatment increase the trafficking of ΔF508 CFTR to the plasma membrane. Diltiazem blocks calcium entry into the cytosol while thapsigargin inhibits calcium movement from the cytosol into the ER. Therefore, diltiazem and thapsigargin regulate calcium homeostasis oppositely, presumably explaining why diltiazem and thapsigargin partially correct defective protein homeostasis in Gaucher disease and Cystic Fibrosis, respectively.

Diltiazem is an FDA-approved small molecule used to treat angina and hypertension marketed under names including Cardizem, Dilacor, and Tiazec. Unlike pharmacological chaperones that directly bind to GC, thus stabilizing the folded enzyme in the ER for trafficking to the Golgi and on to the lysosome, diltiazem treatment of fibroblasts derived from Gaucher patients appears to alter the biological folding capacity of the ER. Diltiazem is well-tolerated and the incidence of side effects is low. Its pharmacological properties have been extensively studied and reviewed. Buckley et al., Drugs 39: 757-806, 1990; Tartaglione et al., Drug Intell Clin Pharm 16: 371-379, 1982; Chaffman et al., Drugs 29: 387-454, 1985. While diltiazem exhibited its best efficacy at increasing GC activity in patient-derived fibroblasts when utilized at a culture concentration of 10 μM, its lowest effective cell culture media concentration is in the range of 0.1 μM to 1 μM (FIGS. 18 and 19), equivalent to human plasma levels achieved by oral dosing.

Diltiazem and verapamil, potent FDA approved L-type Ca²⁺ channel blocker drugs, increased the ER folding capacity, trafficking and activity of mutant lysosmal enzymes associated with three distinct lysosomal storage diseases. These compounds likely act through a Ca²⁺ ion mediated ER upregulation of a subset of cytoplasmic and ER lumenal chaperones. Increasing ER calcium levels appears to be a relatively selective strategy to partially restore mutant lysosomal enzyme homeostasis in patient-derived cells, as ΔF508 CFTR folding efficiency and the folding efficiency of several other cellular WT enzymes was unaffected by these Ca²⁺ channel blockers.

### Example 22

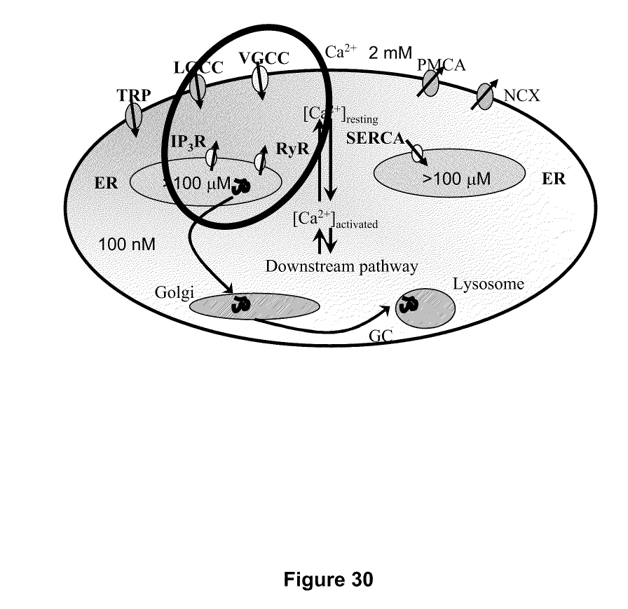

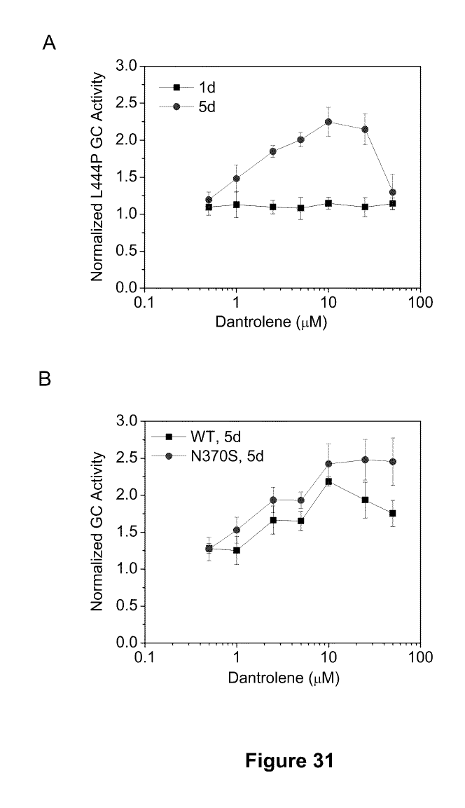

To further study the significance of ER Ca²⁺ homeostasis on the folding, trafficking and function of mutant proteins, ER Ca²⁺ levels were modulated by targeting three systems: IP₃ receptors (IP₃R), ryanodine receptor (RyR) release channels, and the sarco/endoplasmic reticulum Ca²⁺-ATPase (SERCA) pump (FIG. 30).

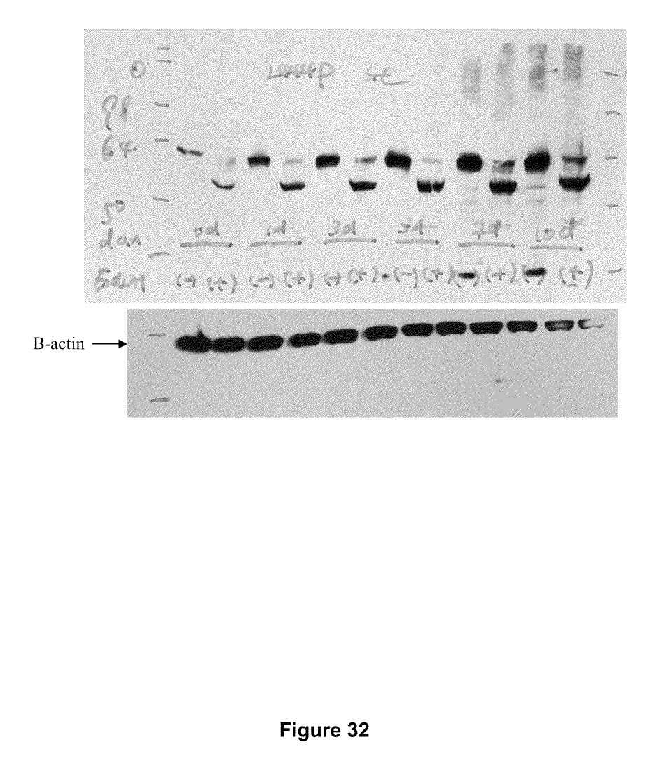

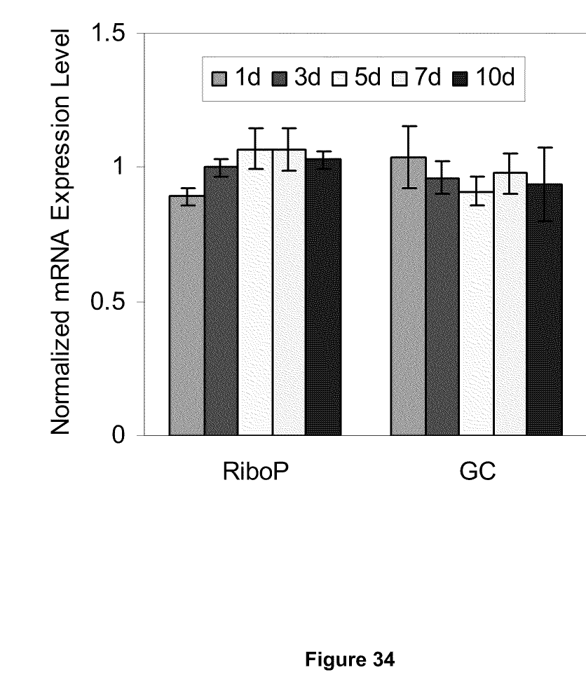

A ryanodine receptor antagonist, dantrolene, was tested for its effect on GC activity in L444P GC (FIG. 31A) and N370S GC (FIG. 31B) fibroblasts. Dantrolene potently blocks ryanodine receptors (RyR) in the ER membrane and thereby inhibits Ca²⁺ release from the ER and increases ER Ca²⁺ levels. Dantrolene significantly increased levels of L444P GC activity (31A and 31B) without significantly increasing GC mRNA expression levels (FIG. 34), indicating that ryanodine receptor antagonists are proteostasis regulators (PR) of GC. The glycosylation of L444P GC fibroblasts exposed to dantrolene (FIG. 32) indicates that dantrolene enhances folding and/or trafficking of L444P GC, providing further support for the role of dantrolene as a PR of mutant GC.

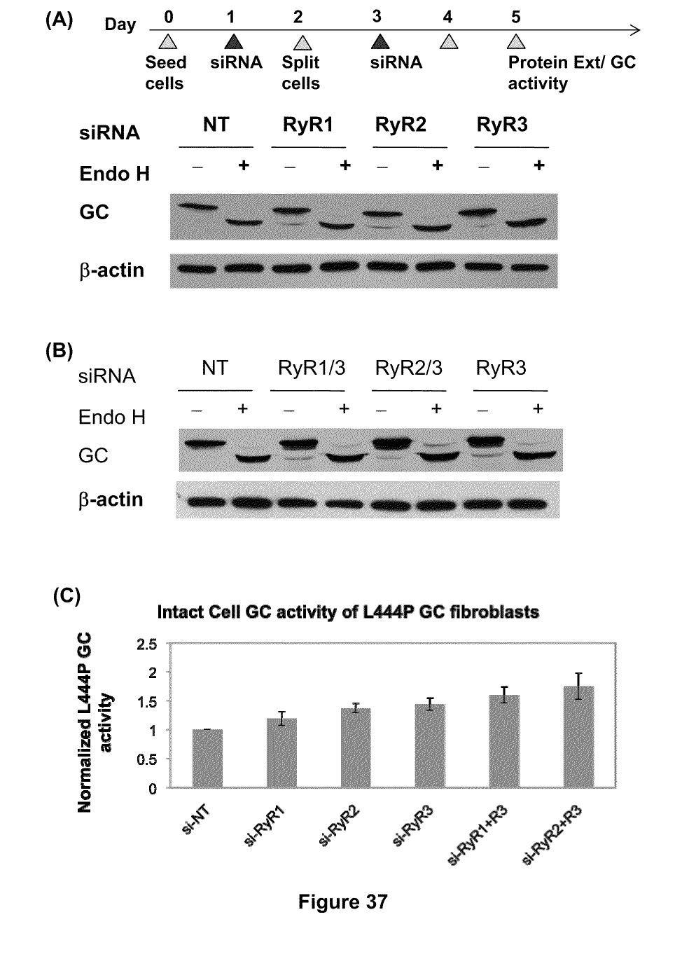

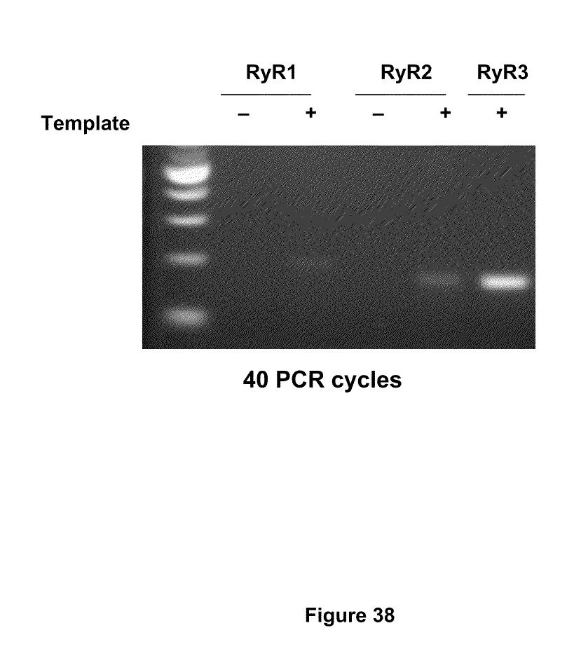

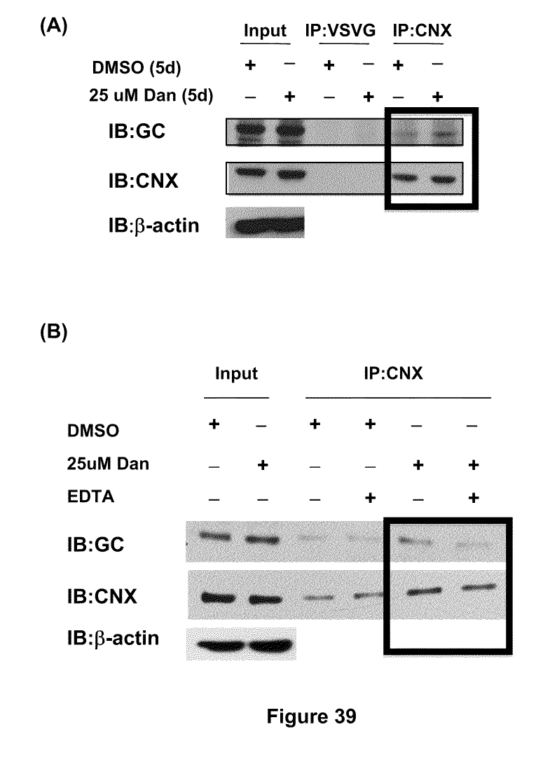

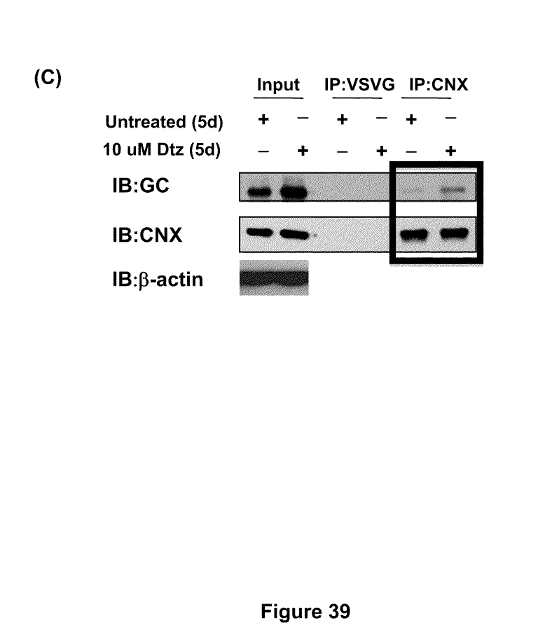

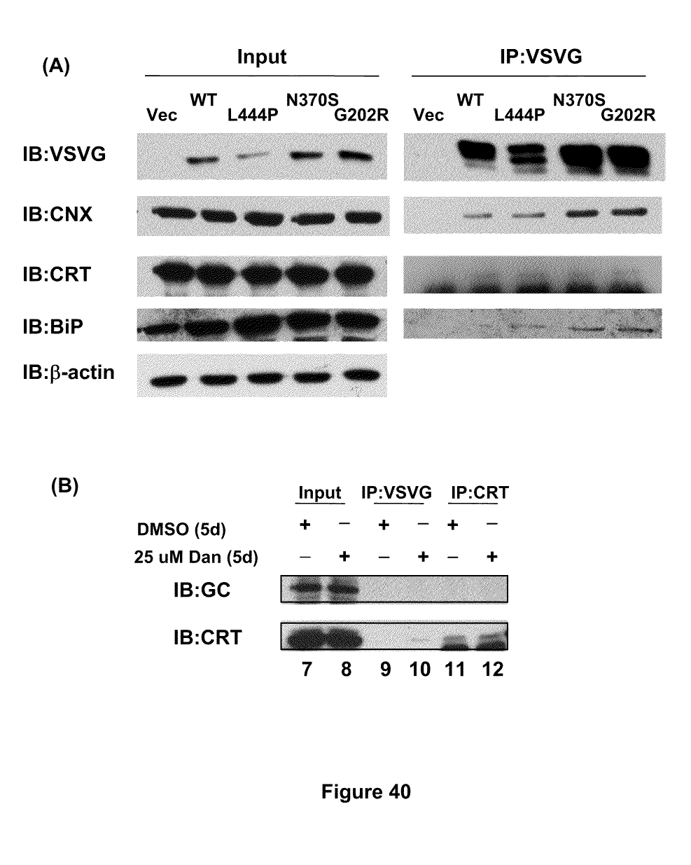

L444P GC fibroblasts were exposed to siRNA against individual ryanodine receptors (RyR1-RyR3) and combinations thereof, and GC activity (FIG. 37C) and Endo H sensitivity (FIG. 37A and FIG. 37B) were measured. Results suggested that PR acting on the RyR isoforms, both individually and in combination, can partially restore L444P GC protein homeostasis, e.g., by promoting GC folding and/or trafficking. The potential PR targets included RyR3, which is the most abundantly expressed isoform in L444P GC fibroblasts (FIG. 38).

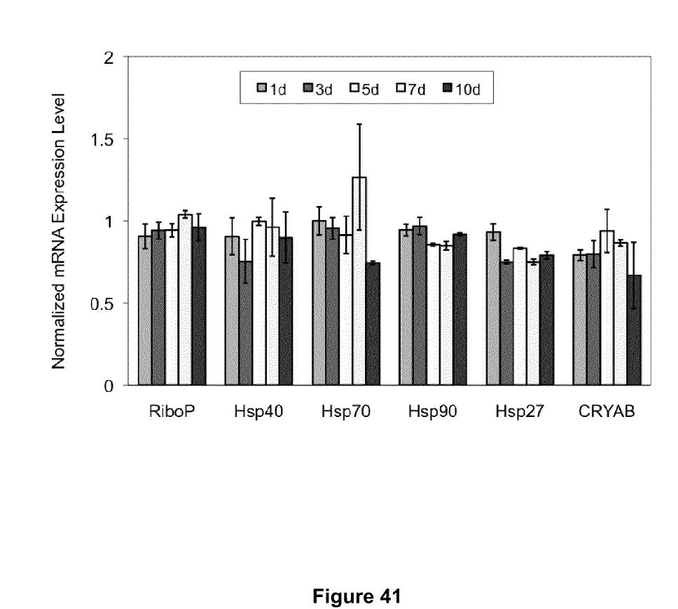

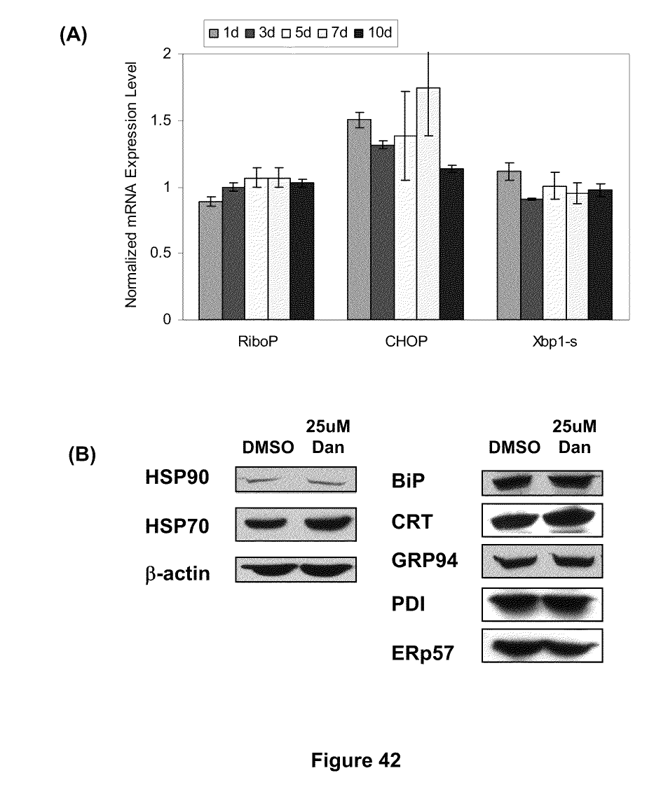

To explore the mechanism dantrolene's PR activity, relative expression levels of the cytoplasmic chaperones Hsp40, Hsp70, Hsp90, Hsp27, and αβ-crystallin (CRYAB) were measured in L444P GC fibroblasts after varying exposures to dantrolene (FIG. 41). Dantrolene does not appear to significantly activate cytoplasmic chaperones.

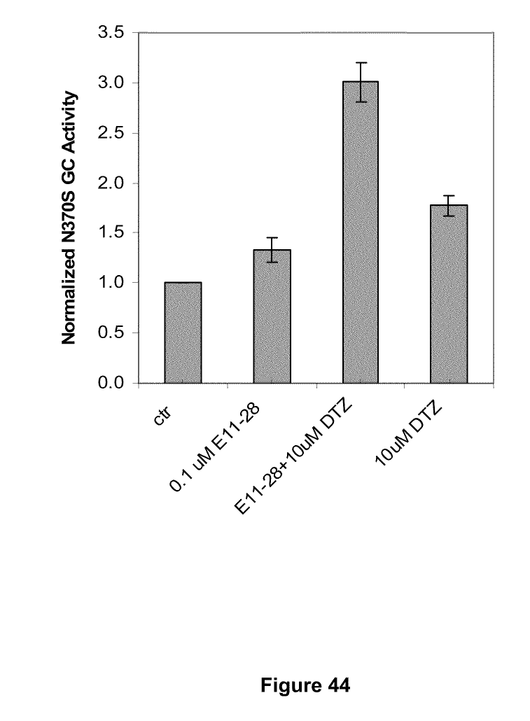

Having demonstrated that dantrolene is a PR of GC, we investigated the effect of dantrolene in combination with a pharmacologic chaperone. The GC activity of N370S GC fibroblasts was measured in the presence of dantrolene and dantrolene in combination with a pharmacological chaperone (PC). Both dantrolene and PC significantly enhanced N370S GC activity, and the combination of dantrolene and PC synergistically enhanced N370S GC activity to an extent greater than the sum of the individual compounds (FIG. 44).

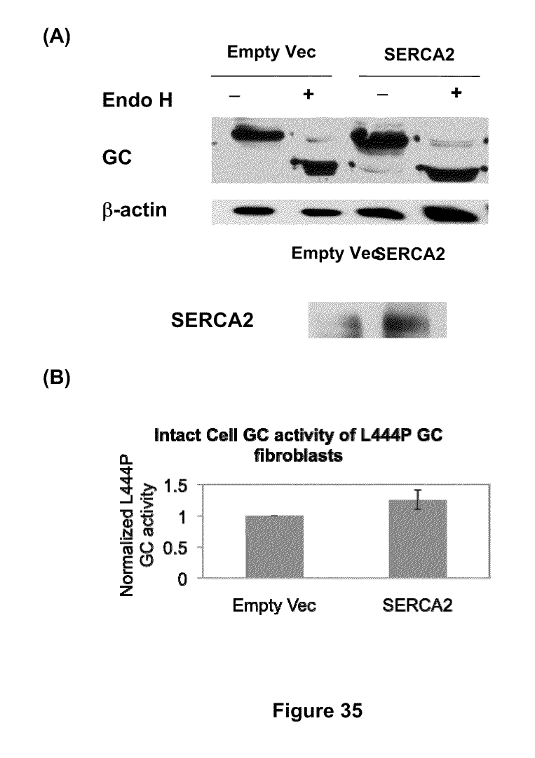

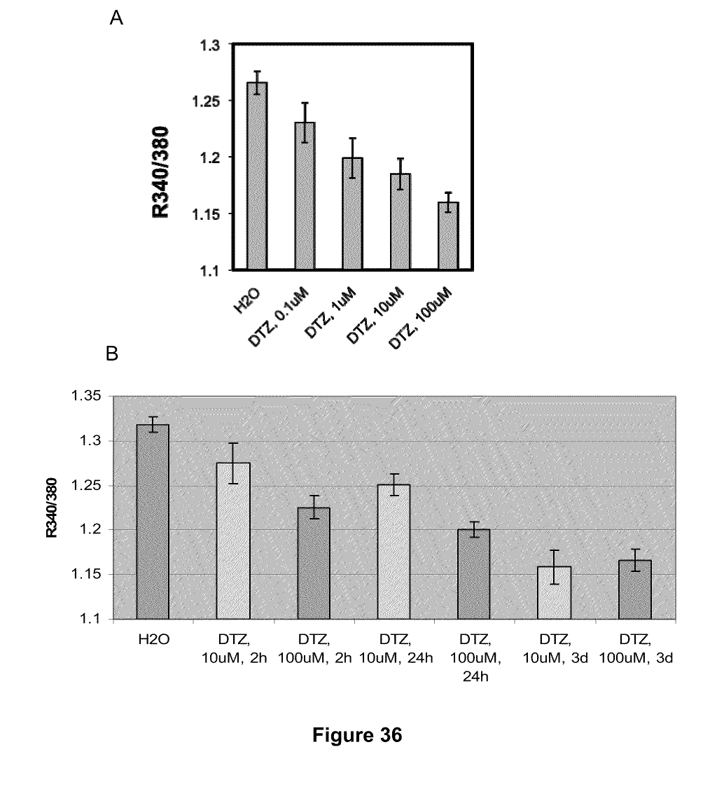

The effect of ER Ca²⁺ levels on the proteostasis of mutant GC was further investigated by overexpressing the SERCA pump in L444P GC fibroblasts and measuring GC glycosylation (FIG. 35A) and GC activity (FIG. 35B). While SERCA overexpression had a modest effect on the activity of L444P GC, folding and trafficking of L444P GC was significantly enhanced. These results further support the finding that raising ER Ca²⁺ levels enhances proteostasis, e.g., by upregulating ER chaperone levels and/or activity.

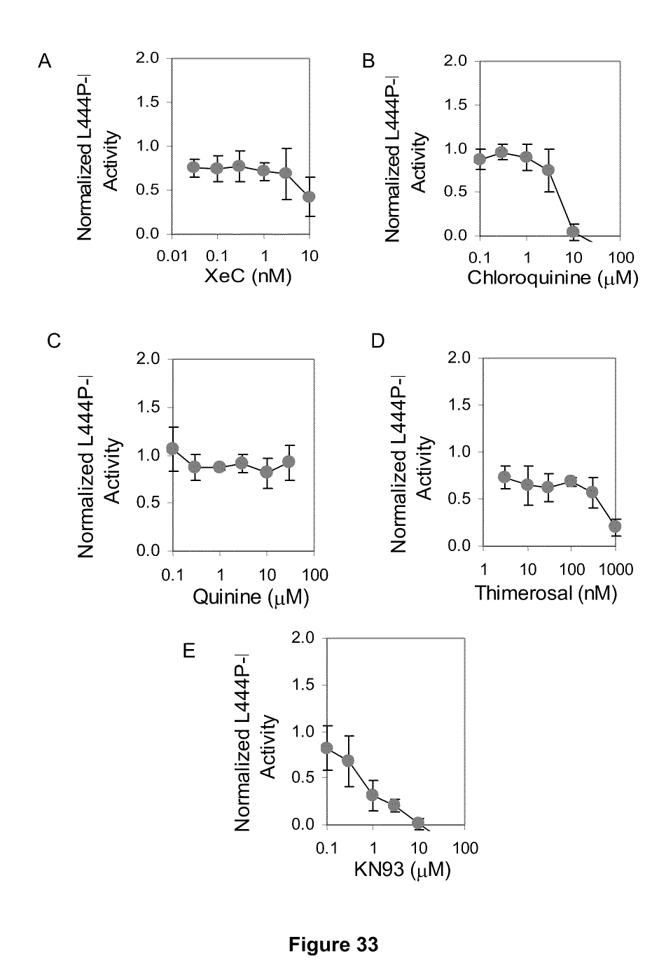

Another potential avenue for modulating ER Ca²⁺ levels is through the inositol triphosphate (IP₃) signaling pathway. IP₃ together with diacylglycerol binds to and activates IP₃ receptors on the ER membrane, causing Ca²⁺ channels in the sarcoplasmic reticulum (SR) to open and release calcium into the cytoplasm and sarcoplasm. The increase in Ca²⁺ concentrations acts as a positive feedback mechanism that in turn stimulates ryanodine receptors in the SR to release additional Ca²⁺. To test whether modulation of the IP₃R pathway regulates proteostasis, the GC activity of L444P GC fibroblasts was measured in the presence of several IP₃R modulators (FIG. 33), including the IP₃R inhibitors XeC (33A), chloroquinine (33B), quinine (33C), thimerosal (33D) and KN93 (33E). None of the compounds enhanced L444P GC activity, and several of the compounds significantly decreased L444P GC activity at micromolar levels.

### Example 24

### Materials and Methods

Reagents. Diltiazem hydrochloride (1) and verapamil were from Tocris Bioscience (Ellisville, Mo.). Compound 2 was from Synfine (Richmond Hill, ON, Canada). Compounds 3 and 4 were synthesized as in supporting information. Ruthenium red, compounds 5 and 6,4-methylumbelliferyl β-D-glucopyranoside, 4-methylumbelliferyl α-D-mannopyranoside, 4-methylumbelliferyl α-D-glucopyranoside, 4-methylumbelliferyl β-D-galactopyranoside, 4-methylumbelliferyl α-D-galactopyranoside, and 4-methylumbelliferyl β-D-glucuronide were from Sigma (St. Louis, Mo.). N-(n-nonyl)deoxynojirimycin (NN-DNJ), Conduritol B epoxide (CBE), and 4-methylumbelliferyl 2-sulfamino-2-deoxy-α-D-glucopyranoside were from Toronto Research Chemicals (Downsview, ON, Canada). All the other tested small molecules were either from Tocris Bioscience or from Sigma. Cell culture media were obtained from Gibco (Grand Island, N.Y.). Human injection quality recombinant WT GC protein (trade name Cerezyme) was obtained from Genzyme (Cambridge, Mass.).

Cell cultures. Primary skin fibroblast cultures were established from Gaucher patients homozygous for either the N370S GC (c. 1226A>G) mutation or the G202R GC (c.721G>A) mutation. An apparently normal fibroblast (GM00498), three distinct homozygous Gaucher fibroblasts containing the L444P GC (c.1448T>C) mutation (GM08760, GM10915, and GM20272), two compound heterozygous Gaucher fibroblasts containing the N370S/V394L GC mutation (GM01607) and N370S/84GG GC mutation (GM00372), a homozygous α-mannosidosis fibroblast containing the P356R α-mannosidase mutation (GM04518), and two compound heterozygous type IIIA mucopolysaccharidosis fibroblasts containing the S66W/V131M SGSH mutation (GM01881) and R245H/E447K SGSH mutation (GM00879) were obtained from the Coriell Cell Repositories (Camden, N.J.). Fibroblasts were maintained in minimum essential medium with Earle's salts supplemented with 10% heat-inactivated fetal bovine serum and 1% glutamine Pen-Strep at 37° C. in 5% CO₂.

Enzyme activity assays. The intact cell GC activity assay has been previously described. Sawkar et al., Proc Natl Acad Sci USA 99: 15428-15433, 2002. Briefly, cells were plated into 48-well assay plates (500 μl per well). After cell attachment, the media was replaced by media containing small molecules. Media was changed every 3 days. After incubation at 37° C. for the indicated amount of time, the intact cell GC activity assay was performed. The monolayers were washed by DPBS. The reaction was started by the addition of 150 μl of 3 mM 4-methylumbelliferyl β-D-glucopyranoside in 0.2 M acetate buffer (pH 4.0) to each well, followed by incubation at 37° C. for 1 hour to 7 hours. CBE was used as a control to evaluate the extent of nonspecific GC activity. The reaction was stopped by lysing the cells with 750 μl of 0.2 M glycine buffer (pH 10.8). Liberated 4-methylumbelliferone was measured (excitation 365 nm, emission 445 nm) with a SpectraMax Gemini plate reader (Molecular Device, Sunnyvale, Calif.). The lysed cell GC activity assay has been previously described. Sawkar et al., Proc Natl Acad Sci USA 99: 15428-15433, 2002. Briefly, intact cells were harvested and the pellet was lysed in the complete lysis-M buffer containing complete protease inhibitor cocktails (Roche #10799050001). Total cell protein was measured using the Micro BCA assay reagent (Pierce, Rockford, Ill., #23235). 30 μg of total cell protein was assayed for the GC activity in 100 μl of 0.1 M acetate buffer (pH 5.0) containing 3 mM 4-methylumbelliferyl β-D-glucopyranoside in the presence of 0.15% Triton X-100 (v/v, Fisher) and 0.15% taurodeoxycholate (w/v, Calbiochem). CBE was used as a control to evaluate the extent of nonspecific GC activity. After incubation at 37° C. for 1 hour to 7 hours, the reaction was terminated with 200 μl of 0.2 M glycine buffer (pH 10.8), and the fluorescence was recorded (excitation 365 nm, emission 445 nm). The GC activity assay for recombinant WT GC enzymes has been previously described [Sawkar et al., ACS Chem Biol 1: 235-251, 2006]. 25 ng of recombinant WT GC protein was assayed for the GC activity in 50 μl of 0.1 M acetate buffer (pH 5.0) containing 3 mM 4-methylumbelliferyl β-D-glucopyranoside in the presence of 0.15% Triton X-100 (v/v, Fisher) and 0.15% taurodeoxycholate (w/v, Calbiochem). After the addition of tested compounds, the reaction was incubated at 37° C. for 20 min, terminated with 75 μl of 0.2 M glycine buffer (pH 10.8), and the fluorescence was recorded (excitation 365 nm, emission 445 nm).

The activity of lysosomal α-mannosidase was determined as previously described with minor modification by using 2 mM 4-methylumbelliferyl α-D-mannopyranoside as the substrate. Gotoda et al., Am J Hum Genet. 63: 1015-1024, 1998. The activity of lysosomal SGSH was determined by using 0.5 mM 4-methylumbelliferyl 2-sulfamino-2-deoxy-α-D-glucopyranoside as previously described with minor modification. Karpova et al., J Inherit Metab Dis 19: 278-285, 1996. The activities of lysosomal enzymes α-glucosidase, β-galactosidase, α-galactosidase, and β-glucuronidase were assayed as previously described by using corresponding substrates 4-methylumbelliferyl α-D-glucopyranoside, 4-methylumbelliferyl β-D-galactopyranoside, 4-methylumbelliferyl α-D-galactopyranoside, and 4-methylumbelliferyl β-D-glucuronide, respectively. Sawkar et al. Chem Biol 12: 1235-1244, 2005.

Small molecules were evaluated at least in triplicates at each concentration and each molecule was assayed at least three times. The data reported were normalized to the enzyme activity of untreated cells of the same type and expressed as percentage of WT enzyme activity.

Quantitative RT-PCR. The cells were incubated with 10 μM diltiazem at 37° C. for the indicated amount of time. Total RNA was extracted from the cells using RNeasy Mini Kit (Qiagen #74104). cDNA was synthesized from 500 ng of total RNA using QuantiTect Reverse Transcription Kit (Qiagen #205311). Quantitative PCR reactions were performed using QuantiTect SYBR Green PCR Kit (Qiagen #204143) and corresponding primers in the ABI PRISM 7900 system (Applied Biosystems). The forward and reverse primers for GC, Hsp40, Hsp70, Hsp90, HIP, HOP, BiP, GRP94, calnexin (CNX), and calreticulin (CRT), and an endogenous housekeeping gene large ribosomal protein (RiboP) are listed in Table 3. Samples were heated for 15 min at 95° C. and amplified in 45 cycles of 15 s at 94° C., 30 s at 59° C., and 30 s at 72° C. Analysis was done using SDS2.1 software (Applied Biosystems). Threshold cycle (C_{T}) was extracted from the PCR amplification plot. The ΔC_{T} value was used to describe the difference between the C_{T} of a target gene and the C_{T} of the housekeeping gene: ΔC_{T}=C_{T}(target gene)−C_{T}(housekeeping gene). The relative mRNA expression level of a target gene of diltiazem-treated cells was normalized to that of untreated cells: Relative mRNA expression level=2exp[−(ΔC_{T}(treated cells)−ΔC_{T}(untreated cells))].

TABLE 3

| | GenBank | | |
|---|---|---|---|
| | Accession | Forward | Reverse |
| Gene | code | Primer | Primer |
| GC | M16328 | 5′-CTC CAT CCG CAC CTA CAC C-3′ | 5′-ATC AGG GGT ATC TTG AGC TTG |
| | | | G-3′ |
| RiboP | NM_001004 | 5′-CGT CGC CTC CTA CCT GCT-3′ | 5′-CCA TTC AGC TCA CTG ATA ACC |
| | | | TTG-3′ |
| Hsp40 | NM_006145 | 5′-CGC CGA GGA GAA GTT C-3′ | 5′-CAT CAA TGT CCA TGC CTT-3′ |
| Hsp70 | NM_005345 | 5′-GGA GGC GGA GAA GTA CA-3′ | 5′-GCT GAT GAT GGG GTT ACA-3′ |
| Hsp90 | NM_005348 | 5′-GAT AAA CCC TGA CCA TTC C-3′ | 5′-AAG ACA GGA GCG CAG TTT CAT |
| | | | AAA-3′ |
| HIP | NM_003932 | 5′-CCG CAA AGT GAA CGA G-3′ | 5′-TGA TGG TTC GTC TGC C-3′ |
| HOP | NM_006819 | 5′-ATG ACC ACT CTC AGC GTC-3′ | 5′-CTC CTT GGC TTT GTC GTA-3′ |
| BiP | NM_005347 | 5′-GCC TGT ATT TCT AGA CCT | 5′-TTC ATC TTG CCA GCC AGT TG- |
| | | GCC-3′ | 3′ |
| GRP94 | NM_003299 | 5′-GGC CAG TTT GGT GTC GGT TT- | 5′-CGT TCC CCG TCC TAG AGT |
| | | 3′ | GTT-3′ |
| CNX | NM_001746 | 5-GCG TTG TGG GGC AGA TGA T-3′ | 5′-CCG GTT GAG GTG CAT CAG T-3′ |
| CRT | NM_004343 | 5′-AAG TTC TAC GGT GAC GAG | 5′-GTC GAT GTT CTG CTC ATG TTT |
| | | GAG-3′ | C-3′ |

Western blot. The cells were lysed using the complete lysis-M buffer containing complete protease inhibitor cocktails (Roche #10799050001). Total cell protein was measured using the Micro BCA assay reagent. Endo H (New England Biolabs #P0703) was used to digest the cell lysates according to the company instructions. The cell lysates containing equal amount of total protein were separated by 10% SDS-PAGE. Western blot analysis was performed with antibodies, mouse monoclonal anti-GC 8E4. Ginns et al., Clin Chim Acta 131: 283-287, 1983. Mouse monoclonal anti-GC 2E2 was from Novus Biologicals (#H00002629-MO 1, Littleton, Colo.). Antibodies directed against calnexin (#SPA-860), calreticulin (#SPA-601), Hsp40 (#SPA-400), Hsp70 (#SPA-812), and Hsp90 (#SPA-830) were from Stressgen (Victoria, BC, Canada). Antibodies directed against BiP (#SC-13968) and GRP94 (#SC-11402) were from Santa Cruz Biotechnology (Santa Cruz, Calif.). Mouse monoclonal anti-β actin AC-15 was from Sigma (#A1978). Secondary antibodies (#31430 for goat anti-mouse and #31460 for goat anti-rabbit) were from Pierce. Bands were visualized using the SuperSignal West Pico Chemiluminescent Substrate (Pierce #34078) or SuperSignal West Femto Maximum Sensitivity Substrate (Pierce #34095).

Immunofluorescence. Immunofluorescence has been previously described. Sawkar et al., ACS Chem Biol 1: 235-251, 2006. Cells grown on cover glass slips were washed by PBS and fixed with 3.7% paraformaldehyde in PBS for 15 min. The slips were washed with PBS, quenched with 15 mM glycine in PBS for 10 min, and permeabilized with 0.2% saponin in PBS for 15 min. The antibodies were prepared in PBS in the presence of 0.2% saponin and 5% goat serum. Cells were incubated for 1 hour with primary antibodies (1:100 for mouse monoclonal anti-GC 16B3, or 1:100 for 8E4, and 1:10,000 for rabbit anti-LAMP2, washed with 5% goat serum in PBS, and then incubated for 1 hour with secondary antibodies (Alexa Fluor 488 goat anti-mouse IgG (#A11029) and Alexa Fluor 546 goat anti-rabbit IgG (#A11035)) from Molecular Probes (Eugene, Oreg.). Beutler et al., Proc Natl Acad Sci USA 81: 6506-6510, 1984; Carlsson et al., J Biol Chem 263: 18911-18919, 1988. The cover slips were mounted and sealed. Images were collected using a Bio-Rad (Zeiss) Radiance 2100 Rainbow laser scanning confocal microscope attached to a Nikon TE2000-U microscope. For quantitative colocalization analysis, Z-stacks of each frame were flattened and Pearson's correlation coefficient was calculated using NIH Image J software. Random frames from each slide were averaged and colocalization differences were analyzed using a two-tailed Student's t-test.

Syntheses and structural characterization of compounds 3 and 4. Reagents were purchased from Aldrich. NMR spectra were recorded on a Varian FT NMR spectrometer at a proton frequency of 400 MHz. High-resolution mass spectra (HRMS) were obtained at The Scripps Research Institute Center for Mass Spectrometry. High performance liquid chromatography (HPLC) separations were performed on a Waters dual 600 pump liquid chromatography system equipped with a Waters 2487 PDA (photodiode array) UV detector using a Phenomenex Jupiter 4u Proteo 90A reverse phase C18 column (250×21.20 mm) for preparative HPLC.

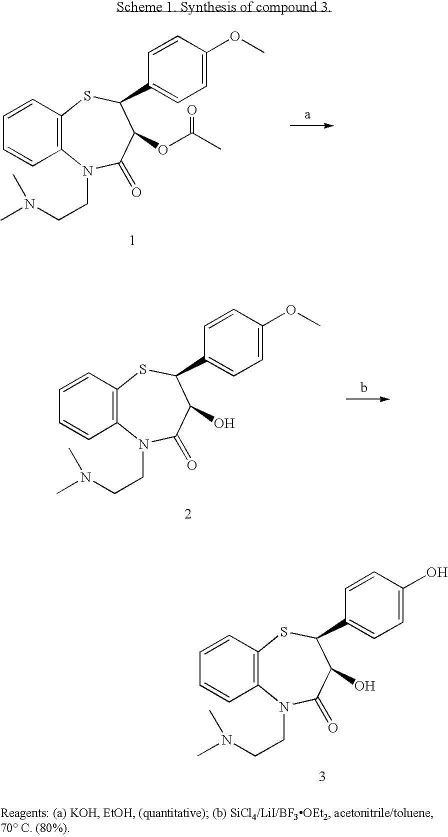

Although the syntheses of compounds 3 and 4 were reported previously, they either required multiple steps or gave low overall yields. Miyazaki et al., Chem Pharm Bull 26: 2889-2893, 1978, Li et al, J Med Chem 35: 3246-3253, 1992. Here we utilized inexpensive commercially available compounds as the starting materials to obtain compounds 3 and 4 in two steps with good yields, respectively. The synthetic route of compound 3 is shown in Scheme 1. Compound 2 was prepared from commercially available diltiazem hydrochloride in quantitative yield. U.S. Pat. No. 4,547,495. Compound 3 was achieved by O-demethylation of 2 in 81% yield with SiCl₄/LiI in the presence of a catalytic amount of BF₃. Zewge et al., Tetrahedron Lett 45: 3729-3732, 2004. The synthetic route of compound 4 is shown in Scheme 2. Compound 7 was prepared from commercially available compound 6 in 71% yield as previously reported. Miyazaki et al., Chem Pharm Bull 26: 2889-2893, 1978. Compound 4 was obtained by both O-demethylation and the removal of the Cbz protecting group from compound 7 in one pot using BBr₃ in 61% yield. McOmie et al., Tetrahedron 24: 2289-2292, 1968;

### Felix, J Org Chem 39: 1427-1429, 1974; Li et al., J Med Chem 35: 3246-3253, 1992.

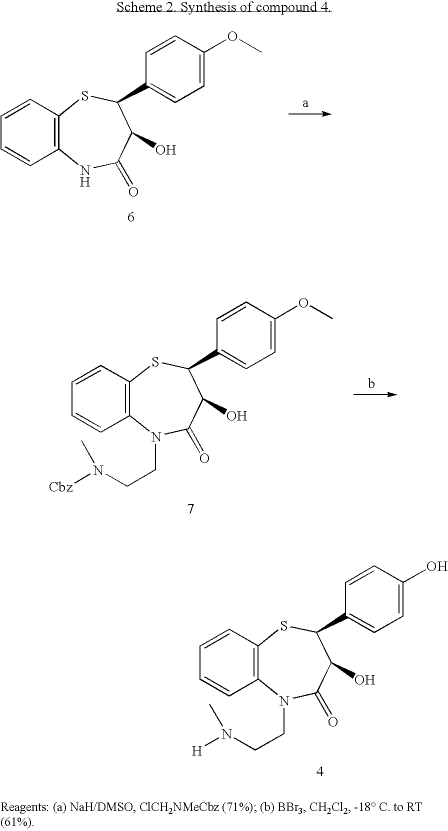

(2S,3S)-5-[2-(Dimethylamino)ethyl]-2,3-dihydro-3-hydroxy-2-(4-hydroxyphenyl)-1,5-benzothiazepin-4(5H)-one (3). 432 mg of 2 (1.16 mmol) was dissolved in 15 ml of anhydrous toluene. 1553 mg of LiI (11.6 mmol) and 5 ml of acetonitrile were added followed by 11.6 ml of 1M SiCl₄ in CH₂Cl₂ (11.6 mmol) and 294 μl of BF₃.OEt₂ (2.32 mmol). The mixture was stirred for 16 h at 70° C. The reaction was quenched by the addition of 25 ml of methanol and excessive solid Na₂CO₃, filtered, and concentrated. The mixture was re-dissolved in 25 ml of CHCl₃ and 25 ml of H₂O. The pH value was adjusted to 9.0 with saturated Na₂CO₃ solution. The mixture was extracted with CHCl₃ (15 ml×3). The combined organic layer was dried over Na₂SO₄, and rotary-evaporated. Flash chromatography (1:9 methanol/CH₂Cl₂) gave 337 mg (81%) of 3 as a pale-yellow powder. ¹H NMR (400 MHz, d₆-DMSO) δ=2.16 (s, 6H), 2.28-2.34 (m, 1H), 2.56-2.61 (m, 1H), 3.67-3.74 (m, 1H), 4.27-4.35 (m, 1H), 4.17 (t, J=7.1 Hz, 1H), 4.41 (d, J=7.4 Hz, 1H), 4.82 (d, J=7.3 Hz, 1H), 6.69-7.67 (m, 8H), 9.43 (s, 1H); ¹³C NMR (100 MHz, d₆-DMSO) δ=45.09, 46.55, 56.05, 56.40, 68.50, 114.50, 124.57, 125.38, 127.11, 128.15, 130.58, 131.17, 134.56, 145.13, 157.17, 170.62; HRMS for C₁₉H₂₂N₂O₃S [M+H]⁺ calc, 359.1424; found, 359.1427.

(2S,3S)-2,3-Dihydro-3-hydroxy-2-(4-hydroxyphenyl)-5-[2-(methylamino)ethyl]-1,5-benzothiazepin-4(5H)-one (4). 138 mg of 7 (0.28 mmol) in 5 ml of anhydrous CH₂Cl₂ was cooled to −18° C. 2 ml of 1M BBr₃ in CH₂Cl₂ (2 mmol) was added dropwise. The reaction was stirred at −18° C. for 1 h and then at room temperature for another 10 h. 10 ml of H₂O was added to the mixture dropwise. The pH value was adjusted to 9.0 with NaOH solution. The mixture was extracted with ethyl acetate (20 ml×3). The combined organic layer was dried over Na₂SO₄, and rotary-evaporated. The crude product was purified by preparative HPLC using a reverse phase C18 column to give 78 mg (61%) of the CF₃COOH salt of 4 as a white powder. ¹H NMR (400 MHz, D₂O) δ=2.76 (s, 3H), 3.28-3.35 (m, 1H), 3.43-3.49 (m, 1H), 4.06-4.13 (m, 1H), 4.43-4.50 (m, 1H), 4.52 (d, J=7.6 Hz, 1H), 4.96 (d, J=7.6 Hz, 1H), 6.90-7.76 (m, 8H); ¹³C NMR (100 MHz, D₂O) δ=33.22, 45.61, 46.85, 55.87, 69.45, 115.46, 124.74, 126.03, 127.86, 128.95, 131.52, 131.60, 135.35, 143.68, 156.18, 173.55; HRMS for C₁₈H₂₀N₂O₃S [M+H]⁺ calc, 345.1267; found, 345.1279.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed:

1. A method for treating a condition characterized by dysfunction in protein homeostasis in a patient in need thereof comprising administering to the patient a proteostasis regulator in an amount effective to improve or restore protein homeostasis, and to reduce or eliminate the condition in the patient or to prevent its occurrence or recurrence.

2. The method of claim 1 wherein said dysfunction in protein homeostasis is a result of protein misfolding.

3. The method of claim 1 wherein said dysfunction in protein homeostasis is a result of protein aggregation.

4. The method of claim 1 wherein said dysfunction in protein homeostasis is a result of defective protein trafficking.

5. The method of claim 1 wherein said dysfunction in protein homeostasis is a result of protein degradation.

6. The method of claim 1 wherein the condition is a loss of function disorder.

7. The method of claim 6 wherein the condition is a lysosomal storage disease.

8. The method of claim 1 wherein the condition is a gain of function disorder.

9. The method of claim 1 wherein the proteostasis regulator upregulates signaling via a heat shock response (HSR) pathway, an unfolded protein response (UPR) pathway, a Ca²⁺ signaling pathway, or a combination thereof.

10. The method of claim 6 wherein the proteostasis regulator upregulates transcription or translation of one or more protein chaperones, one or more folding enzymes, or a combination thereof.

11. The method of claim 6 wherein the proteostasis regulator inhibits degradation of one or more protein chaperones, one or more folding enzymes, or a combination thereof.

12. The method of claim 7 wherein the proteostasis regulator upregulates an aggregation pathway or a disaggregation pathway.

13. The method of claim 6 wherein the condition is Gaucher's disease, α-mannosidosis, type IIIA mucopolysaccharidosis, Fabry disease, Tay-Sach's disease or Pompe disease.

14. The method of claim 6 wherein the loss of function disorder is a lysosomal storage disease resulting from a mutated lysosomal enzyme.

15. The method of claim 15 further comprising administering a polynucleotide or polypeptide encoding a lysosomal enzyme having normal activity to replace the mutated lysosomal enzyme.

16. The method of claim 8 wherein the condition is inclusion body myositis, age-related macular degeneration, amyotrophic lateral sclerosis, Alzheimer's disease, Huntington's disease or Parkinson's disease.

17. The method of claim 1 wherein the proteostasis regulator is a small chemical molecule, a protein, an antisense nucleic acid, short hairpin RNA, short interfering RNA or ribozyme.

18. The method of claim 1 wherein the proteostasis regulator is administered in an amount that does not increase susceptibility of said patient to viral infection.

19. The method of claim 1 wherein the proteostasis regulator is administered in an amount that does not increase susceptibility of said patient to cancer.

20. The method of claim 1 further comprising administering a pharmacologic chaperone or kinetic stabilizer.

21. The method of claim 1 further comprising administering a second mechanistically distinct proteostasis regulator.

22. The method of claim 21 wherein the first and the second proteostasis regulator are one or more of aggregation regulator, disaggregation regulator, protein degradation regulator or protein folding regulator.

23. A method for treating a condition characterized by dysfunction in protein homeostasis in a patient in need thereof comprising administering to said patient a proteostasis regulator in combination with a pharmacologic chaperone or kinetic stabilizer in an amount effective to improve or restore protein homeostasis and to reduce or eliminate the condition in the patient or to prevent its occurrence or recurrence.

24. The method of claim 23 wherein the condition is a loss of function disorder.

25. The method of claim 23 wherein the proteostasis regulator promotes correct folding of a mutated enzyme.

26. The method of claim 25 wherein the mutated enzyme is a lysosomal enzyme.

27. The method of claim 26 further comprising administering a polynucleotide or polypeptide encoding a lysosomal enzyme having normal activity to replace the mutated lysosomal enzyme.

28. The method of claim 23 wherein the proteostasis regulator inhibits endoplasmic reticulum associated degradation.

29. The method of claim 23 wherein the condition is Gaucher's disease.

30. The method of claim 24 wherein the pharmacologic chaperone is N-(n-nonyl)deoxynojirimycin.

31. The method of claim 23 wherein the condition is Tay-Sach's disease.

32. The method of claim 31 wherein the pharmacologic chaperone is 2-acetamido-2-deoxynojirimycin.

33. The method of claim 23 wherein the condition is a gain of function disorder.

34. The method of claim 33 wherein the condition is inclusion body myositis, age-related macular degeneration, amyotrophic lateral sclerosis, Alzheimer's disease, Huntington's disease or Parkinson's disease.

35. A method for treating a loss of function disease in a patient in need thereof comprising administering to said patient a proteostasis regulator in an amount effective to improve or restore activity of a mutated protein and to reduce or eliminate the loss of function disease in the patient or to prevent its occurrence or recurrence.

36. The method of claim 35 wherein said proteostasis regulator promotes correct folding of the mutated protein, and wherein said proteostasis regulator does not bind to the mutated protein.

37. The method of claim 35 wherein said proteostasis regulator reduces or eliminates endoplasmic reticulum associated degradation of a protein chaperone.

38. The method of claim 35 wherein said proteostasis regulator is a proteasome inhibitor.

39. The method of claim 35 wherein the loss of function disease is a lysosomal storage disease and the mutated protein is a lysosomal enzyme.

40. The method of claim 35 wherein the loss of function disease is cystic fibrosis and the mutated protein is cystic fibrosis transmembrane conductance regulator (CFTR).

41. The method of claim 35 further comprising administering a polynucleotide or polypeptide encoding a protein having normal activity to replace the mutated protein.

42. The method of claim 39 wherein the lysosomal storage disease is a neuropathic lysosomal storage disease.

43. The method of claim 39 wherein the lysosomal storage disease is Gaucher's disease, α-mannosidosis, type IIIA mucopolysaccharidosis, Fabry disease, Tay-Sach's disease or Pompe disease.

44. The method of claim 39 wherein the proteostasis regulator increases calcium concentration in the endoplasmic reticulum.

45. The method of claim 44 wherein the proteostasis regulator is a Ca²⁺ channel blocker.

46. The method of claim 44 wherein the proteostasis regulator increases the expression of a calcium binding chaperone protein.

47. The method of claim 46 wherein the calcium binding chaperone protein is selected from the group consisting of calnexin and calreticulin.

48. The method of claim 44 wherein the proteostasis regulator inhibits a ryanodine receptor.

49. The method of claim 48 wherein the proteostasis regulator is a ryanodine receptor antagonist.

50. The method of claim 48 wherein the proteostasis regulator inhibits expression of the ryanodine receptor.

51. The method of claim 48 wherein the proteostasis regulator inhibits at least two ryanodine receptor subtypes.

52. The method of claim 39 wherein the proteostasis regulator is dilitiazem or verapamil.

53. The method of claim 43 wherein the lysosomal storage disease is Gaucher's disease.

54. The method of claim 53 wherein the lysosomal storage disease is neuropathic Gaucher's disease.

55. The method of claim 53 wherein the enzyme is glucocerebrosidase.

56. The method of claim 55 wherein the enzyme is L444P glucocerebrosidase.

57. The method of claim 55 wherein the enzyme is N370S glucocerebrosidase.

58. The method of claim 43 wherein the lysosomal storage disease is α-mannosidosis.

59. The method of claim 58 wherein the enzyme is α-mannosidase.

60. The method of claim 59 wherein the enzyme is P356R α-mannosidase.

61. The method of claim 43 wherein the lysosomal storage disease is type IIIA mucopolysaccharidosis.

62. The method of claim 61 wherein the enzyme is sulfamidase.

63. The method of claim 62 wherein the enzyme is S66W sulfamidase or R245H sulfamidase.

64. The method of claim 39 wherein the proteostasis regulator is a Ca²⁺ channel antagonist.

65. The method of claim 64 wherein the proteostasis regulator is an L-type voltage gated calcium channel blocker.

66. The method of claim 65 wherein the proteostasis regulator is diltiazem or verapamil.

67. The method of claim 65 wherein the proteostasis regulator is an analog of dilitiazem.

68. The method of claim 43 wherein the disease is Tay-Sach's disease.

69. The method of claim 68 wherein the enzyme is β-hexosamine A.

70. The method of claim 69 wherein the enzyme is G269S β-hexosamine A.

71. The method of claim 70 wherein the proteostasis regulator is celastrol.

72. The method of claim 70 wherein the proteostasis regulator is MG-132.

73. A method for treating a condition characterized by a dysfunction in protein homeostasis in a patient in need thereof comprising administering to said patient at least two mechanistically distinct proteostasis regulators wherein said proteostasis regulators are administered in an amount effective to improve or restore protein homeostasis and to reduce or eliminate the condition in the patient or to prevent its occurrence or recurrence.

74. The method of claim 73 wherein one of said proteostasis regulators enhances correct folding of a mutated protein.

75. The method of claim 73 wherein one of said proteostasis regulators inhibits endoplasmic reticulum associated degradation of a mutated protein.

76. The method of claim 73 wherein the mutated protein is a mutated enzyme.

77. A method for diagnosing a condition characterized by a dysfunction in protein homeostasis in a patient comprising,
contacting cells or tissue from the patient with a proteostasis regulator in a cell-based assay system,
measuring an effect of the proteostasis regulator on protein folding, protein aggregation, protein trafficking or protein degradation in the cell, and
identifying a deficiency in the protein homeostasis in the cells or tissue of the patient.

78. The method of claim 77 wherein the condition is a loss of function disorder and wherein the method comprises identifying a deficiency in the folding or trafficking of the protein.

79. The method of claim 77 wherein the condition is a gain of function disorder and wherein the method comprises identifying a deficiency in the degradation of the protein.

80. The method of claim 79 wherein the deficiency is in the synthesis of a protein chaperone.

81. The method of claim 79 wherein the proteostasis regulator upregulates signaling via a heat shock response (HSR) pathway, an unfolded protein response (UPR) pathway, a Ca²⁺ signaling pathway, or a combination thereof.

82. The method of claim 79 wherein the proteostasis regulator upregulates transcription or translation of one or more protein chaperones, one or more folding enzymes, or a combination thereof.

83. The method of claim 79 wherein the proteostasis regulator inhibits degradation of one or more protein chaperones, one or more folding enzymes, or a combination thereof.

84. The method of claim 77 wherein the proteostasis regulator upregulates an aggregation pathway or a disaggregation pathway.

85. A method for designing a treatment regimen by identifying two or more proteostasis components which comprises comparing the activities of the proteostasis components with a standard; selecting proteostasis regulators to modify the activities of the proteostasis components towards the activities of the standard; and administering said regulators to a patient in need thereof.


SEQUENCE LISTING (46 sequences)

SEQ ID NO: 1 - DNA, 17 bp, Artificial Sequence
  Description of Artificial Sequence Synthetic primer
gtcggagtcaacggatt

SEQ ID NO: 2 - DNA, 17 bp, Artificial Sequence
  Description of Artificial Sequence Synthetic primer
aagcttcccgttctcag

SEQ ID NO: 3 - DNA, 16 bp, Artificial Sequence
  Description of Artificial Sequence Synthetic primer
cgccgaggagaagttc

SEQ ID NO: 4 - DNA, 18 bp, Artificial Sequence
  Description of Artificial Sequence Synthetic primer
catcaatgtccatgcctt

SEQ ID NO: 5 - DNA, 17 bp, Artificial Sequence
  Description of Artificial Sequence Synthetic primer
ggaggcggagaagtaca

SEQ ID NO: 6 - DNA, 18 bp, Artificial Sequence
  Description of Artificial Sequence Synthetic primer
gctgatgatggggttaca

SEQ ID NO: 7 - DNA, 19 bp, Artificial Sequence
  Description of Artificial Sequence Synthetic primer
gataaaccctgaccattcc

SEQ ID NO: 8 - DNA, 24 bp, Artificial Sequence
  Description of Artificial Sequence Synthetic primer
aagacaggagcgcagtttcataaa

SEQ ID NO: 9 - DNA, 20 bp, Artificial Sequence
  Description of Artificial Sequence Synthetic primer
aagtttcctcctccctgtcc

SEQ ID NO: 10 - DNA, 20 bp, Artificial Sequence
  Description of Artificial Sequence Synthetic primer
cgggctaaggctttacttgg

SEQ ID NO: 11 - DNA, 20 bp, Artificial Sequence
  Description of Artificial Sequence Synthetic primer
cacccagctggtttgacact

SEQ ID NO: 12 - DNA, 21 bp, Artificial Sequence
  Description of Artificial Sequence Synthetic primer
tgacagagaacctgtccttct

SEQ ID NO: 13 - DNA, 21 bp, Artificial Sequence
  Description of Artificial Sequence Synthetic primer
gcctgtatttctagacctgcc

SEQ ID NO: 14 - DNA, 20 bp, Artificial Sequence
  Description of Artificial Sequence Synthetic primer
ttcatcttgccagccagttg

SEQ ID NO: 15 - DNA, 20 bp, Artificial Sequence
  Description of Artificial Sequence Synthetic primer
ggccagtttggtgtcggttt

SEQ ID NO: 16 - DNA, 21 bp, Artificial Sequence
  Description of Artificial Sequence Synthetic primer
cgttccccgtcctagagtgtt

SEQ ID NO: 17 - DNA, 19 bp, Artificial Sequence
  Description of Artificial Sequence Synthetic primer
gcgttgtggggcagatgat

SEQ ID NO: 18 - DNA, 19 bp, Artificial Sequence
  Description of Artificial Sequence Synthetic primer
ccggttgaggtgcatcagt

SEQ ID NO: 19 - DNA, 21 bp, Artificial Sequence
  Description of Artificial Sequence Synthetic primer
aagttctacggtgacgaggag

SEQ ID NO: 20 - DNA, 22 bp, Artificial Sequence
  Description of Artificial Sequence Synthetic primer
gtcgatgttctgctcatgtttc

SEQ ID NO: 21 - DNA, 22 bp, Artificial Sequence
  Description of Artificial Sequence Synthetic primer
accaagggagaaccaggaaacg

SEQ ID NO: 22 - DNA, 22 bp, Artificial Sequence
  Description of Artificial Sequence Synthetic primer
tcaccattcggtcaatcagagc

SEQ ID NO: 23 - DNA, 21 bp, Artificial Sequence
  Description of Artificial Sequence Synthetic primer
ttacgagagaaaactcatggc

SEQ ID NO: 24 - DNA, 22 bp, Artificial Sequence
  Description of Artificial Sequence Synthetic primer
gggtccaagttgtccagaatgc

SEQ ID NO: 25 - DNA, 19 bp, Artificial Sequence
  Description of Artificial Sequence Synthetic primer
ctccatccgcacctacacc

SEQ ID NO: 26 - DNA, 22 bp, Artificial Sequence
  Description of Artificial Sequence Synthetic primer
atcaggggtatcttgagcttgg

SEQ ID NO: 27 - DNA, 18 bp, Artificial Sequence
  Description of Artificial Sequence Synthetic primer
cgtcgcctcctacctgct

SEQ ID NO: 28 - DNA, 24 bp, Artificial Sequence
  Description of Artificial Sequence Synthetic primer
ccattcagctcactgataaccttg

SEQ ID NO: 29 - DNA, 16 bp, Artificial Sequence
  Description of Artificial Sequence Synthetic primer
cgccgaggagaagttc

SEQ ID NO: 30 - DNA, 18 bp, Artificial Sequence
  Description of Artificial Sequence Synthetic primer
catcaatgtccatgcctt

SEQ ID NO: 31 - DNA, 17 bp, Artificial Sequence
  Description of Artificial Sequence Synthetic primer
ggaggcggagaagtaca

SEQ ID NO: 32 - DNA, 18 bp, Artificial Sequence
  Description of Artificial Sequence Synthetic primer
gctgatgatggggttaca

SEQ ID NO: 33 - DNA, 19 bp, Artificial Sequence
  Description of Artificial Sequence Synthetic primer
gataaaccctgaccattcc

SEQ ID NO: 34 - DNA, 24 bp, Artificial Sequence
  Description of Artificial Sequence Synthetic primer
aagacaggagcgcagtttcataaa

SEQ ID NO: 35 - DNA, 16 bp, Artificial Sequence
  Description of Artificial Sequence Synthetic primer
ccgcaaagtgaacgag

SEQ ID NO: 36 - DNA, 16 bp, Artificial Sequence
  Description of Artificial Sequence Synthetic primer
tgatggttcgtctgcc

SEQ ID NO: 37 - DNA, 18 bp, Artificial Sequence
  Description of Artificial Sequence Synthetic primer
atgaccactctcagcgtc

SEQ ID NO: 38 - DNA, 18 bp, Artificial Sequence
  Description of Artificial Sequence Synthetic primer
ctccttggctttgtcgta

SEQ ID NO: 39 - DNA, 21 bp, Artificial Sequence
  Description of Artificial Sequence Synthetic primer
gcctgtatttctagacctgcc

SEQ ID NO: 40 - DNA, 20 bp, Artificial Sequence
  Description of Artificial Sequence Synthetic primer
ttcatcttgccagccagttg

SEQ ID NO: 41 - DNA, 20 bp, Artificial Sequence
  Description of Artificial Sequence Synthetic primer
ggccagtttggtgtcggttt

SEQ ID NO: 42 - DNA, 21 bp, Artificial Sequence
  Description of Artificial Sequence Synthetic primer
cgttccccgtcctagagtgtt

SEQ ID NO: 43 - DNA, 19 bp, Artificial Sequence
  Description of Artificial Sequence Synthetic primer
gcgttgtggggcagatgat

SEQ ID NO: 44 - DNA, 19 bp, Artificial Sequence
  Description of Artificial Sequence Synthetic primer
ccggttgaggtgcatcagt

SEQ ID NO: 45 - DNA, 21 bp, Artificial Sequence
  Description of Artificial Sequence Synthetic primer
aagttctacggtgacgaggag

SEQ ID NO: 46 - DNA, 22 bp, Artificial Sequence
  Description of Artificial Sequence Synthetic primer
gtcgatgttctgctcatgtttc